(19) United States
(12) Patent Application Publication
(10) Pub. No.: US 2022/0211858 A1
(43) Pub. Date: Jul. 7, 2022

(54) TUBULAR SUPRAMOLECULAR POLYMERS

(71) Applicant: THE JOHNS HOPKINS UNIVERSITY, Baltimore, MD (US)

(72) Inventors: Honggang Cui, Lutherville, MD (US); Hao Su, Baltimore, MD (US); Feihu Wang, Baltimore, MD (US)

(21) Appl. No.: 17/604,661

(22) PCT Filed: Apr. 22, 2020
(86) PCT No.: PCT/US2020/029233

§ 371 Date: Oct. 18, 2021

Related U.S. Application Data
(60) Provisional application No. 62/836,768, filed on Apr. 22, 2019.

(51) Int. Cl. A61K 47/64; A61K 31/4745; A61K 47/60; A61K 49/00; A61P 35/00; A61K 47/69; A61K 31/337

(52) U.S. Cl. CPC A61K 47/645; A61K 31/4745; A61K 47/60; A61K 49/0095; A61K 49/0021; A61K 31/337; A61K 47/6907; A61K 47/6949; A61K 47/6903; A61K 49/0039; A61P 35/00

(57) ABSTRACT

The present invention provides the design of a class of prodrugs for self-assembly into therapeutic tubular supramolecular polymers and their use in a wide variety of applications. The therapeutic tubular supramolecular polymers can be used to formulate drugs and imaging agents for in vitro and in vivo uses.

### REFERENCE TO RELATED APPLICATIONS

This application claims the benefit of U.S. Provisional Patent Application No. 62/836,768, filed on Apr. 22, 2019, which is hereby incorporated by reference for all purposes as if fully set forth herein.

### STATEMENT OF GOVERNMENTAL INTEREST

This invention was made with government support under grant no. R21CA191740 awarded by the National Institutes of Health, and grant no. DMR 1255281 awarded by the National Science Foundation. The government has certain rights in the invention.

### BACKGROUND OF THE INVENTION

The self-assembly of monomeric units into supramolecular polymers (SPs) emulates the key features of biological systems (1, 2), enabling the creation of new electrical (3, 4), optical (5, 6), biological and/or pharmaceutical functionalities (7, 8) that the individual monomers do not possess. For example, peptide amphiphiles can become highly bioactive after their assembly into supramolecular nanofibers, with emerging properties for specific cell signaling attributed to the high-density display of epitopes that exists only in their supramolecular form (9). Another example is the cooperative association of hexabenzocoronene conjugates into graphitic nanotubes with significant electronic properties arising from intermolecular π-π stacking (10). In other cases, assembly into larger objects can suppress the biological or pharmaceutical activities of the individual building units, leading to a complete loss of potency (11). This feature can be utilized to develop effective drug delivery systems as the functionality of the monomeric units can be restored through a spatiotemporally controlled disassembly process (12-14). In this regard, molecular assembly serves as a means to switch on and off the system or individual functionalities.

### SUMMARY OF THE INVENTION

In accordance with some embodiments, the present invention provides the design of a class of self-assembling prodrugs (SAPDs) of various CMCs that all self-assemble into SPs. Some of the SAPDs of the present invention are camptothecin (CPT) analogues, termed Tubustecans (TTs), which upon dissolution in aqueous solutions assemble into tubular supramolecular polymers that mask the pharmaceutical nature of the unassembled CPT. Upon dissociation in biologically relevant environments, the CPT activity can be effectively restored.

CPT is a natural product originally isolated from the bark and stem of the Chinese Happy Tree, with two analogues currently used in the clinic (15).

In accordance with some embodiments, the present invention provides a composition comprising one or more hydrophilic drug molecules covalently linked to at least one or more biodegradable carbonate linkers which are covalently linked to one or more hydrophilic peptides, and may comprise an additional small molecule of interest.

In accordance with an embodiment, the present invention provides a Tubustecan compound having the following formula:

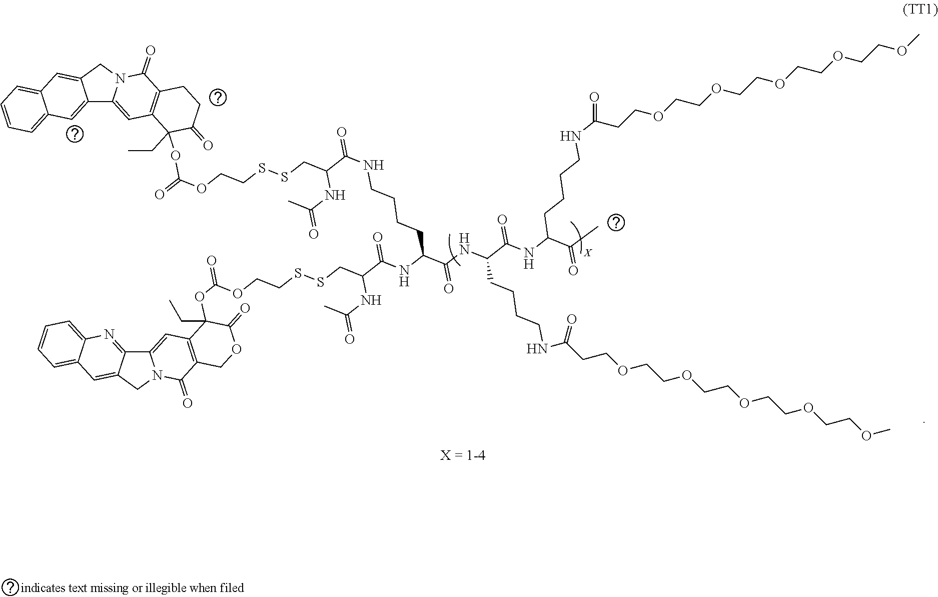

In accordance with another embodiment, the present invention provides a composition comprising a Tubustecan compound having the following formula:

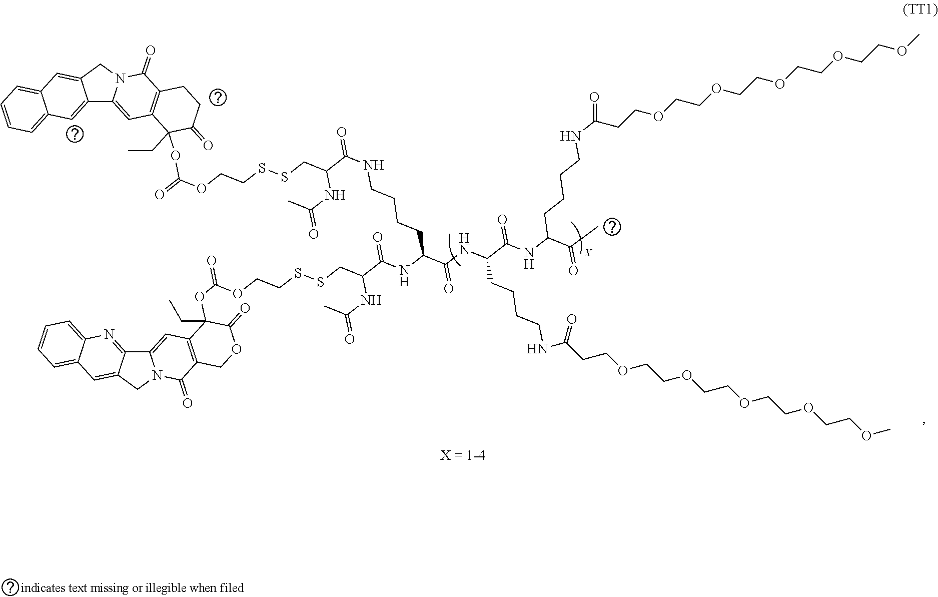

and a pharmaceutically acceptable carrier.

In some embodiments, two or more Lys molecules conjugated with oligoethylene glycol groups are added to the di(Cys) portion of the molecule as needed.

In accordance with an embodiment, the present invention provides a Tubustecan compound having the following formula:

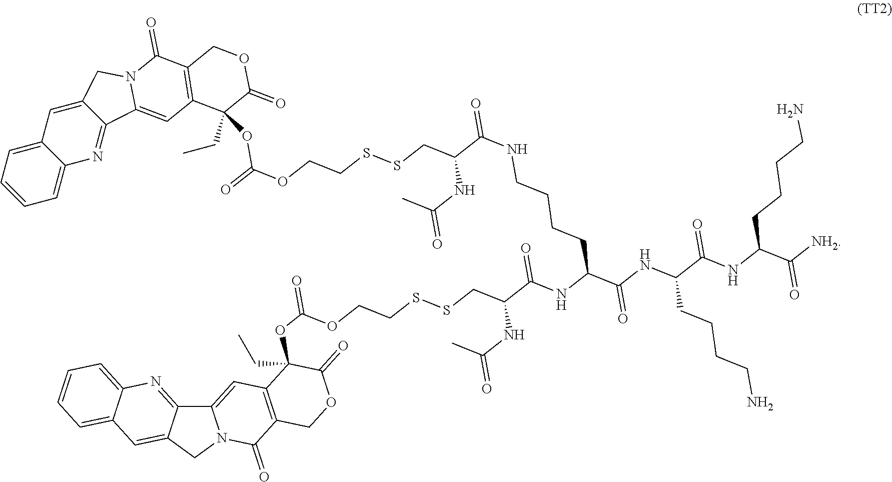 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCCCN)C(=O)N[C@@H](CCCCN)C(N)=O)C(=O)OCc2c1cc1n(c2=O)Cc2cc3ccccc3nc2-1]

In accordance with another embodiment, the present invention provides a composition comprising a Tubustecan compound having the following formula:

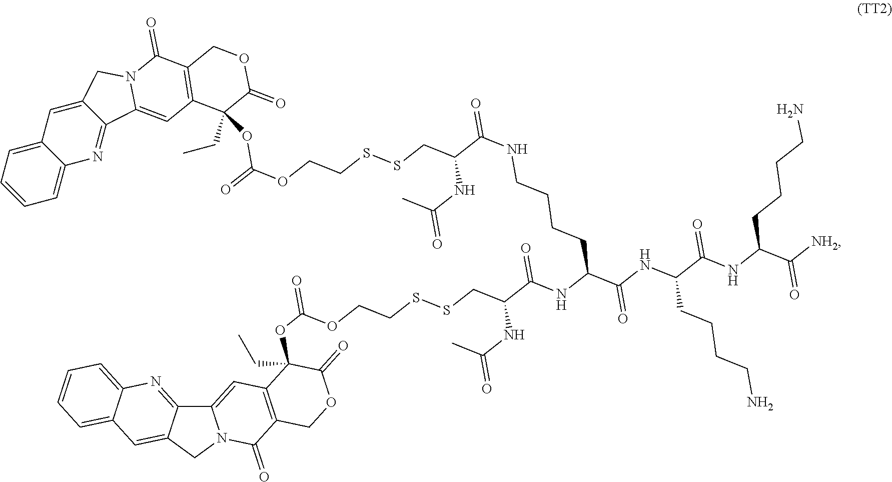 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCCCN)C(=O)N[C@@H](CCCCN)C(N)=O)C(=O)OCc2c1cc1n(c2=O)Cc2cc3ccccc3nc2-1]

and a pharmaceutically acceptable carrier.

In accordance with an embodiment, the present invention provides a Tubustecan compound having the following formula:

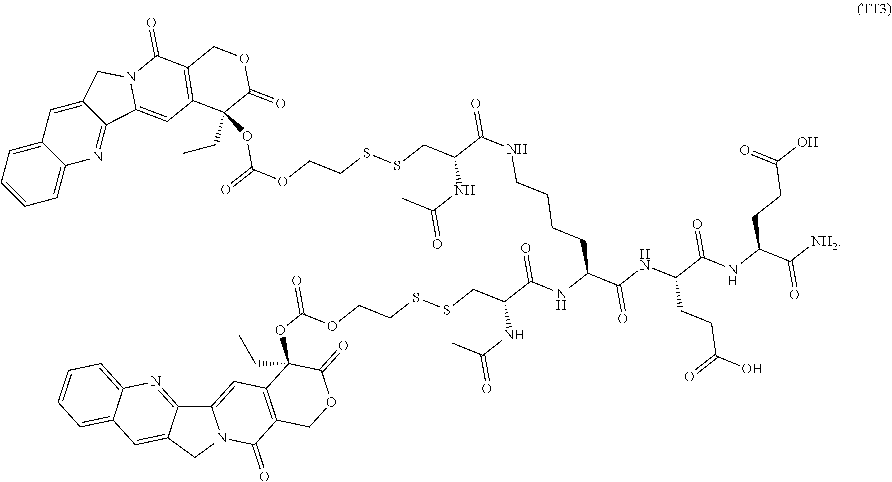 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCC(=O)O)C(=O)N[C@@H](CCC(=O)O)C(N)=O)C(=O)OCc2c1cc1n(c2=O)Cc2cc3ccccc3nc2-1]

In accordance with another embodiment, the present invention provides a composition comprising a Tubustecan compound having the following formula:

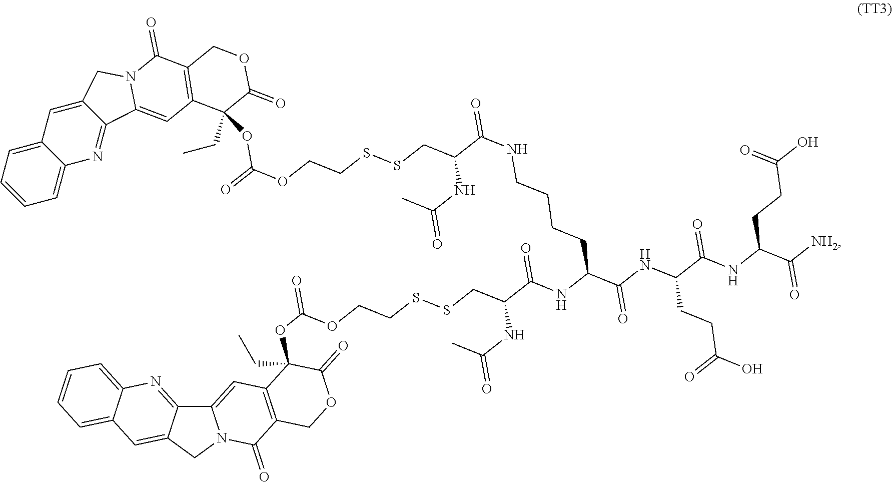 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCC(=O)O)C(=O)N[C@@H](CCC(=O)O)C(N)=O)C(=O)OCc2c1cc1n(c2=O)Cc2cc3ccccc3nc2-1]

and a pharmaceutically acceptable carrier.

In accordance with an embodiment, the present invention provides a Tubustecan compound having the following formula:

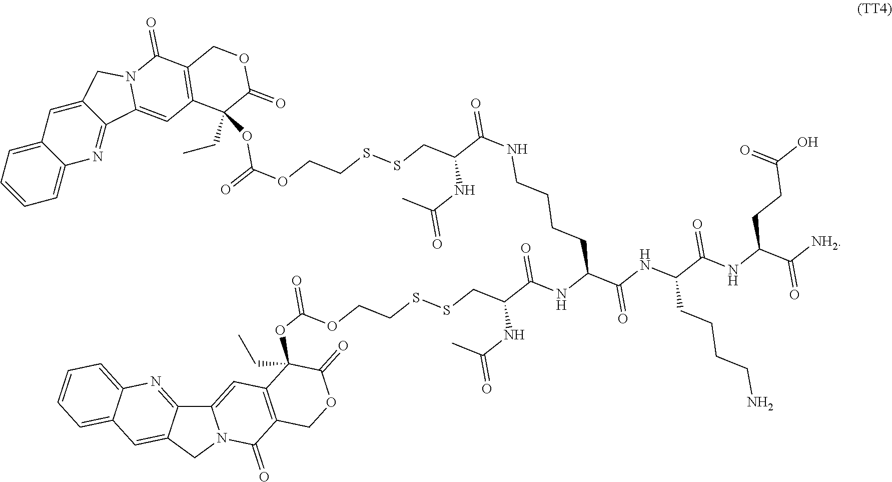 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCCCN)C(=O)N[C@@H](CCC(=O)O)C(N)=O)C(=O)OCc2c1cc1n(c2=O)Cc2cc3ccccc3nc2-1]

In accordance with another embodiment, the present invention provides a composition comprising a Tubustecan compound having the following formula:

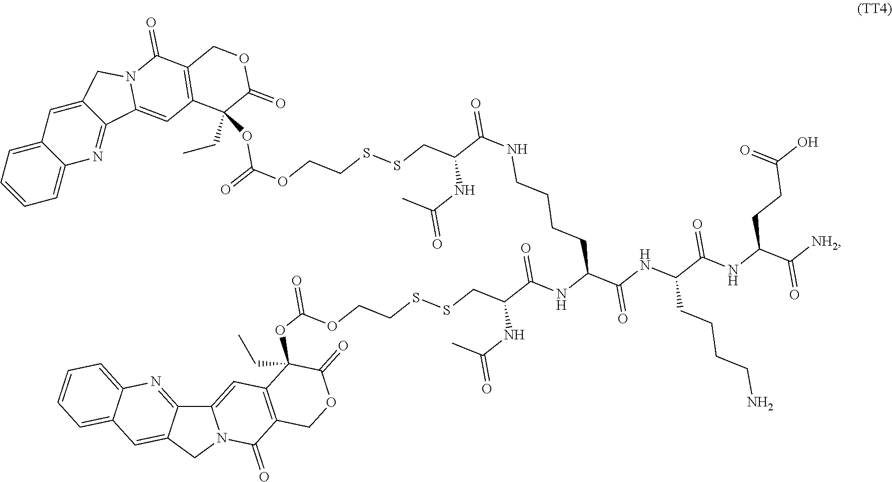 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCCCN)C(=O)N[C@@H](CCC(=O)O)C(N)=O)C(=O)OCc2c1cc1n(c2=O)Cc2cc3ccccc3nc2-1]

and a pharmaceutically acceptable carrier.

In accordance with an embodiment, the present invention provides a Tubustecan compound having the following formula:

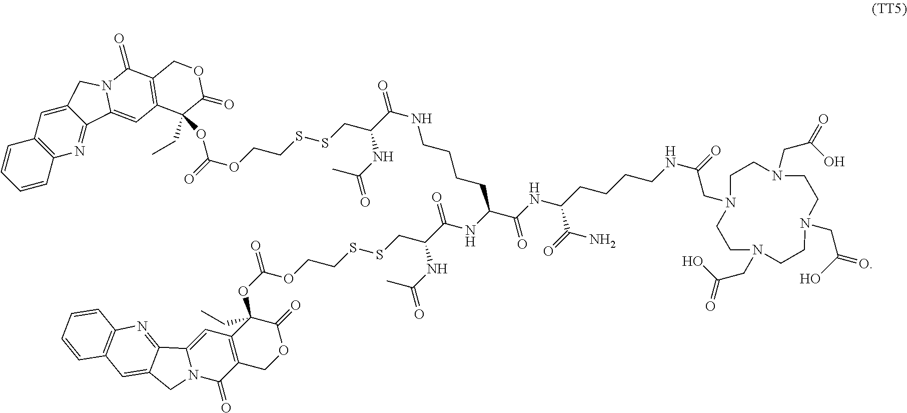 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@H](CCCCNC(=O)CN2CCN(CC(=O)O)CCN(CC(=O)O)CCN(CC(=O)O)CC2)C(N)=O)C(=O)OCc2c1cc1n(c2=O)Cc2cc3ccccc3nc2-1]

In accordance with another embodiment, the present invention provides a composition comprising a Tubustecan compound having the following formula:

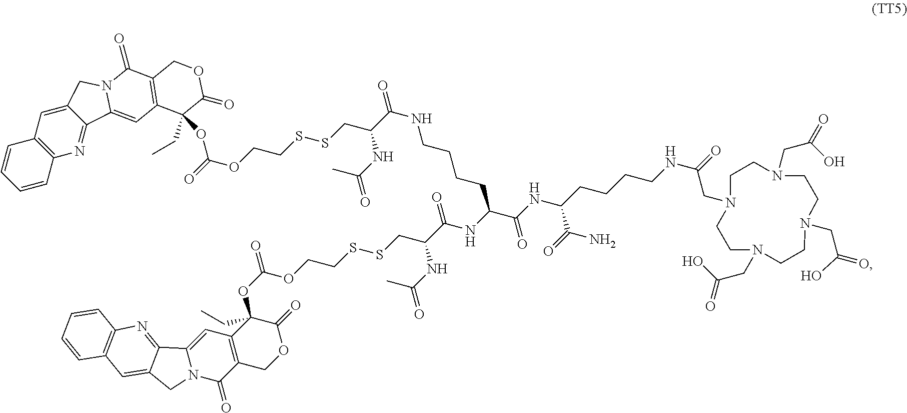 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@H](CCCCNC(=O)CN2CCN(CC(=O)O)CCN(CC(=O)O)CCN(CC(=O)O)CC2)C(N)=O)C(=O)OCc2c1cc1n(c2=O)Cc2cc3ccccc3nc2-1]

and a pharmaceutically acceptable carrier.

In accordance with one or more embodiments, the present invention provides methods for administration of one or more biologically active agents to a cell or population of cells comprising administering to the subject an effective amount of at least one or more compounds described above.

In accordance with one or more embodiments, the present invention provides methods for administration of one or more biologically active agents to a cell or population of cells comprising administering to the subject an effective amount of at least one or more compositions described above.

In accordance with one or more embodiments, the present invention provides methods for administration of one or more biologically active agents to a cell or population of cells comprising administering to the subject an effective amount of at least one or more compositions described above, and at least one additional biologically active agent.

In accordance with one or more embodiments, the present invention provides methods for administration of one or more biologically active agents to a subject in need thereof, comprising administering to the subject an effective amount of at least one or more compounds described above.

In accordance with one or more embodiments, the present invention provides methods for administration of one or more biologically active agents to a subject in need thereof, comprising administering to the subject an effective amount of at least one or more compositions described above.

In accordance with one or more embodiments, the present invention provides methods for administration of one or more biologically active agents to a subject in need thereof, comprising administering to the subject an effective amount of at least one or more compositions described above, and at least one additional biologically active agent.

In accordance with one or more embodiments, the present invention provides methods for making the compounds and compositions described above.

### BRIEF DESCRIPTION OF THE DRAWINGS

FIGS. 1A-1E. Molecular design and tubular assembly of non-ionic Tubustecan 1 (TT 1). (1A) Chemical structure of TT 1. Representative cryo-TEM (1B) and conventional low- (1C) and high-magnification (1D) TEM micrographs of supramolecular nanotubes formed by self-assembly of TT 1 in water. The dark central line shown in (1D) observed in all filamentous assemblies suggests their tubular nature. The inset image of a toroid in (1D) further confirms the tubular structures (scale bar: 5 nm). Solution concentrations: 800 μM for cryo-TEM imaging; 200 μM for conventional TEM imaging. (1E) Representative circular dichroism (CD) spectrum of the assembled TT 1 nanotubes in water at a concentration of 200 μM.

FIGS. 2A-2Q. The chemical design and molecular assembly of ionic Tubustecans (TT 2-TT 5). (2A) Chemical structures of cationic TT 2 (2A), anionic TT 3 (2B), zwitterionic TT 4 (2C), and DOTA-containing TT 5 (2D). Cryo-TEM (2E, 2F, 2G, 2H) and conventional low- (2I, 2J, 2K, 2L) and high-magnification (2M, 2H, 2O, 2P) TEM micrographs reveal the tubular assembly for all the designed TT molecules: TT 2 (2E, 2I, 2M), TT 3 (2F, 2J, 2N), TT 4 (2G, 2K, 2Q), and TT 5 (2H, 2L, 2P). White arrows in (2M) and (2Q) point to the occasionally observed toroidal structures that further supports the tubular nature of the observed filamentous assemblies. Concentrations: 800 μM for cryo-TEM imaging; 200 μM for conventional TEM imaging. Inserted images in (2E), (2F), (2G) demonstrate self-supporting hydrogels formed by TT 2, TT 3, and TT 4, respectively, in PBS buffer at 5 mM.

FIGS. 3A-3L. In vitro and in vivo evaluation of Tubustecan drug release and efficacy as systemic and local therapies. (3A) Representative RP-HPLC trace of the free CPT release from TT 1 tubular SPs at different time points (concentration: 200 μM). (3B) Drug release profile of TT 2 from its self-assembling hydrogels at 10 mM in a DPBS buffer. The TT 2 conjugate was released linearly, with ˜10% of TT 2 released over 31 days. The inset photographs show that the TT 2 gel remained at the bottom of the vial after 1-month release. (3C) In vitro toxicity of TT 1 and TT 2 against the U87 MG brain cancer cell line, with both free CPT and Irinotecan as controls (48 h incubation). (3D) Maximum tolerated dose (MTD) study of TT 1. A single dose of TT 1 was administrated through i.v. injection, and body weights of athymic nude mice were recorded for 15 days (n=3). Doses of 54 and 36 mg/kg are not summarized because they caused at least 1 death in each group. (3E) Antitumor efficacy study of varying doses of TT 1 (4.5 mg/kg, 9 mg/kg, and 15 mg/kg of CPT equivalent), with non-treatment, free CPT (i.p. injection) and irinotecan (i.p. injection) as controls (n=5). (3F) Cumulative survival plot of mice via systemic delivery. Loss of mice was a result of treatment-related death or euthanasia after the predetermined end point was reached. (3G) Plasma concentration of TT 1 at 4.5 mg/kg and 15 mg/kg with free CPT (4.5 mg/kg) as a control (i.v. injection). Total CPT concentration (3H) and free CPT concentration 3(I) in tumor site with time. (3J) Representative photographs of PBS control (left) and TT 2 hydrogels (right) injected subcutaneously in athymic nude mice. (3K) Antitumor efficacy, and (3L) survival plot of TT 2 (10 mM, 30 μL) via local delivery (control group n=5 and TT 2 group n=7). All data are presented as mean±s.d. and analyzed by one-way ANOVA (Fisher; 0.01<*P<0.05; **P<0.01; ***P<0.001).

FIGS. 4A-4G. TT1 tubular supramolecular polymers as a universal dispersing agent for small-molecule hydrophobes. (4A) A set of optical images showing the effective encapsulation of various hydrophobic dye molecules into TT 1 aqueous solution (left to right: Control (without any dye), Coumarin 6, Neil Red, Rose Bengal lactone, and IR 780). (4B) Absorption and emission profiles (excitation at 740 nm) of IR 780-containing TT 1 solution. Emission spectrum is measured with 15% acetonitrile. TEM micrographs of TT 1 nanotubes after loading with Coumarin 6 (4C), Neil Red (4D), Rose Bengal lactone (4E) and IR 780 (4F) suggest that the tubular features remain intact, albeit with slightly enlarged diameters of 9.8±1.4 nm (4C), 9.6±1.2 nm (4F), 10.7±1.4 nm (4G), and 10.2±0.9 nm (4G).

FIGS. 5A-5C. Schematic illustration for synthesis of Tubustecans (TTs). (5A) Synthetic routes to peptide segments dCys-K₂ and dCys-OEG₂ using standard Fmoc solid phase peptide techniques (dCys-E₂ and dCys-KE use similar protocols to dCys-K₂). (5B) Synthesis of functional TT 1-4 by mixing peptide segments synthesized in (A) with CPT-etcSS-Pyr in DMSO. (5C) Synthesis of DOTA-containing TT 5 by coupling dCPT-K with DOTA.

FIGS. 6A-6E. RP-HPLC chromatograms and ESI-MS spectra of TT 1 (A), TT 2 (B), TT 3 (C), TT 4 (D) and TT 5 (E). These data suggested the successful synthesis and purification of all five TT molecules studied in the report.

FIG. 7. TEM image of self-assembled TT 1 that illustrates the occasional presence of toroidal structures. The wall thickness and diameter of inner cavities of TT 1 nanotubes were determined by measuring these observed toroids (n>50).

FIGS. 8A-8B. CD spectra of different TT nanotubes (A) and normalized spectra (B) in aqueous solution at pH=7.4. After normalizing by maximum intensity, the CD spectra for all five nanotubes showed similar pattern, further confirming the semblable tubular morphology of five nanostructures. The concentrations are 200 μM.

FIG. 9. Critical micellization concentrations (CMC) of the TTs were measured by encapsulation of Nile Red. CMC values of the nanotubes are within the range of 2-5 μM regardless of the hydrophilic segment, confirming again the dominant role of the CPT units in stabilizing their supramolecular assemblies. Note: panel F provides a summary of the transition curves of each TT for ease of comparison. The mechanism of Nile Red encapsulation method is that the Nile Red dye fluoresces intensely in hydrophobic environments (encapsulated) and is strongly quenched and red-shifted in aqueous media (unencapsulated). Plotting the ratio of intensity at 635 nm (emission maximum of the encapsulated dye) to that at 660 nm (emission maximum in aqueous conditions) against the concentration of TTs shows the transition that occurs when the concentration of TT monomers exceeds the CMC.

FIGS. 10A-10D. ζ-Potential values of self-assembled TT 1-5 in 1×-DPBS buffer at pH 7.4 (A) and their variation over time (days 1, 2, 4 and 7) (B); ζ-Potential values of self-assembled TT 1-5 in 1×-DPBS buffer at pH 5.0 (C) and their variation over time (D). The average values and their standard deviations are calculated from three measurements. As expected, TT 2 tubules carrying two lysine residues show a positive value of 22.1 mV at pH 7.4 and 31.8 mV at pH 5.0 due to the increased protonation of lysine amines. Both TT 3 and TT5 tubules show more negative ζ-potentials at pH 7.4 than 5.0 due to the incorporation of multiple carboxylic groups. The zwitterionic TT 4 tubules carry a more negative charge at pH 7.4 (−18.8 mV) than pH 5.0 (−2.3 mV), likely due to the placement of glutamic acid at the C-terminus. In contrast, the non-ionic TT 1 nanotubes are resistant to the changes in solution pH, having a consistent negative value of −5 mV at both pH 7.4 and 5.0. In addition, the ζ-potential values of TTs are stable over seven days, indicating their long-term stability.

FIG. 11. Drug release plot of TT 1 at a concentration of 200 μM with or without 10 mM GSH in buffer. About 80% of the conjugated CPT molecules were released within 2 h in the presence of 10 mM GSH, reaching almost 100% by 6 h, while only a slight amount (less than 10%) of the conjugates had degraded (by hydrolysis) in 24 h without GSH. These results indicate that the therapeutic supramolecular polymers are able to undergo bioconversion to the parent drug and exert the pharmaceutical and biological functionalities of monomeric CPTs.

FIG. 12. Stability of TT 1 upon dilution in cell medium (phenol red free) containing 10% fetal bovine serum. No significant changes were observed from the normalized curves at concentrations above 25 μM, indicating no obvious disassociation of the self-assembled nanotubular structures. Further dilution of TT 1 solution to 10 and 5 μM resulted in a slight decrease of CD signal at 389 nm, suggesting that higher monomer/nanostructure ratio and likely partial dissociation of nanostructures as the concentration approaches the CMC. In addition, the inset photograph shows that a solution of TT 1 in cell medium remains clear after a week, suggesting no obvious aggregation of nanotubes in cell medium and long-term stability. All solutions were prepared and incubated for two days before CD measurements were taken.

FIG. 13. Maximum tolerated dose (MTD) study showing the averaged lowest body weight recorded per corresponding dosage of TT 1 (n=3 for each dosage group). MTD was determined by intravenously administering TT 1 in a dose escalation study in healthy athymic nude mice. The MTD of TT 1 was in the range of 24-30 mg/kg (CPT equivalent), which greatly exceeds MTD of free CPT (˜5 mg/kg). The maximum tolerated dose (MTD) was defined by the largest dose that did not result in more than a 20% mean body weight loss or death of an animal in that group. Doses of 54 and 36 mg/kg caused at least one death in each group.

FIGS. 14A-14B. Body weight change of mice (A) and cumulative survival plot of mice (B) in systemic delivery of TT 1. Groups treated with drugs (except the 4.5 mg/kg group) showed slight body weight decreases (less than 10%), however, they were all within the acceptable toxicity range. Much improved median survivals of mice were observed for TT 1 at 9 mg/kg (37 d) and 15 mg/kg (43 d) respectively, compared with control (11 d), free CPT (17 d), TT 1 at 4.5 mg/kg (23 d), and irinotecan (27 d).

FIGS. 15A-15B. Representative photographs of a PBS bolus (A) and TT 2 hydrogel (B) injected subcutaneously in athymic nude mice. While the PBS bolus control (indicated by a red arrow in A) disappeared in less than five minutes, the TT 2 formed a yellowish hydrogel (indicated by red arrow in B) immediately after injection and remained in place for weeks.

FIGS. 16A-16D. TT 2 hydrogel for local cancer treatment. (A) Representative photographs of a complete tumor disappearance in mice after treatment with a TT 2 hydrogel. (B) Body weight change of mice during the local delivery of a TT 2 hydrogel. (C) Antitumor efficacy and (D) survival plot of TT 2 (10 mM, 30 μL) via local delivery (control group n=5 and TT 2 group n=7). All the mice treated with TT 2 hydrogel showed significant tumor regression and survived for more than 45 days. Four out of seven mice exhibited complete tumor disappearance and survived to the end point (around 140 d), suggesting that the TT 2 hydrogel enables molecular delivery in a controlled manner and improves the therapeutic index of monomeric CPT.

FIGS. 17A-17C. TT1 nanotube as a carrier for the anticancer drug paclitaxel (PTX). (A) HPLC analysis of PTX-doped TT 1 nanotubes monitored at 220 nm. A new peak around 13 minutes corresponding to that of PTX is present and indicates successful encapsulation. The concentrations of both nanotube and PTX were determined by comparing the area under the curve of each component with its standard calibration curve, yielding an encapsulation efficiency around 11%. (B) CD spectra of PTX-doped nanotubes. A slight decrease in intensity compared with TT 1 is caused by encapsulation of bulky PTX. (C) TEM image of PTX-doped nanotubes. TEM imaging revealed the expected tubular morphology of PTX-doped nanotubes with a diameter of 10.6±0.9 nm, which is around 1 nm wider than pure TT 1 nanotubes. All concentrations are 200 μM and scale bar for TEM is 100 nm.

FIG. 18. Chemical structures of the four different dyes encapsulated within TT 1.

FIGS. 19A-19C. Schematic illustration of the design and self-assembly of self-assembling prodrugs (SAPDs). (A) Chemical structure of the designed SAPDs. (B) Cartoon of SAPD platform. Two hydrophobic camptothecin (CPT) molecules (yellow) were conjugated with four different oligoethylene-glycol (OEG)-decorated hydrophilic auxiliaries (blue) through the biodegradable etcSS linker (black) to create SAPD 1-4, respectively. (C) Illustration of self-assembly of SAPD into supramolecular polymer (SP).

FIGS. 20A-20D. Supramolecular polymers formed by SAPDs in water. Representative cryo-TEM of supramolecular assemblies of SAPD 1 (A), SAPD 2 (B), SAPD 3 (C) and SAPD 4 (D). TEM images reveal that all the prodrugs self-assembled into one-dimensional structures. All concentrations: 2 mM.

FIGS. 21A-21H. CMC, stability and drug release studies of SAPDs. (A) CMC measurement of SAPDs using a Nile Red method. CMCs of SAPD 1 and 2 are estimated to be 2.7 μM and 10.1 μM, respectively. CMCs of SAPD 3 and 4 exceed 200 μM, and the exact values cannot be directly determined here. (B) CD spectra of SAPDs at 200 μM in water. SAPD 1 shows very strong absorptions attributed to CPT chromophore interactions and intermolecular hydrogen bonding. SAPD 2 shows a similar pattern with largely reduced intensities. The lack of typical hydrogen bonding interactions and characteristic CPT absorptions in SAPD 3 and 4 indicates that they may not form 1D nanostructures at the concentration of 200 μM. The chromophore absorptions can be ascribed to intramolecular CPT interactions within a single prodrug. (C) CD spectra of SAPDs at 200 μM in 10% rat plasma. No apparent difference in the absorptions of SAPD 1 and 2 were observed compared with those in water, while slight changes can be seen in the cases of SAPD 3 and 4. (D) Plots of absorption of SAPD 1 and SAPD 2 assemblies at 389 nm in the time- and concentration-dependent CD measurement. Drug release plots of SAPDs at 200 μM in PBS (E) and rat plasma (G) with 10 mM GSH. Cumulative drug degradation plots of SAPDs at 200 μM in PBS (F) and rat plasma (H) without GSH.

FIGS. 22A-22B In vitro cell cytotoxicity of SACPDs against HT-29 colorectal adenocarcinoma cells (A) and HCT-116 colorectal carcinoma cells (B), with both free CPT and irinotecan as controls (72 h incubation).

FIGS. 23A-23F. In vivo antitumor efficacy and circulation study of SAPDs at the same dose (10 mg/kg, CPT equivalent). SAPDs were i.v. injected q4dx4 (black arrows) at days 1, 5, 9 and 13 at a dose of 10 mg/kg mice (n=6). Blank PBS group and free CPT (i.p.) at a dose of 9 mg/kg (q4dx4) at days 1, 5, 9, and 13 were taken as controls (n=5), however administration of CPT resulted in death of all five mice after the second dose. Irinotecan (i.p.) at a dose of 100 mg/kg (qwkx3, white arrows) at days 1, 8 and 15 was another control (n=5). Tumor volume (A), body weight (B) and cumulative survival (C) plots of mice. Loss of mice is a result of treatment-related death or euthanasia after predetermined end point was reached. All the data are presented as mean±s.d. and analyzed by one-way ANOVA (Fisher; 0.01<*P<0.05; **P<0.01; ***P<0.001). Real-time concentration of total CPT (D) and bounded CPT in SAPDs (E) in the circulation study on SD rats (n=3) at 5 min, 15 min, 30 min, 1 h, 2 h, 4 h, 8 h, and 12 h. (F) The ratio of bounded CPT in SAPDs to total CPT within 1 h after injection.

FIGS. 24A-24C. In vivo antitumor efficacy of SAPDs at their respective estimated MTDs. SAPDs were i.v. injected q4dx3 (black arrows) on days 1, 5 and 9 at doses of 12 mg/kg (CPT equivalent) for SAPD 1, and 36 mg/kg for all other three SAPDs (n=5). Blank PBS group (n=5) was taken as a control and irinotecan (i.p.) at a dose of 100 mg/kg (qwkx3, red arrows) on days 1, 8 and 15 was another control (n=5). Tumor volume (A), body weight (B) and cumulative survival (C) plots of mice. Slight decrease of body weight was observed in all treated groups with one treatement related death in the SAPD 2 group. All the data are presented as mean±s.d. and analyzed by one-way ANOVA (Fisher; 0.01<*P<0.05; **P<0.01; ***P<0.001).

FIG. 25 depicts Scheme 1, an illustration of the circulation fate of an supramolecular polymer (SP) after entering into the circulation. SP (1) may dissociate into fragments/monomers in the plasma upon dilution, and the dissociation kinetics is mostly dictated by its CMC. SP has the tendency to accumulate more in the tumor (2) and major organs (3), liver, spleen, kidney, lung, heart), while fragments/monomers (4) tend to undergo a rapid renal clearance. Thus, the lower the CMC of a SP, the lower the percentage of fragments and monomers, leading to reduced excretion and enhanced tumor (improved efficacy) and healthy organ uptake (increased toxicity).

### DETAILED DESCRIPTION OF THE INVENTION

The present invention provides the design of a class of camptothecin analogs for self-assembly into therapeutic tubular supramolecular polymers and their use in a wide variety of applications. The emergence of system functionalities in monomer activity suppression, transportation, accumulation, and retention accounts for their superior performance in animal studies to free CPT and irinotecan. At the same time, the dynamic and reversible nature of non-covalent interactions involved enables their effective conversion into functional monomeric units through the requisite dissociation and subsequent degradation. The robustness of the tubular assembly protocol allows for incorporating a variety of surface groups for biological interfacing and other functional units to expand their inherent functionality. Essentially, these self-assembling CPT analogues are also self-formulating, and self-delivering, making them the simplest drug delivery system studied so far.

In accordance with some embodiments, the present invention provides a composition comprising one or more hydrophilic drug molecules covalently linked to at least one or more biodegradable carbonate linkers which are covalently linked to one or more hydrophilic peptides.

As used herein, the term “hydrophobic drug molecules” roughly describes a heterogeneous group of molecules that exhibit poor solubility in water but that are typically, but certainly not always, soluble in various organic solvents. Often, the terms slightly soluble (1-10 mg/ml), very slightly soluble (0.1-1 mg/ml), and practically insoluble (<0.1 mg/ml) are used to categorize such substances. Drugs such as steroids and many anticancer drugs are important classes of poorly water-soluble drugs; however, their water solubility varies over at least two orders of magnitudes. Typically, such molecules require secondary solubilizers such as carrier molecules, liposomes, polymers, or macrocyclic molecules such as cyclodextrins to help the hydrophobic drug molecules dissolve in aqueous solutions necessary for drug delivery in vivo. Other types of hydrophobic drugs show even a lower aqueous solubility of only a few ng/ml. Since insufficient solubility commonly accompanies undesired pharmacokinetic properties, the high-throughput screening of kinetic and thermodynamic solubility as well as the prediction of solubility is of major importance in discovery (lead identification and optimization) and development.

In some embodiments, the hydrophobic drug molecules can include camptothecin, irinotecan and other analogs, such as 7-[2-(N-isopropylamine)ethyl]-(20S)-CPT (belotecan) and active metabolites, such as 7-ethyl-10-hydroxy-CPT.

In some embodiments, the hydrophobic drug molecules can include taxol and derivatives such as paclitaxel, docetaxel, 10-deacetylbaccatin III, baccatin III, paclitaxel C, and 7-epipaclitaxel, for example.

In some embodiments, the hydrophobic drug molecules can include other hydrophobic drug molecules, for example, doxorubicin, curcumin, ciprofloxacin and others.

In some embodiments, the biodegradable carbonate linkers include disulfanylbutyrate (buSS), disulfanylethylcarbonate (etcSS), for example.

The drug-linker can be conjugated to the hydrophilic peptide via established protein conjugation methodologies including, but not limited to, disulfide formation via reaction of cysteine-thiol with an activated thiol, thioether formation via reaction of cysteine-thiol with a maleimide, triazole formation via copper-assisted azide-alkyne cycloaddition (CuAAC) and other “Click” reactions.

As used herein, the term “biodegradable linkers” refers to a small molecule or peptide fragment that is capable of covalently linking the hydrophobic drug molecule to the hydrophilic peptide in the present invention. These covalent linkages must be sufficiently labile to be hydrolyzed or cleaved when in the target cell or organ of a subject. In certain embodiments, the linker bonds are preferably cleaved off in the target organ or cell by an enzyme or cellular component that is at a higher concentration in the target microenvironment than in the body or outside of the target cell or organ. Examples of such linker moieties include, but are not limited to amides, disulfides, polyamino acids, biopolymers, esters, aldehydes, hydrazones and the like.

In accordance with an embodiment, the biodegradable linkers of the present invention include (4-(pyridin-2-yldisulfanyl)butanoate) (buSS). The buSS linker has a disulfide moiety that allows it to be reductively cleaved primarily intracellularly by glutathione. In particular, the concentration of glutathione inside tumor cells is 100 to 1000 times higher than in the interstitial fluid, thus allowing the compositions of the present invention to act as a prodrug and enter the cell intact. Once inside the cell, the reduction of the linker bonds by glutathione occurs, and the free hydrophobic drug molecule can act on its target. It will be understood by those of ordinary skill in the art that other linker moieties can be used where they interact with the hydrophilic peptide in a similar manner.

The hydrophilic peptides increase the aqueous solubility of the drug and can promote the formation of well-defined nanostructure architectures including, but not limited to, cylindrical or spherical micelles, and hollow nanotubes.

In accordance with some embodiments, the one or more hydrophilic peptides can be neutral, cationic, anionic, zwitterionic, and can comprise chelating agents.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma.-carboxyglutamate, and O-phosphoserine.

The term “amino acid analogs,” refers to compounds that have the same basic chemical structure as a naturally occurring amino acid, e.g., an alpha. carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid “mimetics” refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of ordinary skill in the art recognizes that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typical conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Amino acids which are cationic include Arginine (R), Histidine (H), and Lysine (K). Amino acids which are anionic include Aspartic acid (D) and Glutamic acid (E).

In some embodiments, the hydrophilic peptides are covalently linked to a hydrophilic polymer. Polymer is used to refer to molecules composed of repeating monomer units, including homopolymers, block copolymers, heteropolymers, random copolymers, graft copolymers and so on. “Polymers” also include linear polymers as well as branched polymers, with branched polymers including highly branched, dendritic, and star polymers.

A monomer is the basic repeating unit in a polymer. A monomer may itself be a monomer or may be dimer or oligomer of at least two different monomers, and each dimer or oligomer is repeated in a polymer.

Biocompatible polymer, biocompatible cross-linked polymer matrix and biocompatibility are art-recognized. For example, biocompatible polymers include polymers that are neither themselves toxic to the host (e.g., and animal or human), nor degrade (if the polymer degrades) at a rate that produces monomeric or oligomeric subunits or other byproducts at toxic concentrations in the host.

“Biodegradable” is art-recognized, and includes monomers, polymers, polymer matrices, gels, compositions and formulations, such as those described herein, that are intended to degrade during use, such as in vivo. Biodegradable polymers and matrices typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain, functional group and so on to the polymer backbone. In certain embodiments, one or the other or both general types of biodegradation may occur during use of a polymer. As used herein, the term “biodegradation” encompasses both general types of biodegradation.

Suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), and poly(ethylene oxide)-co-poly(propylene oxide) block copolymers (poloxamers and meroxapols), for example.

In accordance with an embodiment, the present invention provides a self-assembling prodrug Tubustecan molecule comprising the following formula:

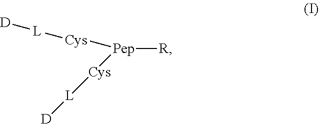

wherein D is a hydrophobic drug molecule, L is a hydrolysable linker, Cys is cysteine, Pep is a hydrophilic peptide of at least two amino acids with a free side chain, and R is H, or a hydrophilic molecule of choice. In some embodiments, Pep is Lys-Lys, and/or Lys-Glu, and/or Glu-Glu, for example.

It will be understood by those of ordinary skill in the art, that in some embodiments, D can represent two or more different hydrophobic drug molecules. For example, D can include a first drug (D1) and second drug (D2) which can be, for example, chemotherapeutic agents which are not the same.

Without being limited to any particular example, the pharmaceutical composition of the present invention can be a hetero-dual drug amphiphile comprising a first drug molecule of camptothecin (CPT) and a second drug molecule of paclitaxel (PXL) linked by the same or different linker, for example buSS, to the PEP portion, for example.

In accordance with an alternative embodiment, the drug amphiphiles of the present invention can be linked with an additional peptide, or other small molecule (R).

In accordance with an embodiment, the present invention provides a Tubustecan compound having the following formula:

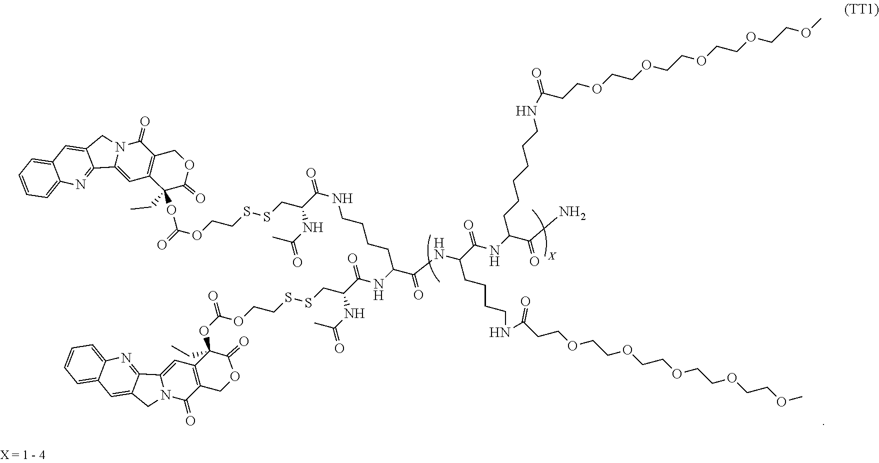

In accordance with another embodiment, the present invention provides a composition comprising a Tubustecan compound having the following formula:

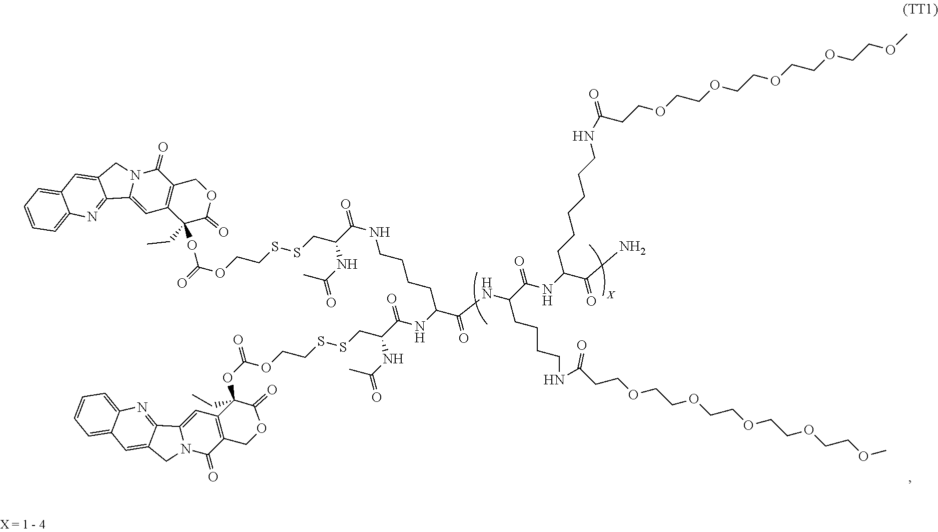

and a pharmaceutically acceptable carrier.

In the above aspect, the compound has a biologically compatible polymer as a hydrophilic component. It will be understood that in this example, the Pep moiety is a di-Lys moiety covalently linked to an oligoethylene glycol (OEG) molecule of varying length. In some embodiments, the OEG is 2-10 ethylene glycol molecules in length. In some other embodiments, it can be a di-Glu moiety, or a Lys-Glu moiety. Any combination of hydrophilic amino acids with a free side chain of 2-10 amino acids can be used.

For example, in some aspects the molecules of formula TT1 can include:

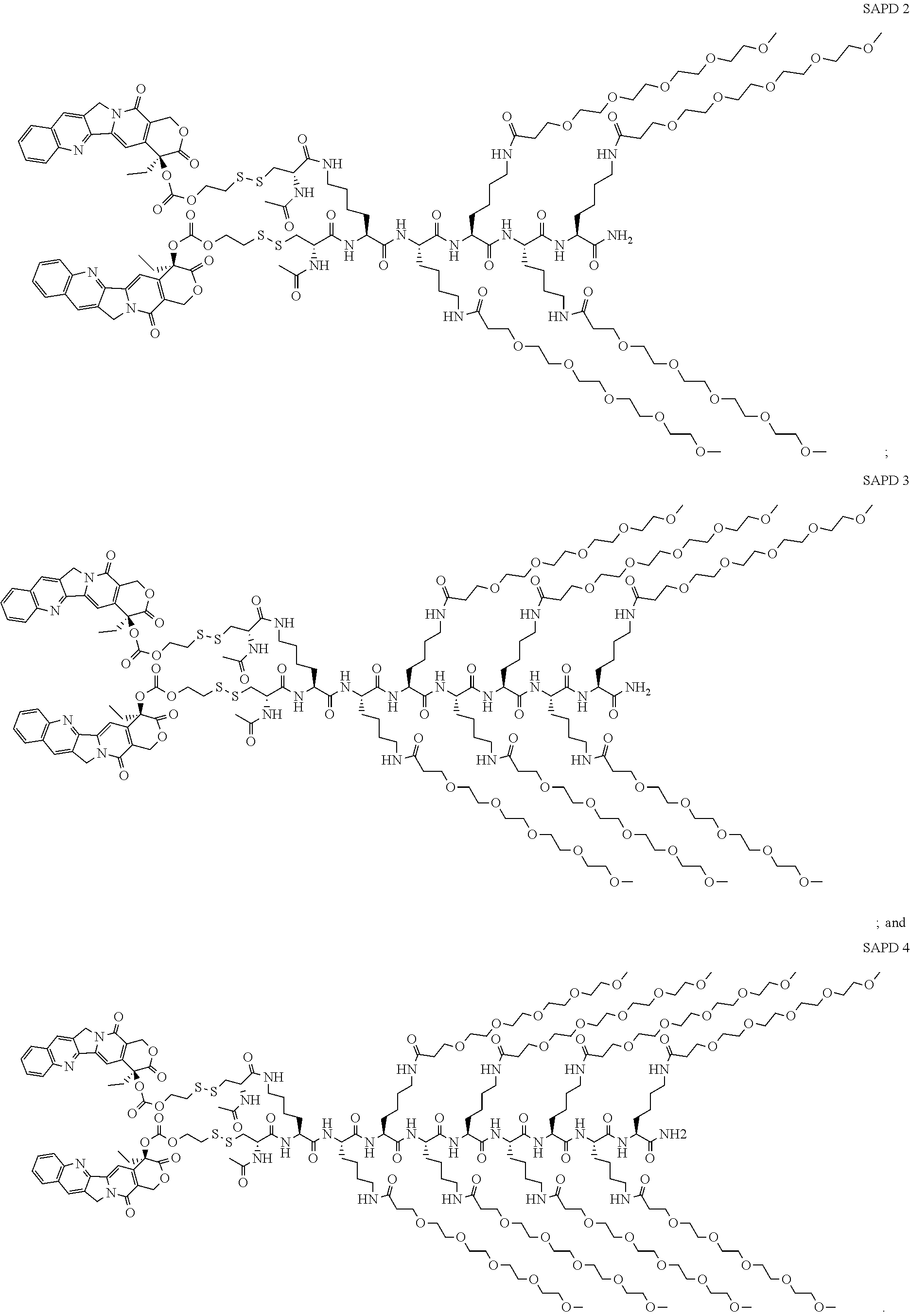

In accordance with an embodiment, the present invention provides a Tubustecan compound having the following formula:

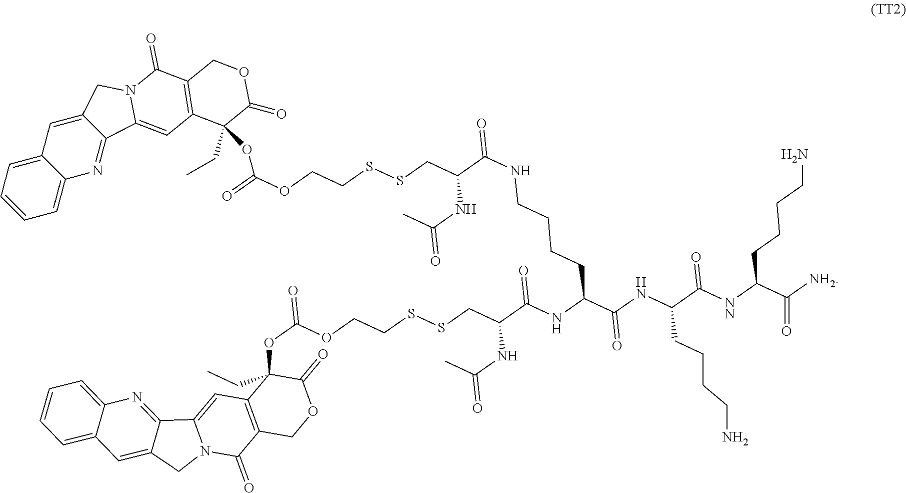

In accordance with another embodiment, the present invention provides a composition comprising a Tubustecan compound having the following formula:

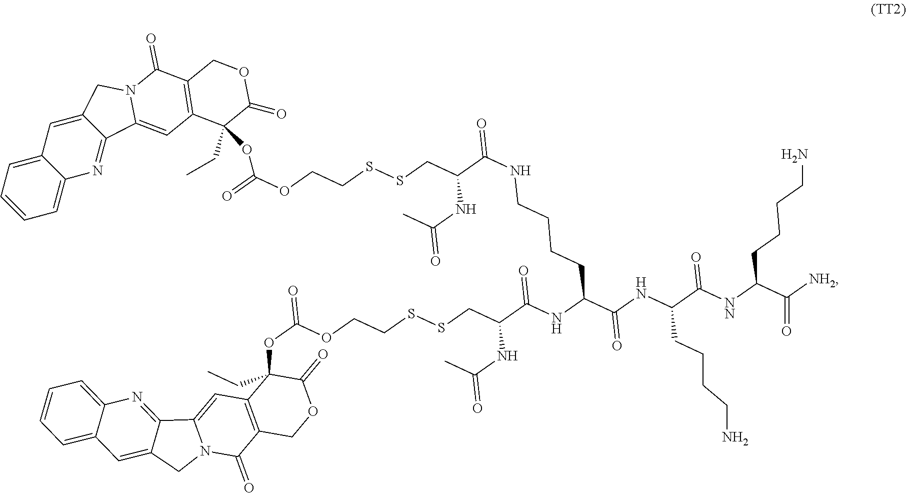

and a pharmaceutically acceptable carrier.

In the above aspect, the compound has one or more cationic amino acids as a hydrophilic component.

In accordance with an embodiment, the present invention provides a Tubustecan compound having the following formula:

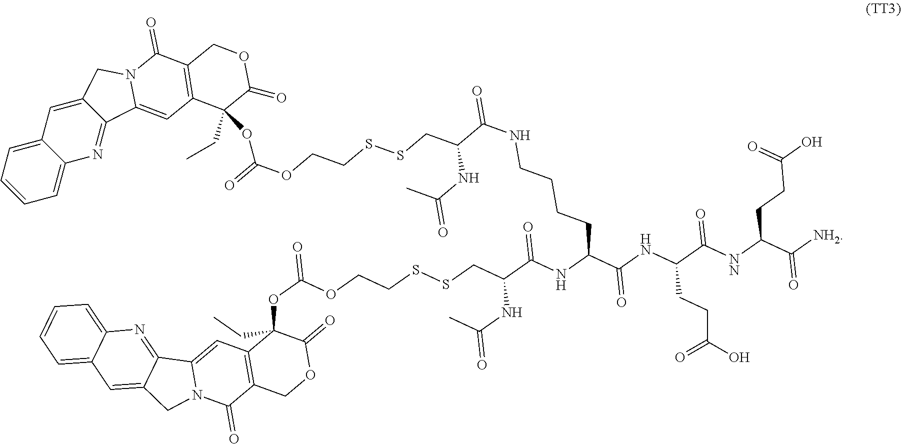

In accordance with another embodiment, the present invention provides a composition comprising a Tubustecan compound having the following formula:

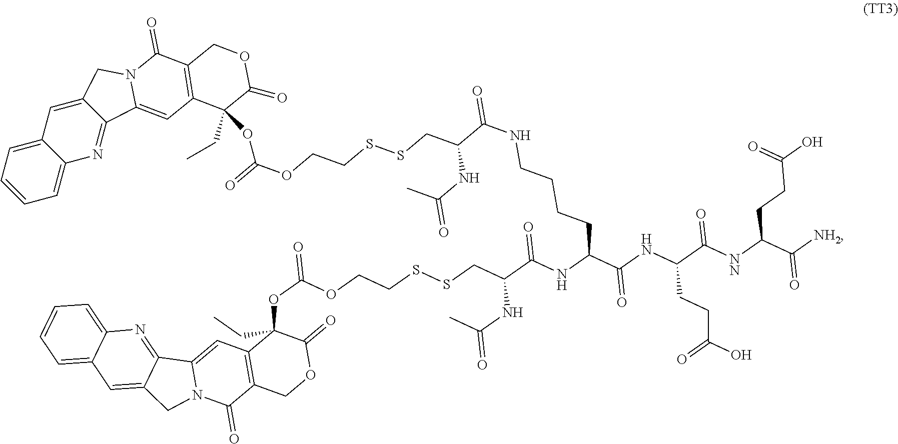

and a pharmaceutically acceptable carrier.

In the above aspect, the compound has one or more anionic amino acids as a hydrophilic component.

In accordance with an embodiment, the present invention provides a Tubustecan compound having the following formula:

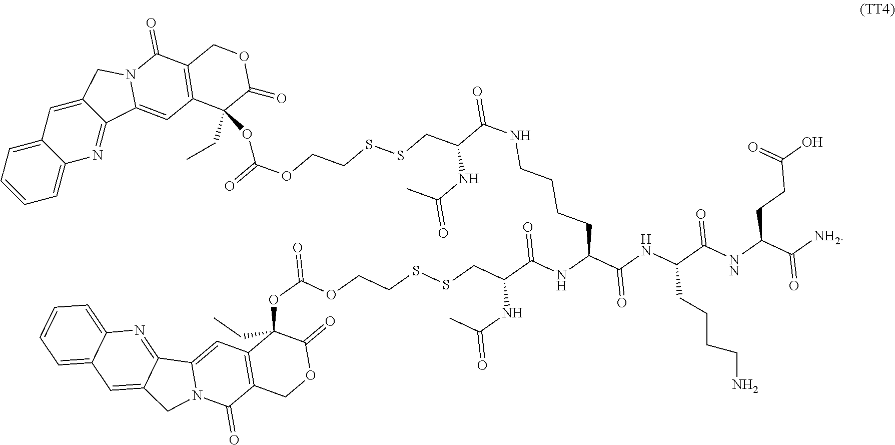

In accordance with another embodiment, the present invention provides a composition comprising a Tubustecan compound having the following formula:

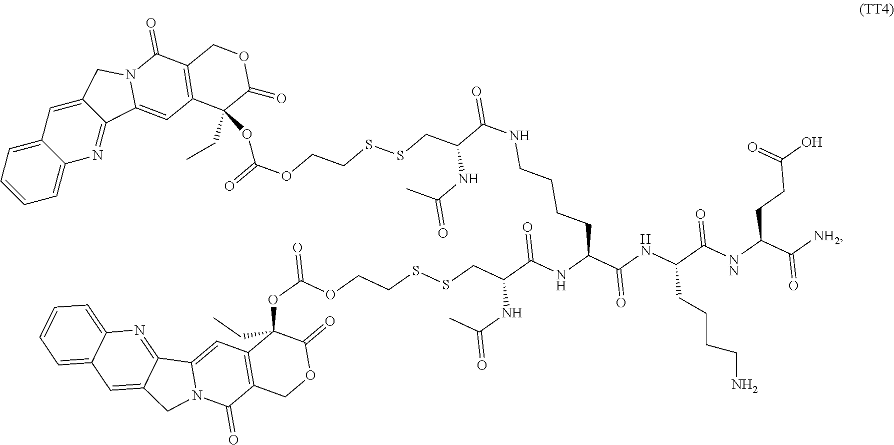

and a pharmaceutically acceptable carrier.

In the above aspect, the compound has one or more cationic and anionic (zwitterionic) amino acids as a hydrophilic component.

In accordance with an embodiment, the present invention provides a Tubustecan compound having the following formula:

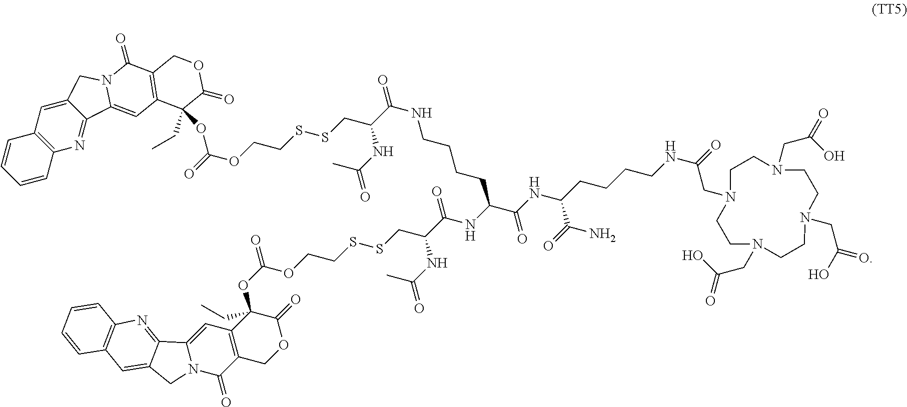 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@H](CCCCNC(=O)CN2CCN(CC(=O)O)CCN(CC(=O)O)CCN(CC(=O)O)CC2)C(N)=O)C(=O)OCc2c1cc1n(c2=O)Cc2cc3ccccc3nc2-1]

In accordance with another embodiment, the present invention provides a composition comprising a Tubustecan compound having the following formula:

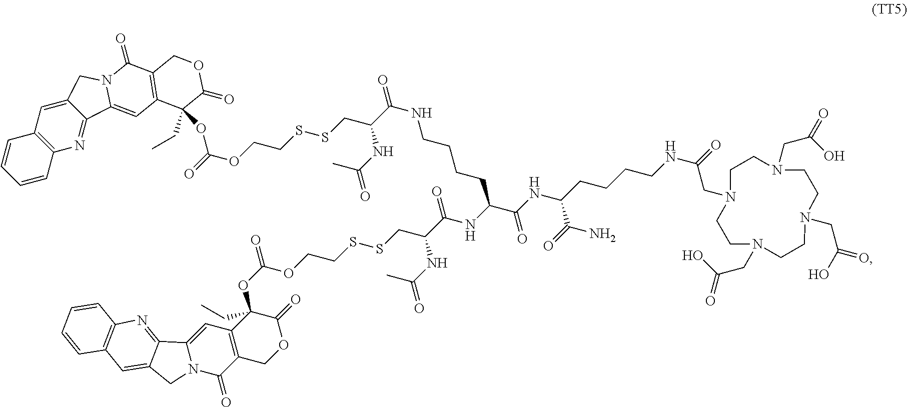 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@H](CCCCNC(=O)CN2CCN(CC(=O)O)CCN(CC(=O)O)CCN(CC(=O)O)CC2)C(N)=O)C(=O)OCc2c1cc1n(c2=O)Cc2cc3ccccc3nc2-1]

and a pharmaceutically acceptable carrier.

In the above aspect, the compound has one or more chelating moieties as a hydrophilic component.

It will be understood that each of TT2-TT5 molecules can have 2 or more OEG moieties covalently attached at the free side chains, just as exemplified in TT1 above.

The molecular design of the four SAPDs studied (in TT1), comprising two hydrophobic CPT molecules and OEG-decorated peptides of various OEG numbers (2, 4, 6 and 8). The design rationale herein is that the CPT moiety, in addition to its pharmacological role, can provide directional, associative π-π interactions to contribute to the self-assembly process. By fixing the number of CPTs and varying the hydrophilicity of peptide segments, we are able to create four SAPDs of different hydrophilic-lipophilic balances (HLB), leading to formation of supramolecular polymers of different stability. The use of the OEG segment to modify the side chain of lysine endows a non-ionic and neutral surface chemistry to the resultant SPs, so as to reduce protein absorption and increase circulation half-lives. In addition, the CPT and the hydrophilic moiety are connected via a biodegradable linker (for example, a disulfanyl-ethyl carbonate linker (etcSS)), which was shown to effectively release the parent CPT upon contact with intracellular glutathione (GSH)

As used herein the term “pharmaceutically active compound” or “therapeutically active compound” means a compound useful for the treatment or modulation of a disease or condition in a subject suffering therefrom. Examples of pharmaceutically active compounds can include any drugs known in the art for treatment of disease indications. A particular example of a pharmaceutically active compound is a chemotherapeutic agent.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66: 1-19 (1977).

The term “chemotherapeutic agent” as well as words stemming therefrom, as used herein, generally includes pharmaceutically or therapeutically active compounds that work by interfering with DNA synthesis or function in cancer cells. Based on their chemical action at a cellular level, chemotherapeutic agents can be classified as cell-cycle specific agents (effective during certain phases of cell cycle) and cell-cycle nonspecific agents (effective during all phases of cell cycle). Without being limited to any particular example, examples of chemotherapeutic agents can include alkylating agents, angiogenesis inhibitors, aromatase inhibitors, antimetabolites, anthracyclines, antitumor antibiotics, monoclonal antibodies, platinums, topoisomerase inhibitors, and plant alkaloids. Further examples of chemotherapeutic agents include asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

It will be understood that any hydrophobic chemotherapeutic agents can be conjugated to the biodegradable linker as defined in the present invention. Examples include camptothecin, paclitaxel, anthracyclines, carboplatin, cisplatin, daunorubicin, doxorubicin, methotrexate, vinblastine, vincristine, etc.

For purposes of the invention, the amount or dose of the compositions of the present invention that is administered should be sufficient to effectively target the cell, or population of cells in vivo, such that cell apoptosis or death in the target cell or population of cells occurs in the subject over a reasonable time frame. The dose will be determined by the efficacy of the particular pharmaceutical formulation and the location of the target population of cells in the subject, as well as the body weight of the subject to be treated.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent, “pharmacologically active agent” and “drug” are used, then, it is to be understood that the invention includes the active agent per se, as well as pharmaceutically acceptable, pharmacologically active salts, esters, amides, prodrugs, metabolites, analogs etc.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released the subject composition, for example, into adjacent tissues or fluids upon administration to a subject. In some embodiments, a biologically active agent may be used in cross-linked polymer matrix of this invention, to, for example, promote cartilage formation. In other embodiments, a biologically active agent may be used in cross-linked polymer matrix of this invention, to treat, ameliorate, inhibit, or prevent a disease or symptom, in conjunction with, for example, promoting cartilage formation.

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and genes that can be incorporated into the compositions of the invention. Non-limiting examples of biologically active agents include following: adrenergic blocking agents, anabolic agents, androgenic steroids, antacids, anti-asthmatic agents, anti-allergenic materials, anti-cholesterolemic and anti-lipid agents, anti-cholinergics and sympathomimetics, anti-coagulants, anti-convulsants, anti-diarrheal, anti-emetics, anti-hypertensive agents, anti-infective agents, anti-inflammatory agents such as steroids, non-steroidal anti-inflammatory agents, anti-malarials, anti-manic agents, anti-nauseants, anti-neoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, benzophenanthridine alkaloids, biologicals, cardioactive agents, cerebral dilators, coronary dilators, decongestants, diuretics, diagnostic agents, erythropoietic agents, estrogens, expectorants, gastrointestinal sedatives, agents, hyperglycemic agents, hypnotics, hypoglycemic agents, ion exchange resins, laxatives, mineral supplements, mitotics, mucolytic agents, growth factors, neuromuscular drugs, nutritional substances, peripheral vasodilators, progestational agents, prostaglandins, psychic energizers, psychotropics, sedatives, stimulants, thyroid and antithyroid agents, tranquilizers, uterine relaxants, vitamins, antigenic materials, and prodrugs.

Specific examples of useful biologically active agents the above categories include: anti-neoplastics such as androgen inhibitors, antimetabolites, cytotoxic agents, and immunomodulators; anti-tussives such as dextromethorphan, hydrobromide, noscapine, carbetapentane citrate, and chlophedianol hydrochloride; antihistamines such as chlorpheniramine phenindamine tartrate, pyrilamine doxylamine succinate, and phenyltoloxamine citrate; decongestants such as hydrochloride, phenylpropanolamine hydrochloride, pseudoephedrine hydrochloride, and ephedrine; various alkaloids such as codeine phosphate, codeine sulfate, and morphine; mineral supplements such as potassium chloride, zinc chloride, calcium carbonate, magnesium oxide, and other alkali metal and alkaline earth metal salts; ion exchange resins such as such as N-acetylprocainamide; antipyretics and analgesics such as acetaminophen, aspirin and ibuprofen; appetite suppressants such as phenyl-propanol amine or caffeine; expectorants such as guaifenesin; antacids such as aluminum hydroxide and magnesium hydroxide; biologicals such as peptides, polypeptides, proteins and amino acids, hormones, interferons or cytokines and other bioactive peptidic compounds, such as calcitonin, ANF, EPO and insulin; anti-infective agents such as antifungals, antivirals, antiseptics and antibiotics; and desensitizing agents and antigenic materials, such as those useful for vaccine applications.

More specifically, non-limiting examples of useful biologically active agents include the following therapeutic categories: analgesics, such as nonsteroidal anti-inflammatory drugs, opiate agonists and salicylates; antihistamines, such as H1-blockers and H2-blockers; anti-infective agents, such as antihelmintics, antianaerobics, antibiotics, aminoglycoside antibiotics, antifungal antibiotics, cephalosporin antibiotics, macrolide antibiotics, miscellaneous antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, and urinary antiinfectives; antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics; autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, and adrenergic agonist sympathomimetics; cardiovascular agents, such as antianginals, antianginals, calcium-channel blocker antianginals, nitrate antianginals, antiarrhythmics, cardiac glycoside antiarrhythmics, class I antiarrhythmics, class antiarrhythmics, class antiarrhythmics, class IV antiarrhythmics, antihypertensive agents, a-blocker antihypertensives, angiotensin-converting enzyme inhibitor (ACE inhibitor) antihypertensives, β-blocker antihypertensives, calcium-channel blocker antihypertensives, central-acting adrenergic antihypertensives, diuretic antihypertensive agents, peripheral vasodilator antihypertensives, antilipemics, bile acid sequestrant antilipemics, reductase inhibitor antilipemics, inotropes, cardiac glycoside inotropes, and thrombolytic agents; dermatological agents, such as antihistamines, anti-inflammatory agents, corticosteroid anti-inflammatory agents, anesthetics, topical antiinfectives, topical antiinfectives, antiviral topical antiinfectives, and topical antineoplastics; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hematological agents, such as antianemia agents, hematopoietic antianemia agents, coagulation agents, anticoagulants, hemostatic coagulation agents, platelet inhibitor coagulation agents, thrombolytic enzyme coagulation agents, and plasma volume expanders; hormones and hormone modifiers, such as abortifacients, adrenal agents, corticosteroid adrenal agents, androgens, anti-androgens, antidiabetic agents, sulfonylurea antidiabetic agents, antihypoglycemic agents, oral contraceptives, progestin contraceptives, estrogens, fertility agents, oxytocics, parathyroid agents, pituitary hormones, progestins, antithyroid agents, thyroid hormones, and tocolytics; immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal antiinflammatory drugs, salicylate anti-inflammatory agents, skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists; ophthalmic agents, such as anti-glaucoma agents, anti-glaucoma agents, mitotics, anti-glaucoma agents, mydriatics, adrenergic agonist mydriatics, antimuscarinic mydriatics, ophthalmic anesthetics, ophthalmic anti-infectives, ophthalmic aminoglycoside anti-infectives, ophthalmic macrolide anti-infectives, ophthalmic quinolone anti-infectives, ophthalmic sulfonamide anti-infectives, ophthalmic tetracycline anti-infectives, ophthalmic anti-inflammatory agents, ophthalmic corticosteroid antiinflammatory agents, and ophthalmic nonsteroidal anti-inflammatory drugs; psychotropic agents, such as antidepressants, heterocyclic antidepressants, monoamine oxidase inhibitors selective serotonin re-uptake inhibitors tricyclic antidepressants, antimanics, antipsychotics, phenothiazine antipsychotics, anxiolytics, sedatives, and hypnotics, barbiturate sedatives and hypnotics, benzodiazepine anxiolytics, sedatives, and hypnotics, and psychostimulants; respiratory agents, such as antitussives, bronchodilators, adrenergic agonist bronchodilators, antimuscarinic bronchodilators, expectorants, mucolytic agents, respiratory antiinflammatory agents, and respiratory corticosteroid antiinflammatory agents; toxicology agents, such as antidotes, heavy agents, substance abuse agents, deterrent substance abuse agents, and withdrawal substance abuse agents; minerals; and vitamins, such as vitamin A, vitamin B, vitamin C, vitamin D, vitamin E, and vitamin K.

Other classes of biologically active agents from the above categories include: analgesics in general, such as lidocaine, other “caine” analgesics or derivatives thereof, and nonsteroidal anti-intlammatory drugs (NSAIDs) analgesics, including diclofenac, ibuprofen, ketoprofen, and naproxen; opiate agonist analgesics, such as codeine, fentanyl, hydromorphone, and morphine; salicylate analgesics, such as aspirin (ASA) (enteric coated ASA); H1-blocker antihistamines, such as clemastine and terfenadine; H2-blocker antihistamines, such as cimetidine, famotidine, nizadine, and ranitidine; anti-infective agents, such as mupirocin; antianaerobic antiinfectives, such as chloramphenicol and clindamycin; antifungal antibiotic antiinfectives, such as amphotericin b, clotrimazole, fluconazole, and ketoconazole; macrolide antibiotic antiinfectives, such as azithromycin and erythromycin; miscellaneous antibiotic antiinfectives, such as and imipenem; penicillin, antibiotic anti-infectives, such as nafcillin, oxacillin, penicillin G, and penicillin V; quinolone antibiotic anti-infectives, such as ciprofloxacin and nortfloxacin; tetracycline antibiotic antiinfectives, such as doxycycline, minocycline and tetracycline; antituberculosis antimycobacterial antiinfectives such as isoniazid and rifampin; antiprotozoal antiinfectives, such as atovaquone and dapsone; antimalarial antiprotozoal antiinfectives, such as chloroquine and pyrimethamine; anti-retroviral antiinfectives, such as ritonavir and zidovudine; antiviral anti-infective agents, such as acyclovir, ganciclovir, interferon-γ, and rimantadine; alkylating antineoplastic agents, such as carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; vinca alkaloid natural antineoplastics, such as vinblastine and vincristine; autonomic agents, such as nicotine; anticholinergic autonomic agents, such as benztropine and trihexyphenidyl; antimuscarinic anticholinergic autonomic agents, such as atropine and oxybutynin; ergot alkaloid autonomic agents, such as bromocriptine; cholinergic agonist parasympathomimetics, such as pilocarpine; cholinesterase inhibitor parasympathomimetics, such as pyridostigmine; α-blocker sympatholytics, such as prazosin; β-blocker sympatholytics, such as atenolol; adrenergic sympathomimetics, such as albuterol and dobutamine; cardiovascular agents, such as aspirin (ASA) (enteric coated ASA); β-blocker antianginals, such as atenolol and propranolol; calcium-channel blocker antianginals, such as nifedipine and verapamil; nitrate antianginals, such as isosorbide dinitrate (ISDN); cardiac glycoside antiarrhythmics, such as class I antiarrhythmics, such as lidocaine, mexiletine, phenytoin, procainamide, and quinidine; class antiarrhythmics II, such as atenolol, metoprolol, propranolol, and timolol; class III antiarrhythmics, such as amiodarone; class IV antiarrhythmics, such as diltiazem and verapamil; antihypertensives, such as prazosin; angiotensin-converting enzyme inhibitor (ACE inhibitor) antihypertensives, such as captopril and enalapril; antihypertensives, such as atenolol, metoprolol, nadolol, and propanolol; calcium-channel blocker antihypertensive agents, such as diltiazem and nifedipine; central-acting adrenergic antihypertensives, such as clonidine and methyldopa; diuretic antihypertensive agents, such as amiloride, furosemide, hydrochlorothiazide (HCTZ), and spironolactone; peripheral vasodilator antihypertensives, such as minoxidil; antilipemics, such as gemfibrozil and probucol; bile acid sequestrant antilipemics, such as cholestyramine; reductase inhibitor antilipemics, such as lovastatin and pravastatin; inotropes, such as amrinone, dobutamine, and dopamine; cardiac glycoside inotropes, such as thrombolytic agents, such as alteplase, anistreplase, streptokinase, and urokinase; dermatological agents, such as colchicine, isotretinoin, methotrexate, minoxidil, tretinoin dermatological corticosteroid anti-inflammatory agents, such as betamethasone and dexamethasone; antifungal topical antiinfectives, such as amphotericin clotrimazole, miconazole, and nystatin; antiviral topical antiinfectives, such as acyclovir; topical antineoplastics, such as electrolytic and renal agents, such as lactulose; loop diuretics, such as furosemide; potassium-sparing diuretics, such as triamterene; thiazide diuretics, such as hydrochlorothiazide (HCTZ); uricosuric agents, such as probenecid; enzymes and thrombolytic enzymes, such as alteplase, anistreplase, streptokinase and urokinase; antiemetics, such as prochlorperazine; salicylate gastrointestinal anti-inflammatory agents, such as sulfasalazine; gastric acid-pump inhibitor anti-ulcer agents, such as omeprazole;) H2-blocker anti-ulcer agents, such as cimetidine, famotidine, nizatidine, ranitidine; digestants, such as pancrelipase; prokinetic agents, such as erythromycin; opiate agonist intravenous anesthetics such as fentanyl; hematopoietic antianemia agents, such as (G-CSF), and (GM-CSF); coagulation agents, such as factors 1-10 (AHF 1-10); anticoagulants, such as warfarin; thrombolytic enzyme coagulation agents, such as alteplase, anistreplase, streptokinase and urokinase; hormones and hormone modifiers, such as bromocriptine; abortifacients, such as methotrexate; antidiabetic agents, such as insulin; oral contraceptives, such as estrogen and progestin; progestin contraceptives, such as levonorgestrel and norgestrel; estrogens such as conjugated estrogens, diethylstilbestrol (DES), estrogen (estradiol, estrone, and estropipate); fertility agents, such as clomiphene, human chorionic gonadotropin (HCG), and menotropins; parathyroid agents such as calcitonin; pituitary hormones, such as desmopressin, goserelin, oxytocin, and vasopressin (ADH); progestins, such as medroxyprogesterone, norethindrone, and progesterone; thyroid hormones, such as levothyroxine; immunobiologic agents, such as interferon beta-lb and interferon gamma-lb; immunoglobulins, such as immune globulin IgM, IgG, IgA; amide local anesthetics, as lidocaine; ester local anesthetics, such as benzocaine and procaine; musculoskeletal corticosteroid antiinflammatory agents, such as beclomethasone, betamethasone, cortisone, dexamethasone, hydrocortisone, and prednisone; musculoskeletal anti-inflammatory immunosuppressives, such as azathioprine, cyclophosphamide, and methotrexate; musculoskeletal nonsteroidal anti-inflammatory drugs such as diclofenac, ibuprofen, ketoprofen, ketorlac, and naproxen; skeletal muscle relaxants, such as and diazepam; reverse neuromuscular blocker skeletal muscle relaxants, such as pyridostigmine; neurological agents, such as nimodipine, riluzole, tacrine and ticlopidine; anticonvulsants, such as carbamazepine, gabapentin, lamotrigine, phenytoin, and valproic acid; barbiturate anticonvulsants, such as phenobarbital and primidone; benzodiazepine anticonvulsants, such as clonazepam, diazepam, and lorazepam; anti-Parkinson's' agents, such as bromocriptine, levodopa, carbidopa, and pergolide; anti-vertigo agents, such as meclizine; opiate agonists, such as codeine, fentanyl, hydromorphone, methadone, and morphine; opiate antagonists, such as naloxone; antiglaucoma agents, such as timolol; mitotic anti-glaucoma agents, such as pilocarpine; ophthalmic aminoglycoside antiinfectives, such as gentamicin, neomycin, and tobramycin; ophthalmic quinolone antiinfectives, such as ciprofloxacin, norfloxacin, and ofloxacin; ophthalmic corticosteroid anti-agents, such as dexamethasone and prednisolone; ophthalmic nonsteroidal anti-inflammatory drugs such as diclofenac; antipsychotics, such as clozapine, haloperidol, and risperidone; benzodiazepine anxiolytics, sedatives and hypnotics, such as clonazepam, diazepam, lorazepam, oxazepam, and prazepam; psychostimulants, such as methylphenidate and pemoline; such as codeine; bronchodilators, such as adrenergic agonist bronchodilators, such as albuterol; respiratory corticosteroid antiinflammatory agents, such as dexamethasone; antidotes, such as flumazenil and naloxone; heavy metal agents, such as penicillamine; deterrent substance abuse agents, such as disulfiram, naltrexone, and nicotine; withdrawal substance abuse agents, such as bromocriptine; minerals, such as iron, calcium, and magnesium; vitamin B compounds, such as cyanocobalamin (vitamin B12) and niacin (vitamin B3); vitamin C compounds, such as ascorbic acid; and vitamin D such as calcitriol.

Further, recombinant or cell-derived proteins may be used, such as recombinant beta-glucan; bovine immunoglobulin concentrate; bovine superoxide dismutase; formulation comprising fluorouracil, epinephrine, and bovine collagen; recombinant hirudin (r-Hir), HIV-1 immunogen; recombinant human growth hormone recombinant EPO (r-EPO); gene-activated EPO (GA-EPO); recombinant human hemoglobin (r-Hb); recombinant human mecasermin (r-IGF-1); recombinant interferon α; lenograstim (G-CSF); olanzapine; recombinant thyroid stimulating hormone (r-TSH); and topotecan.

Still further, the following listing of peptides, proteins, and other large molecules may also be used, such as interleukins 1 through 18, including mutants and analogues; interferons a, y, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-a-y-globulin; superoxide dismutase (SOD); and complement factors, and biologically active analogs, fragments, and derivatives of such factors, for example, growth factors.

Members of the transforming growth factor (TGF) supergene family, which are multifunctional regulatory proteins, may be incorporated in a polymer matrix of the present invention. Members of the TGF supergene family include the beta transforming growth factors (for example, TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (1GF)), (for example, lnhibin A, lnhibin B), growth differentiating factors (for example, GDF-1); and Activins (for example, Activin A, Activin B, Activin AB). Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

In accordance with some embodiments, the TT compounds of the present invention can incorporate and/or include a detectable moiety.

By “detectable label(s) or moieties” is meant a composition that when linked to a molecule of interest renders the latter detectable, via spectroscopic, photochemical, biochemical, immunochemical, or chemical means. Suitable dyes include any commercially available dyes such as, for example, 5(6)-carboxyfluorescein, IRDye 680RD maleimide or IRDye 800CW, Coumarin 6 (C6), Nile Red, Rose Bengal lactone (Rose), and IR-780 iodide (IR-780), ruthenium polypyridyl dyes, and the like. Detectable label(s) or moieties also means useful labels such as radioactive isotopes, magnetic beads, metallic beads, colloidal particles, fluorescent dyes, electron-dense reagents, enzymes (for example, as commonly used in an ELISA), biotin, digoxigenin, or haptens. Specific radioactive labels include most common commercially available isotopes including, for example, ³H, ¹¹C, ¹³C, ¹⁵N, ¹⁸F, ¹⁹F, ¹²³I, ¹²⁴I, ¹²⁵I, ¹³¹I, ⁸⁶Y, ⁸⁹Zr, ¹¹¹In, ^{94m}Tc, ^{99m}Tc, ⁶⁴Cu and ⁶⁸Ga.

Buffers, acids and bases may be incorporated in the compositions to adjust pH. Agents to increase the diffusion distance of agents released from the composition may also be included.

Therapeutic formulations of the product may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

The compositions can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

Isotonicifiers are present to ensure physiological isotonicity of liquid compositions of the instant invention and include polhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Stabilizers can be present in the range from 0.1 to 10,000 w/w per part of compound.

Additional miscellaneous excipients include bulking agents, (e.g., starch), chelating agents (e.g., EDTA), antioxidants (e.g., ascorbic acid, methionine or vitamin E) and co-solvents.

The formulations to be used for in vivo administration must be sterile. That can be accomplished, for example, by filtration through sterile filtration membranes. For example, the formulations of the present invention may be sterilized by filtration.

The compounds and compositions of the present invention will be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the tubustecans to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease. For example, a treatment of interest can increase the use of a joint in a host, based on baseline of the injured or diseases joint, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In another embodiment, an effective amount of a therapeutic or a prophylactic agent of interest reduces the symptoms of a disease, such as a symptom of arthritis by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. Also used herein as an equivalent is the term, “therapeutically effective amount.”

The dose of the compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of the pharmaceutical compositions of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the dose of the pharmaceutical compositions of the present invention can be at a concentration from about 1 nM to about 10,000 nM, preferably from about 10 nM to about 5,000 nM, more preferably from about 100 nM to about 500 nM.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

In accordance with an embodiment of the present invention, the medicament for treating a disease in a subject can encompass many different formulations known in the pharmaceutical arts, including, for example, intravenous and sustained release formulations. With respect to the inventive methods, the disease can include cancer. Cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

In certain embodiments, the subject compositions comprise about 1% to about 75% or more by weight of the total composition, alternatively about 2.5%, 5%, 10%, 20%, 30%, 40%, 50%, 60% or 70%, of a biologically active agent.

Specific examples of useful biologically active agents the above categories include: (a) anti-neoplastics such as androgen inhibitors, antimetabolites, cytotoxic agents, and immunomodulators.

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

In accordance with some embodiments, the Tubustecan compounds of the present invention can have other biologically active agents encapsulated or incorporated into them.

“Incorporated,” “encapsulated,” and “entrapped” are art-recognized when used in reference to a therapeutic agent, dye, or other material and a polymeric composition, such as a composition of the present invention. In certain embodiments, these terms include incorporating, formulating or otherwise including such agent into a composition that allows for sustained release of such agent in the desired application. The terms may contemplate any manner by which a therapeutic agent or other material is incorporated into a polymer matrix, including, for example, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

For example, a solution of a compound or drug of interest can be added to a solution comprising Tubustecans in nanotubular form and allowed to incorporate into the compounds over a course of hours, days or weeks.

More specifically, the physical form in which any therapeutic agent or other material is encapsulated in the Tubustecans may vary with the particular embodiment. For example, a therapeutic agent or other material may be first encapsulated in a microsphere and then combined with the Tubustecans in such a way that at least a portion of the microsphere structure is maintained. Alternatively, a therapeutic agent or other material may be sufficiently immiscible in the Tubustecans of the invention that it is dispersed as small droplets, rather than being dissolved in the polymer. Any form of encapsulation or incorporation is contemplated by the present invention, in so much as the sustained release of any encapsulated therapeutic agent or other material determines whether the form of encapsulation is sufficiently acceptable for any particular use.

In accordance with one or more embodiments, the present invention provides methods for administration of one or more biologically active agents to a cell or population of cells comprising administering to the subject an effective amount of at least one or more compounds described above.

In accordance with one or more embodiments, the present invention provides methods for administration of one or more biologically active agents to a cell or population of cells comprising administering to the subject an effective amount of at least one or more compositions described above.

In accordance with one or more embodiments, the present invention provides methods for administration of one or more biologically active agents to a cell or population of cells comprising administering to the subject an effective amount of at least one or more compositions described above, and at least one additional biologically active agent.

The “therapeutically effective amount” of the pharmaceutical compositions to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as cancer.

In accordance with another embodiment, the present invention provides methods of treating cancer in a subject comprising administering to the mammal a therapeutically effective amount of the composition of the present invention sufficient to slow, stop or reverse the cancer in the subject.

In accordance with an embodiment, the present invention provides a pharmaceutical composition comprising a therapeutically effective amount of the compositions described herein, for use in a medicament, preferably for use in treating a proliferative disease in a subject.

In accordance with a further embodiment, the present invention provides a pharmaceutical composition comprising a therapeutically effective amount of the compositions described herein, for use in a medicament, preferably for use in treating a tumor in a subject sufficient to slow, stop or reverse the growth of the tumor in the subject.

In accordance with still another embodiment, the present invention provides pharmaceutical composition comprising a therapeutically effective amount of the compositions described herein, for use in a medicament, preferably for use in treating cancer in a subject sufficient to slow, stop or reverse the cancer in the subject.

In another embodiment, the term “administering” means that at least one or more pharmaceutical compositions of the present invention are introduced into a subject, preferably a subject receiving treatment for a disease, and the at least one or more compositions are allowed to come in contact with the one or more disease related cells or population of cells.

As used herein, the term “treat,” as well as words stemming therefrom, includes diagnostic and preventative as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Generally, the ingredients are supplied either separately or mixed together in unit dosage form, for example, as a dry lyophilized powder or water-free concentrate in a sealed container, such as an ampule or sachet indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided, for example, in a kit, so that the ingredients may be mixed prior to administration.

An article of manufacture containing materials useful for the treatment of the disorders described above is provided. The article of manufacture comprises a container and a label. Suitable containers include, for example, bottles, vials, syringes and test tubes. The containers may be formed from a variety of materials such as glass or plastic. The container holds a composition which is effective for preventing or treating, for example, a wound or a joint disease and may have a sterile access port (for example, the container may be a vial having a stopper pierceable by a hypodermic injection needle). The label on or associated with the container indicates that the composition is used for treating the condition of choice. The article of manufacture may further comprise a second container comprising a pharmaceutically acceptable buffer, such as phosphate-buffered saline, Ringer's solution and dextrose solution. It may further include other materials desirable from a commercial and user standpoint, including buffers, diluents, filters, needles, syringes and package inserts with instructions for use.

In accordance with one or more embodiments, the present invention provides methods for making the compounds and compositions described above.

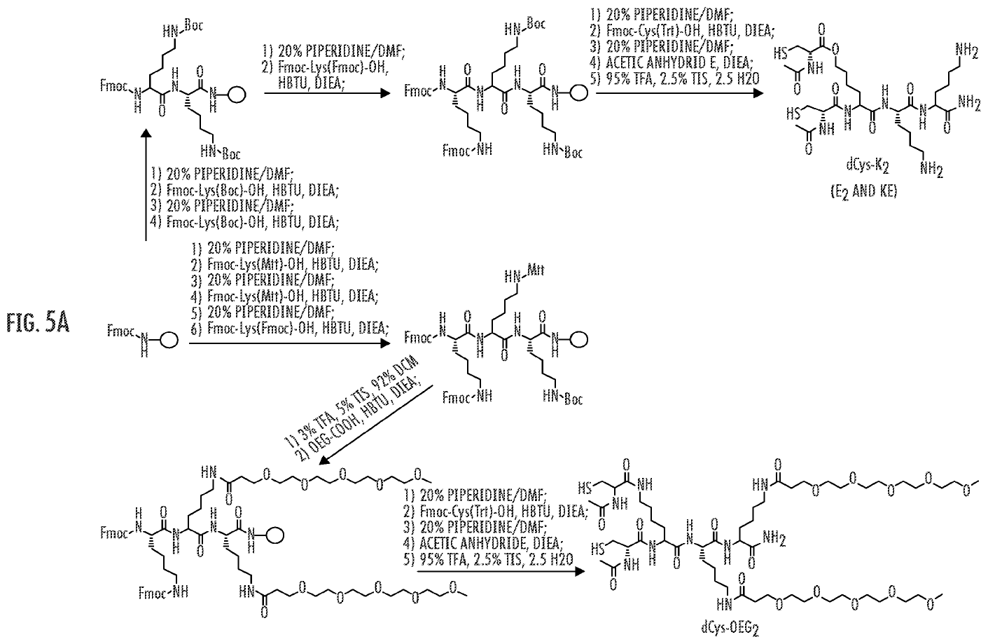

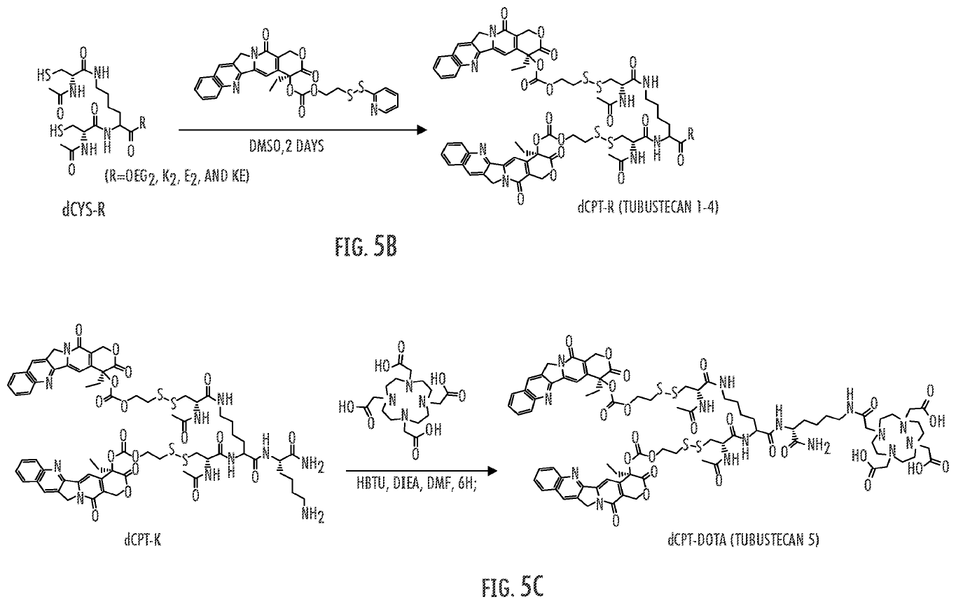

FIG. 5A illustrates the manual synthetic protocols used for synthesizing some examples of peptidic precursors. The three peptides dCys-K₂, dCys-E₂, and dCys-KE used similar synthetic procedures by sequentially adding amino acids. Fmoc-Lys(Fmoc)-OH was introduced as a branching motif to yield a dual-functional reaction point. Following Fmoc removal, Fmoc-Cys(Trt)-OH or another amino acid of interest is conjugated onto each N-terminus to furnish thiol groups for drug conjugation. All Fmoc deprotections are performed using a 20% 4-methylpiperidine in DMF solution for 15 min and repeated once. The amino acid coupling was performed after Fmoc deprotection by adding a mixture of Fmoc-amino acids, HBTU and DIEA (4:4:6 molar equiv to resin) in DMF for 2 h.

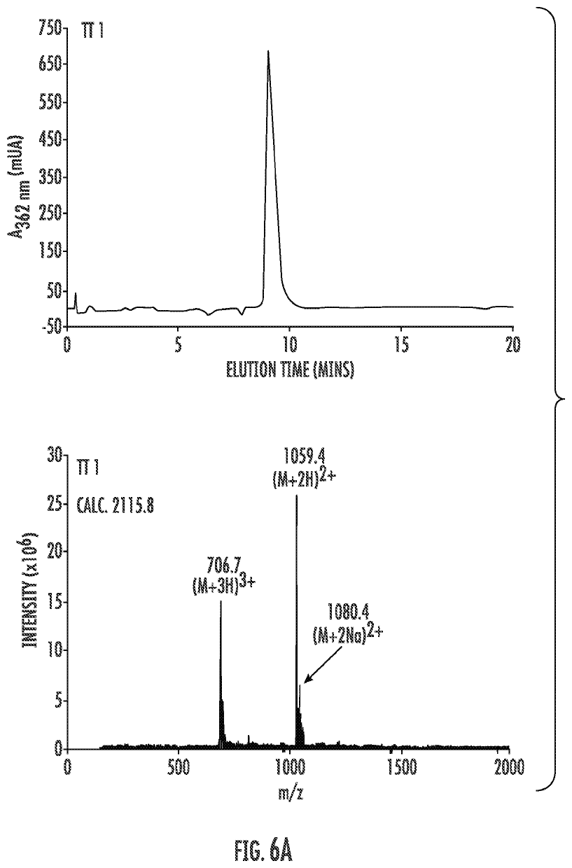

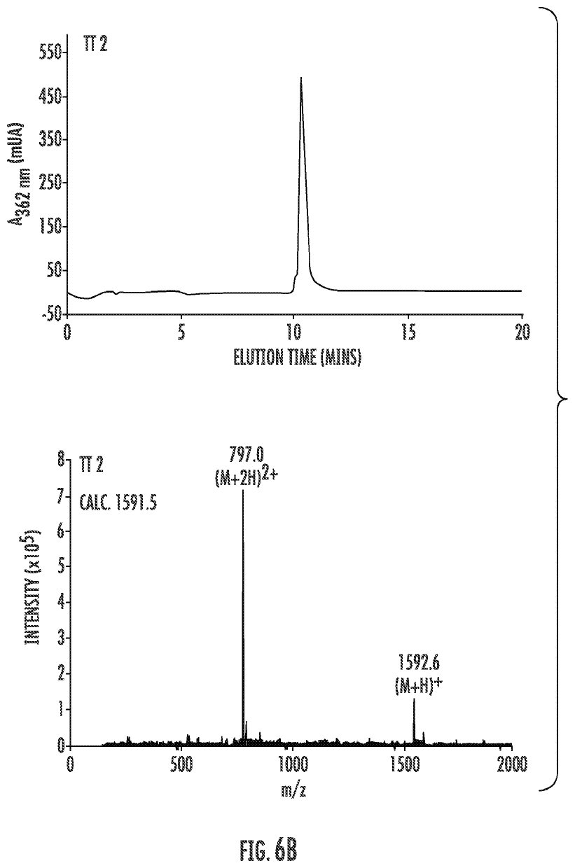

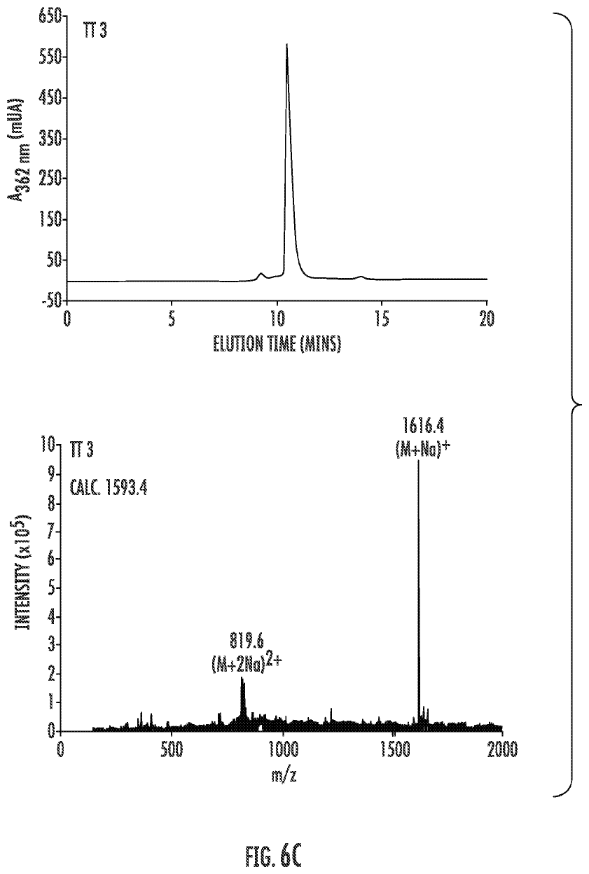

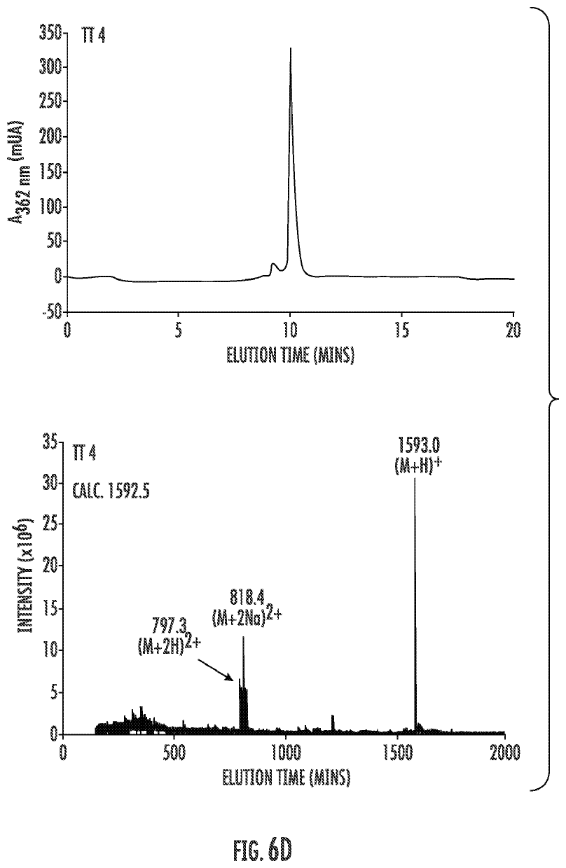

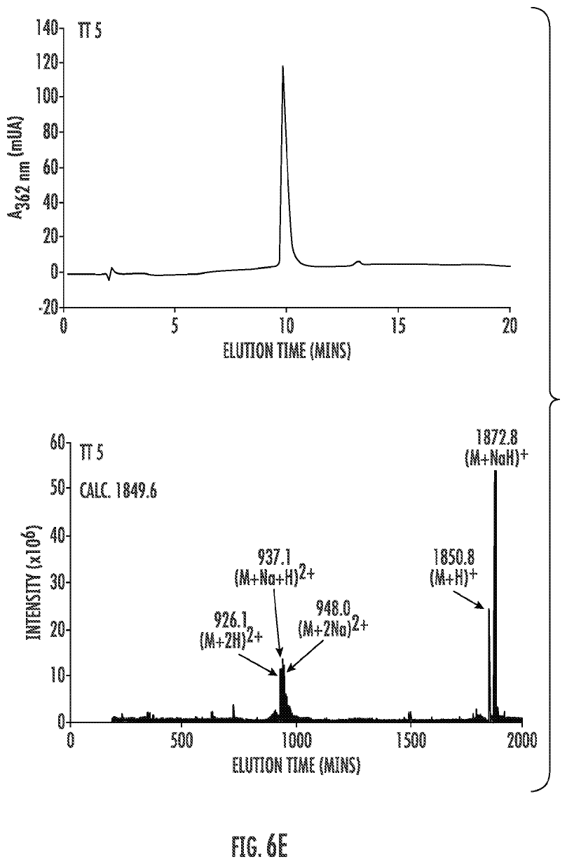

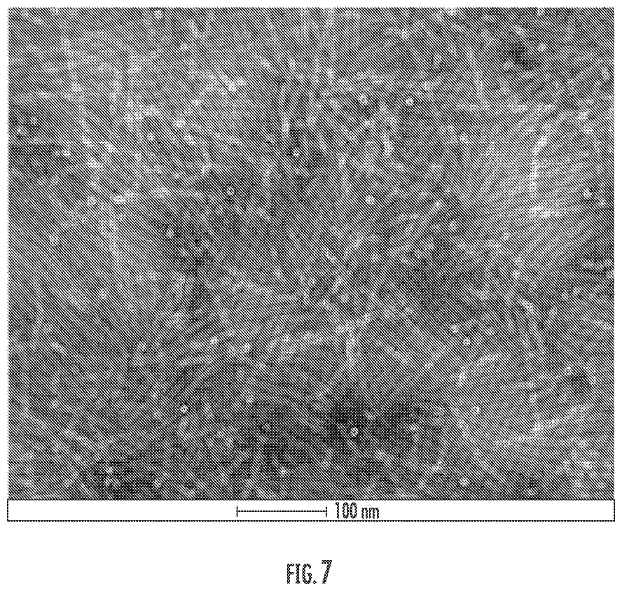

The synthesis of functional Tubustecans is carried out by mixing CPT-etcSS-Pyr, or another drug and linker of interest, and the corresponding crude peptides synthesized above in N2-purged DMSO with a molar ratio of 3:1 (FIG. 5B). After reacting for 2 days, the mixture was diluted with 0.1% TFA in acetonitrile/water and purified by preparative RP-HPLC. Collected fractions were analyzed by ESI-MS (FIG. 6) and the appropriate fractions were combined, concentrated, and lyophilized on a FreeZone −105° C. 4.5 L freeze dryer.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Materials and Methods

Fmoc amino acids (except Fmoc-Lys(Fmoc)-OH) and coupling reagents (HBTU or HATU) were sourced from Advanced Automated Peptide Protein Technologies (AAPPTEC, Louisville, Ky., USA). Rink amide MBHA resin and Fmoc-Lys(Fmoc)-OH were obtained from Novabiochem (San Diego, Calif., USA). mPEG4-CH₂CH₂COOH (OEG₅-COOH) was purchased from ChemPep Inc. (Wellington, Fla., USA). 1,4,7,10-tetraazacyclododecane-1,4,7,10-tetraacetic acid (DOTA) was sourced from Strem Chemicals, Inc. (Newburyport, Mass., USA). Camptothecin was purchased from Chem-Impex International Inc. (Wood Dale, Ill., USA) and all other reagents were sourced from Sigma-Aldrich (St. Louis, Mo.) or VWR (Radnor, Pa., USA), unless otherwise stated.

RP-HPLC was performed on a Varian ProStar Model 325 HPLC (Agilent Technologies, Santa Clara, Calif.). Preparative separations utilized a Varian PLRP-S column (100 Å, 10 μm, 150×25 mm), while analytical HPLC used a Varian Pursuit XRs C18 column (5 μm, 150×4.6 mm). Water and acetonitrile containing 0.1% v/v TFA were used as the mobile phase. Purified fractions were lyophilized using a FreeZone −105° C. 4.5 L freeze dryer (Labconco, Kansas City, Mo.). ESI-MS mass spectrometric data was acquired on a Finnigan LDQ Deca ion-trap mass spectrometer (Thermo-Finnigan, Waltham, Mass.).

Synthesis of Self-Assembling Prodrug Tubustecans (TTs)

All peptide sequences were synthesized on Rink Amide MBHA resins using standard 9-fluorenylmethoxycarbonyl (Fmoc) solid phase synthesis techniques on a 0.25 mmol scale. FIG. 5A illustrates the manual synthetic protocols used for synthesizing peptidic precursors. In one embodiment, three peptides dCys-K₂, dCys-E₂, and dCys-KE used similar synthetic procedures by sequentially adding amino acids. Fmoc-Lys(Fmoc)-OH was introduced as a branching motif to yield a dual-functional reaction point. Briefly, Rink Amide MBHA resins were swell in DCM and Fmoc groups were deprotected using a 20% 4-methylpiperidine in DMF solution. The amino acids, for example Fmoc-Lys(Mtt)-OH, were conjugated onto the resins by adding Fmoc-Lys(Mtt)-OH/HBTU/DIEA at a ratio of 4:4:6 (molar equiv to resin) in DMF and the mixture was shaken for 2 h. This Fmoc deprotection and amino acid coupling process was repeated to add more amino acids to the peptide chains. The four peptides have 2, 4, 6, and 8 Fmoc-Lys(Mtt)-OHs, respectively. After that, Mtt groups on the lysine side chain were deprotected by adding 3% TFA/5% TIS/92% DCM for 5 min (repeat 5-6 times), and OEGS—COOH was subsequently conjugated onto the peptide by amide formation reaction using OEGS—COOH/HBTU/DIEA at a ratio of 2:2:3 (molar equiv to resin). After another Fmoc removal, Fmoc-Lys(Fmoc)-OH was added as a branching site for further conjugation of the two Fmoc-Cys(Trt)-OHs. In addition, the Fmoc groups on the Cysteines were deprotected and acetylated by adding 20% acetic anhydride/DMF solution with 100 μL DIEA. The peptides were cleaved from the resins by adding a mixture of TFA/TIS/H2O at a ratio of 95:2.5:2.5 and shaking for 3 h. The TFA solution was collected, concentrated, and precipitated in cold diethyl ether. The crude peptides were centrifuged down, washed twice with diethyl ether and dried under vacuum. In the synthesis of dCys-OEG2, two Fmoc-Lys(Mtt)-OH molecules were first loaded onto the resin to allow selective deprotection and functionalization of the lysine side chain amino groups. Following Mtt deprotection (3% TFA, 5% TIS, 92% DCM), OEG5-COOH was conjugated onto the side chain of the lysine through amide bond formation in the manner described earlier for the amino acid couplings (FIG. 5A). The synthesis of dCys-K employed the same Fmoc peptide synthesis procedures detailed above.

In the synthesis of dCys-OEG2, two Fmoc-Lys(Mtt)-OH molecules were first loaded onto the resin to allow selective deprotection and functionalization of the lysine side chain amino groups. Following Mtt deprotection (3% TFA, 5% TIS, 92% DCM), OEG5-COOH was conjugated onto the side chain of the lysine through amide bond formation in the manner described earlier for the amino acid couplings (FIG. 5A). The synthesis of dCys-K employed the same Fmoc peptide synthesis procedures detailed above.

The synthesis of functional TTs was carried out by mixing CPT-etcSS-Pyr and the corresponding crude peptides synthesized above in N2-purged DMSO with a molar ratio of 3:1 (FIG. 5B). After reacting for 2 days, the mixture was diluted with 0.1% TFA in acetonitrile/water and purified by preparative RP-HPLC. All separations were performed using a flow rate of 20 mL/min for 25 mins in total, monitoring at 362 nm. The mobile phase gradient began at 15% MeCN, increasing to 80% MeCN over 20 min, and then holding for 2 min before returning to initial conditions over 3 min. Collected fractions were analyzed by ESI-MS (FIG. 6) and the appropriate fractions were combined, concentrated, and lyophilized on a FreeZone −105° C. 4.5 L freeze dryer. The powders obtained were then re-dissolved, calibrated, and aliquotted into cryo-vials before re-lyophilization. The synthesis of TT 5 was carried out by reacting DOTA with free amine group on the lysine side chain of dCPT-K and purified again by HPLC using methods mentioned above.

The purity of the conjugates was proven by analytical RP-HPLC using the following conditions: the flow rate was 1 mL/min, with the mobile phase gradient starting from 5% MeCN (with 0.1% TFA), increasing to 95% MeCN (with 0.1% TFA) over 15 min, and then holding for 1 min before returning to the initial conditions over 4 min; the monitored wavelength was 362 nm (FIG. 6). The HPLC was equipped with a Varian Pursuit XRs C18 column (5 μm, 150×4.6 mm) for analytical use and a Varian PLRP-S column (100 Å, 10 μm, 150×25 mm) for separation purpose. The analytical method was a flow rate of 1 mL/min for 20 mins from 10% acetonitrile to 70% acetonitrile and the preparative method was a flow rate of 20 mL/min for 25 mins from 10% acetonitrile to 40% acetonitrile. The proper fractions were collected and analyzed by ESI-MS using a Finnigan LDQ Deca ion-trap mass spectrometer (Thermo-Finnigan, Waltham, Mass.) and analytical HPLC again (Fig. S1 and S3-S6). The final products were concentrated and lyophilized using a FreeZone −105° C. 4.5 L freeze dryer (Labconco, Kansas City, Mo.).

The purified peptide precursors were further reacted with CPT-etcSS-Pyr prodrug in N2-purged DMSO over 2 days at the prodrug/peptide ratio of 2.4:1 (1, 2). After the reaction, the separation of the targeted molecules was performed by preparative HPLC again with a flow rate of 20 mL/min for 30 mins from 25% acetonitrile to 65% acetonitrile and monitored at 362 nm. The proper fractions were collected and analyzed by ESI-MS and analytical HPLC. Molecular masses were determined using ESI-MS (FIG. 6).

Calibration of the TT Concentration

The concentrations of purified TTs were determined by calculating the amount of free CPT produced from the prodrugs upon reduction of the disulfide linker. 25 μL stock solution of the corresponding prodrug in MeCN/H2O (1:1) was diluted to 50 μL by adding 25 μL 1 M TCEP solution in MeCN/H2O (1:1) and mixing via periodic vortexing. 25 μL of the solution was then injected onto the HPLC (so as to completely fill the 20 μL loop), measuring the area under the peak of free CPT at 362 nm. The CPT concentration of treated conjugates was obtained by comparison with the standard calibration curve of CPT. The TT concentration was calculated based on the applied dilutions and number of CPT molecules. Finally, the stock solution was diluted to 200 μM, 400 μM, 1 mM and 5 mM according to the calibrated concentration and aliquotted into cryo-vials before re-lyophilization.

CPT Standard calibration curve: y=17.095x+59.358, where y is the area under the cruve and x is the concentration of CPT (μM).

Transmission electron microscopy (TEM) protocol

About 200 μM stock solutions of corresponding TTs in water were prepared by dissolution of the lyophilized powders. After aging overnight, TEM samples were prepared by depositing 7 μL of the appropriate solution onto a carbon-coated copper grid (Electron Microscopy Services, Hatfield, Pa., USA), wicking away the excess solution with a small piece of filter paper. Next, 7 μL of a 2 wt % uranyl acetate aqueous solution was deposited on the surface for 30 seconds, wicking away the excess solution with filter paper. The grids were then air-dried overnight at room temperature prior to imaging. Bright-field TEM imaging was performed using an FEI Tecnai 12 TWIN Transmission Electron Microscope operated at an acceleration voltage of 100 kV. All TEM images were acquired by a SIS Megaview III wide-angle CCD camera or 16 bit 2K×2K FEI Eagle bottom mount camera.

Cryogenic Transmission Electron Microscopy (Cryo-TEM) Protocol

Cryo-TEM imaging was performed using higher sample concentrations of 800 μM (compared with 200 μM for conventional TEM imaging). Extended imaging times can result in damage to the vitreous ice film caused by the electron beam and so higher concentrations can allow a more rapid visualization that reduces this likelihood. About 6 μL of the appropriate solution was dropped onto a lacey carbon-film-supported TEM copper grid (Electron Microscopy Services, Hatfield, Pa., USA). All the TEM grids used for cryo-TEM imaging were pretreated with plasma air to render the lacey carbon film hydrophilic. A thin film of the sample solution was produced using a Vitrobot with a controlled humidity chamber (FEI). After loading of the sample solution, the lacey carbon grid was blotted using preset parameters and plunged instantly into a liquid ethane reservoir precooled by liquid nitrogen. The vitrified samples were then transferred to a cryo-holder and cryo-transfer stage that was cooled by liquid nitrogen. To prevent sublimation of vitreous water, the cryo-holder temperature was maintained below −170° C. during the imaging process. All images were recorded by a Tecnai 12 microscope with a cryo-holder, and the images were acquired by a 16 bit 2K×2K FEI Eagle bottom mount camera.

Circular Dichroism (CD) Spectroscopy of TT 1 Nanotubes

All CD spectra were recorded on a Jasco J-710 spectropolarimeter (JASCO, Easton, Md., USA) from 190 to 480 nm using 1 mm path length quartz UV-Vis absorption cell (Thermo Fisher Scientific, Pittsburgh, Pa., USA). TT 1 solution of 200 μM was measured and the obtained spectrum was converted from ellipticity (mdeg) to molar ellipticity (deg·cm² dmol⁻¹). The background spectrum of the solvent was acquired and subtracted from the sample spectrum.

CD Measurements of TT Solutions

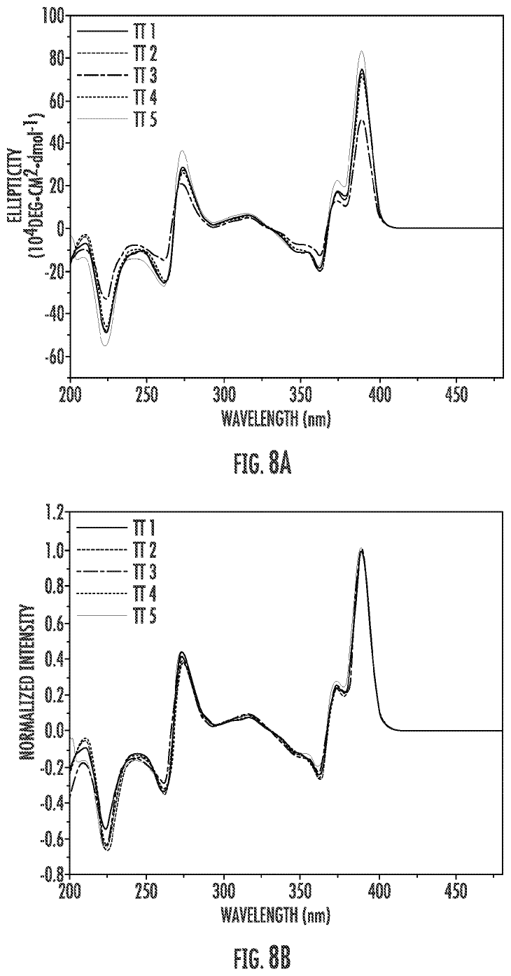

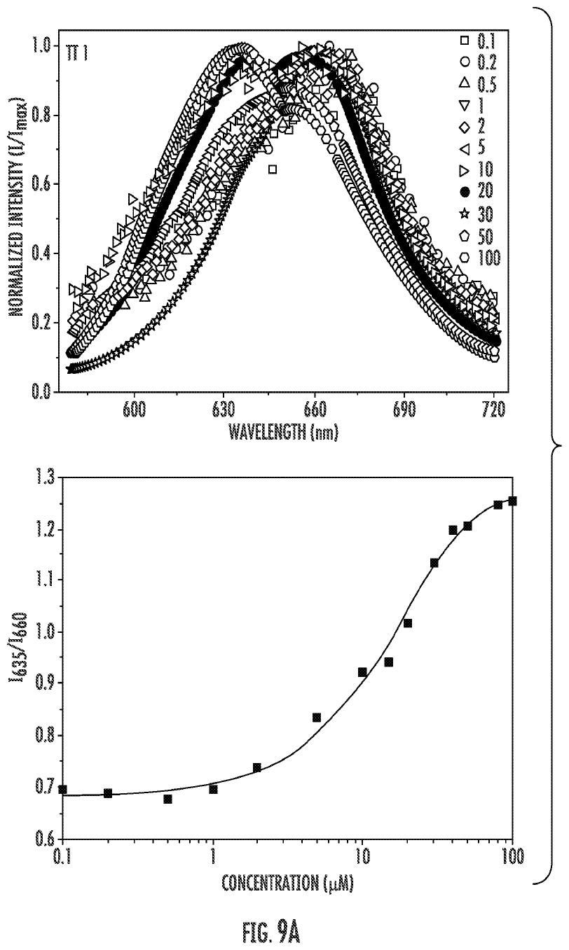

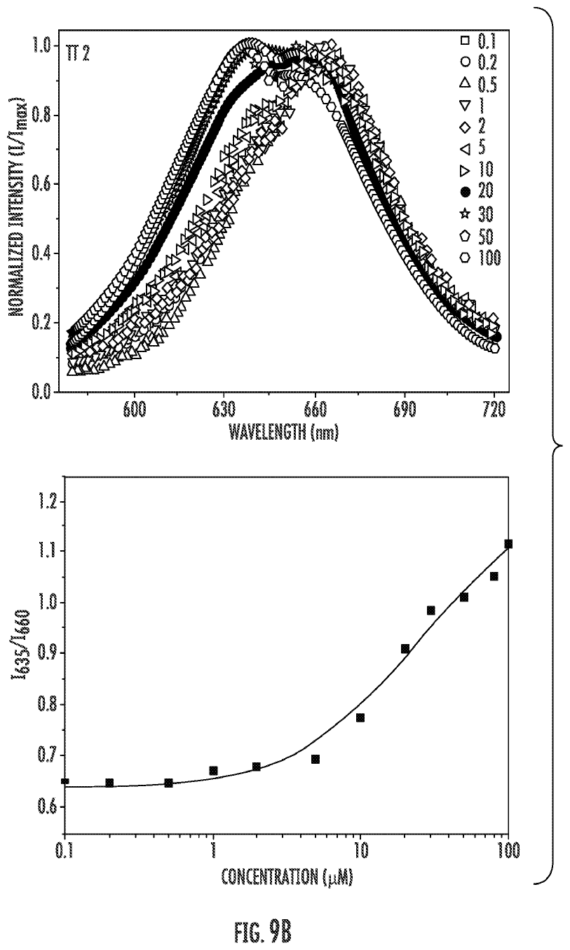

Various TT solutions of 200 μM were measured according to the protocols described in section 5. Collected data was converted from ellipticity (mdeg) to molar ellipticity (deg·cm²·dmol⁻¹) and is shown in FIG. 8A. CD spectra for all five TT molecules were further normalized by the maximum intensity to verify the similarity of their CD spectra (FIG. 8B).

Critical Micellization Concentration (CMC) Measurement of TTs Via Nile Red Encapsulation

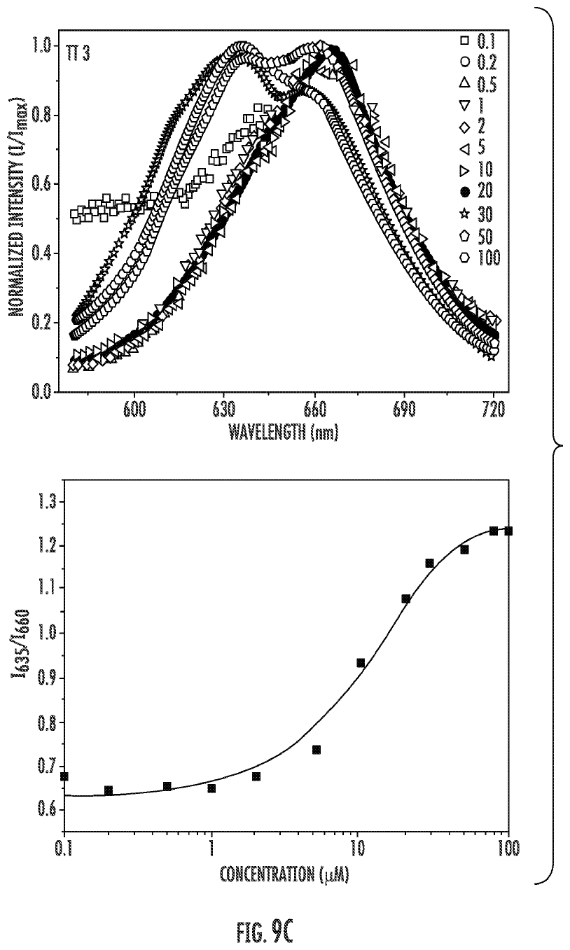

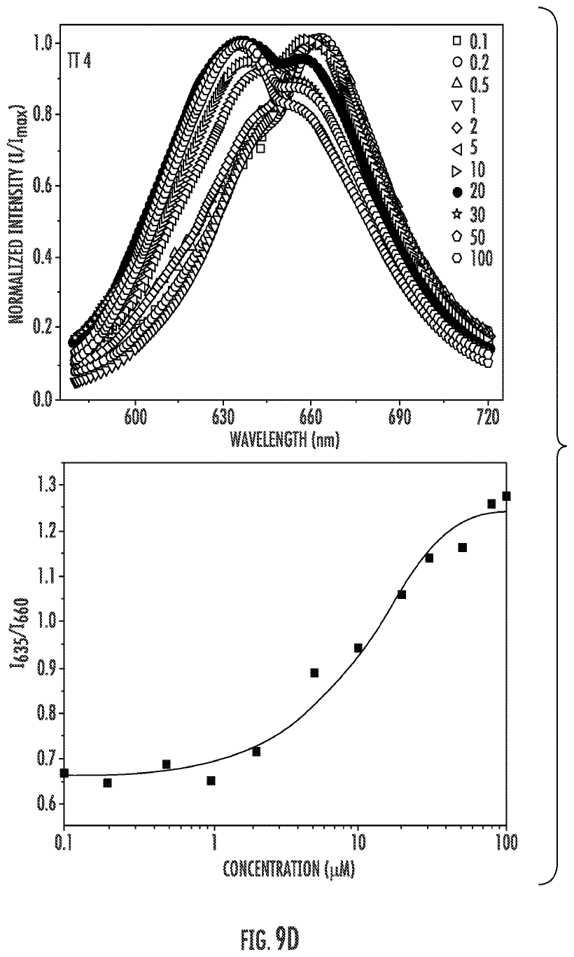

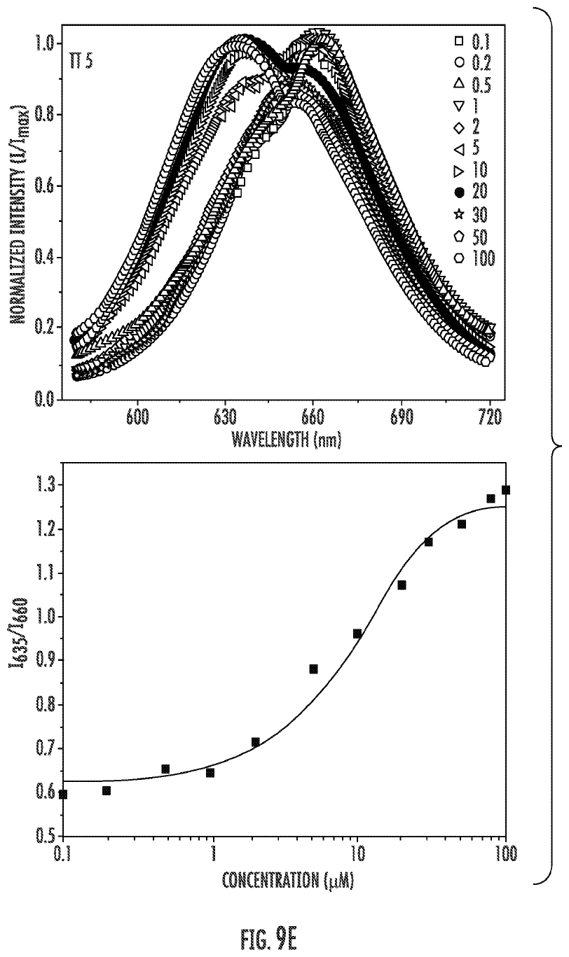

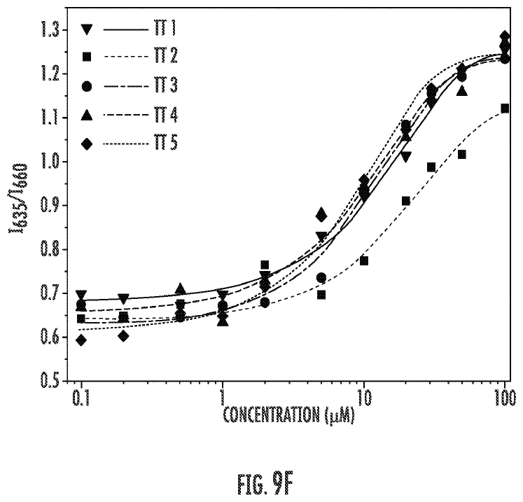

Nile Red is a hydrophobic, solvatochromic dye that fluoresces intensely upon exposure to hydrophobic environments compared with its strongly quenched and red-shifted fluorescence in aqueous environments. The CMC of the TTs was determined by incubating these molecules at various concentrations with a fixed content of Nile Red. 10 μL of a 1 mM Nile Red stock solution in acetone was added to each microcentrifuge tube to be used, with the acetone allowed to evaporate in a dark area. TT solutions of various concentrations were subsequently added to the Nile Red containing tubes and equilibrated overnight. The emission spectrum for each sample was then recorded on a Fluorolog spectrofluorometer (Horiba Jobin Yvon Inc., Edison, N.J.), acquiring between 580 and 720 nm with an excitation wavelength of 550 nm. The ratio of intensity at 635 nm (emission maximum of the dye in hydrophobic environment) to that at 660 nm (emission maximum in aqueous conditions) was then plotted against the concentration of each TT, which shows a transition in the data when the TT concentration exceeded the CMC (FIG. 9).

Zeta Potential Measurement

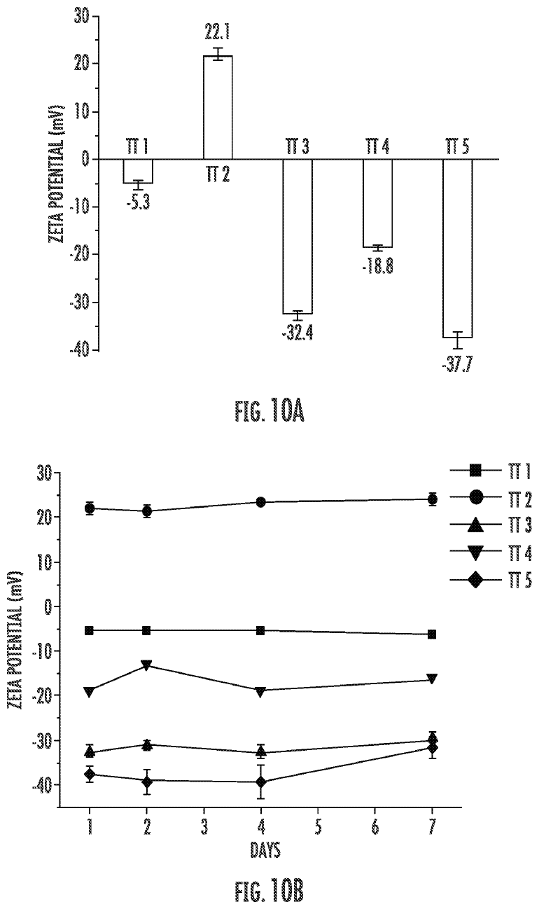

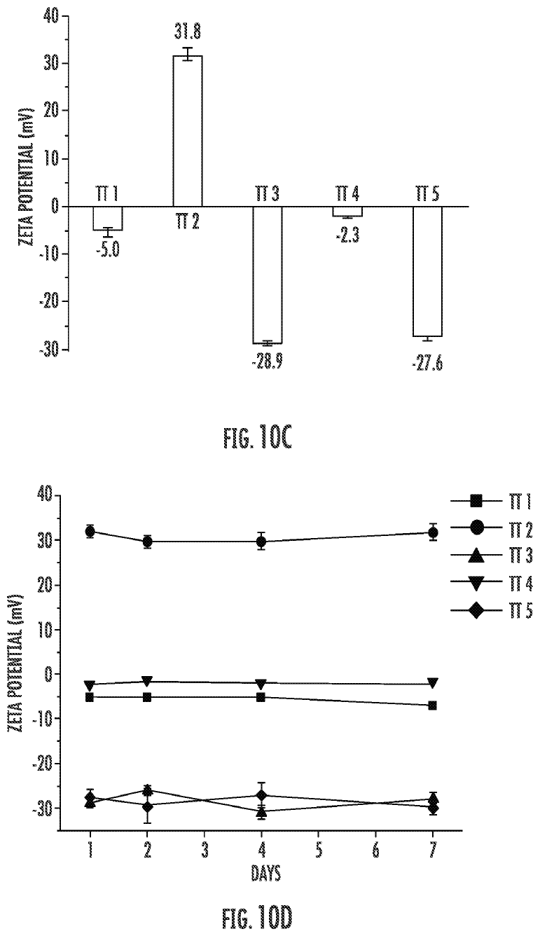

TT solutions of 200 μM in 1×-DPBS buffer (pH=7.4) were prepared by directly mixing identical volumes of a 400 μM aqueous TT solution and 2×-DPBS buffer (pH=7.4). In some embodiments, solutions at a concentration of 2 mM in PBS buffer (pH=7.4) were prepared and aged overnight prior to zeta potential measurement. TT solutions of 200 μM in 1×-DPBS buffer (pH=5.0) were prepared by mixing identical volumes of a 400 μM aqueous TT solution and 2×-DPBS buffer (pH=5.0), which was pretreated with 6 M HCl. The zeta potential measurements were performed on a Zetasizer Nano ZS90 (Malvern Instruments Ltd., UK). The prepared solutions were loaded in capillary cells and equilibrated for 2 min prior to measurement. The average values and their standard deviations are calculated from three replicate measurements. The zeta potential of the assembled structures was obtained by measuring the electrophoretic movement of the nanostructures under the applied electric field, where the movement velocity is determined by phase analysis light scattering. Variations of the zeta potential value over time were determined by measuring the solutions at different aging time points (1, 2, 4 and 7 days) and are plotted in FIG. 10.

Drug Release from TT 1 Nanotubes

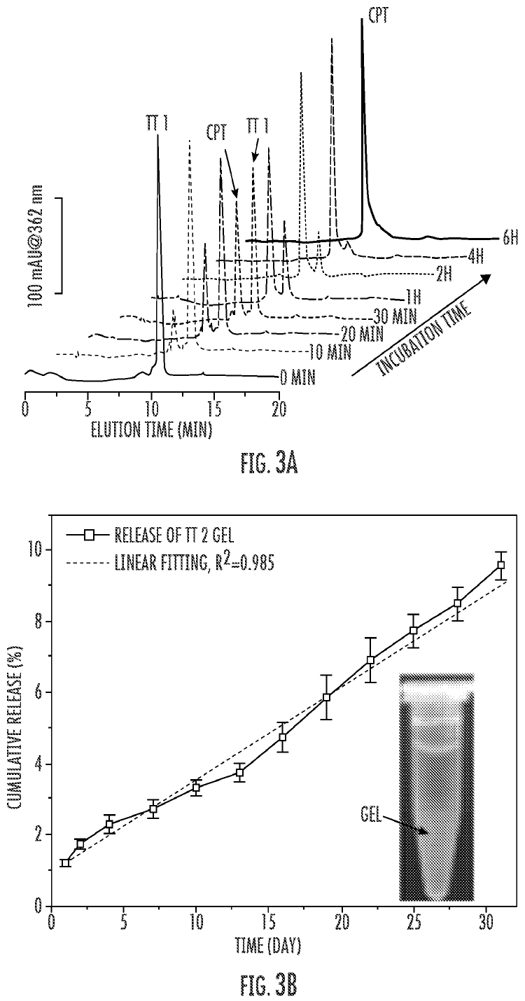

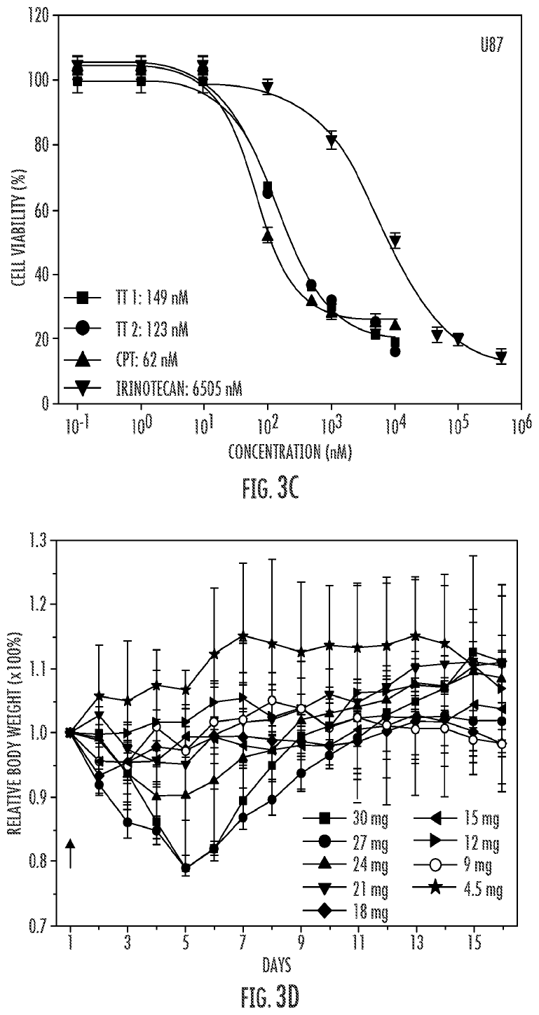

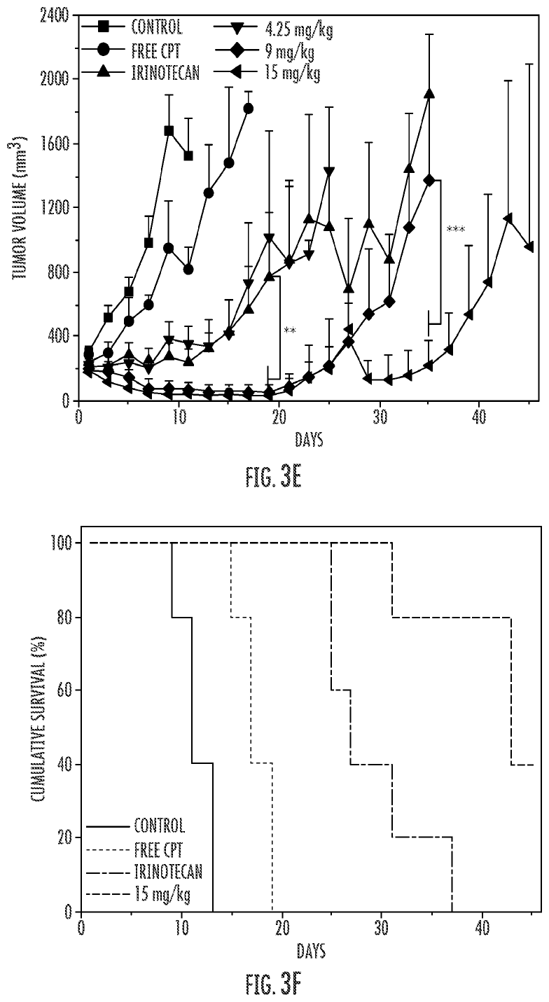

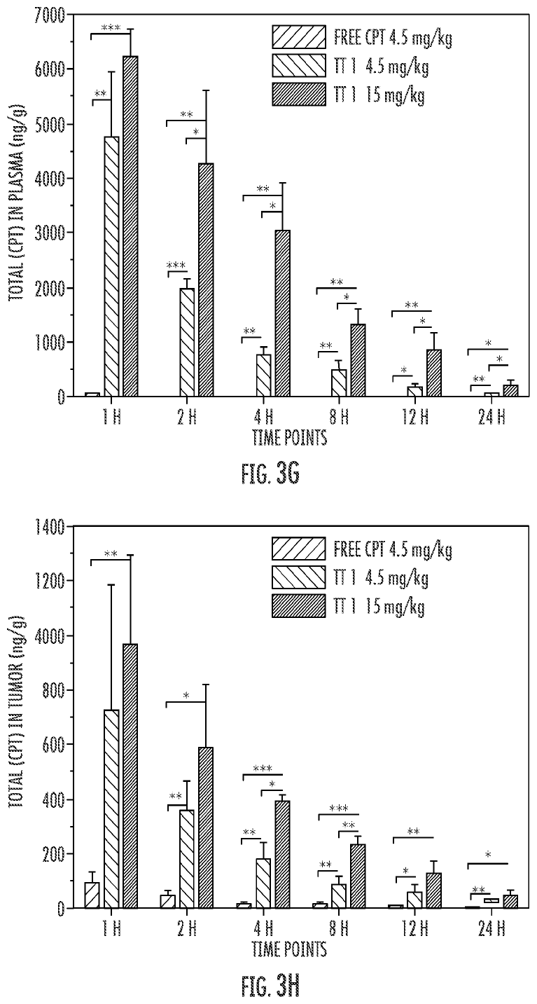

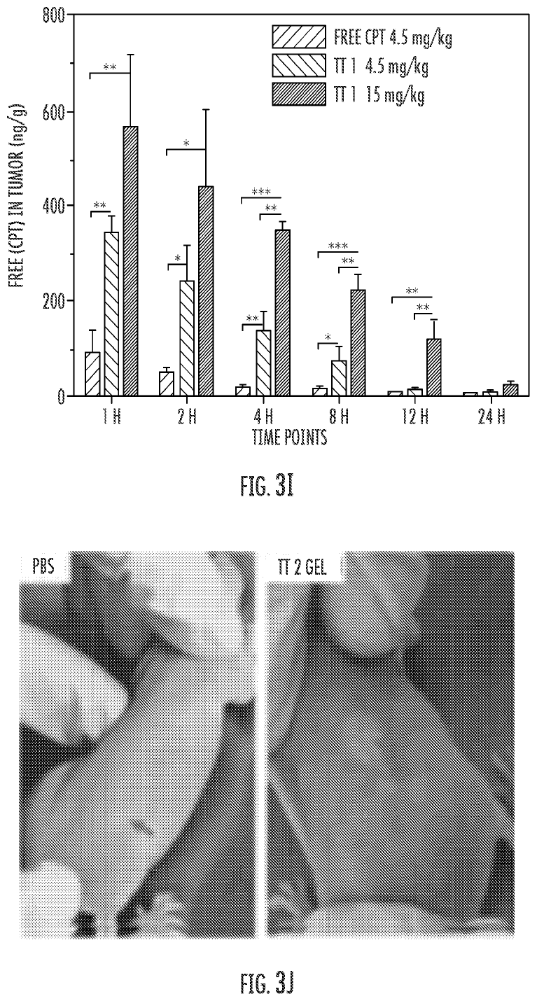

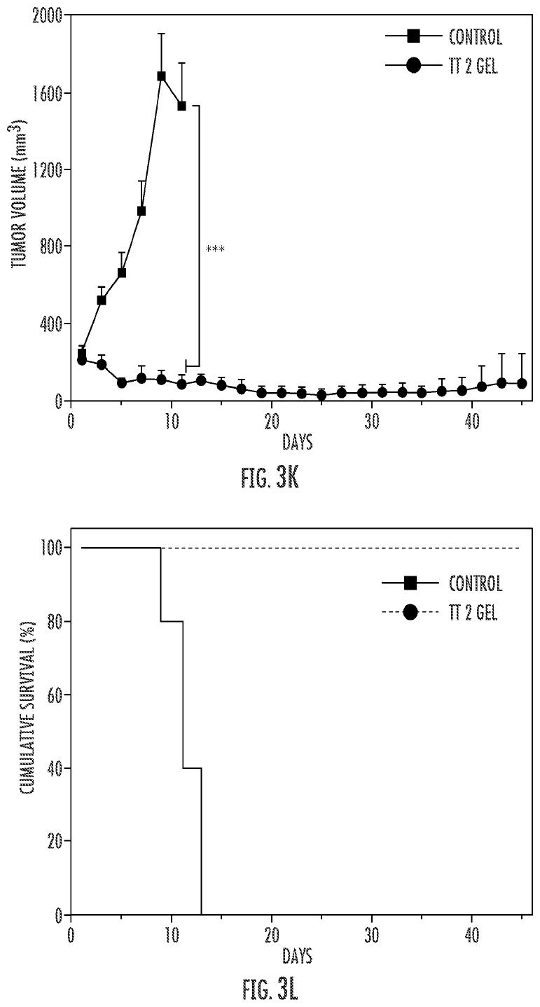

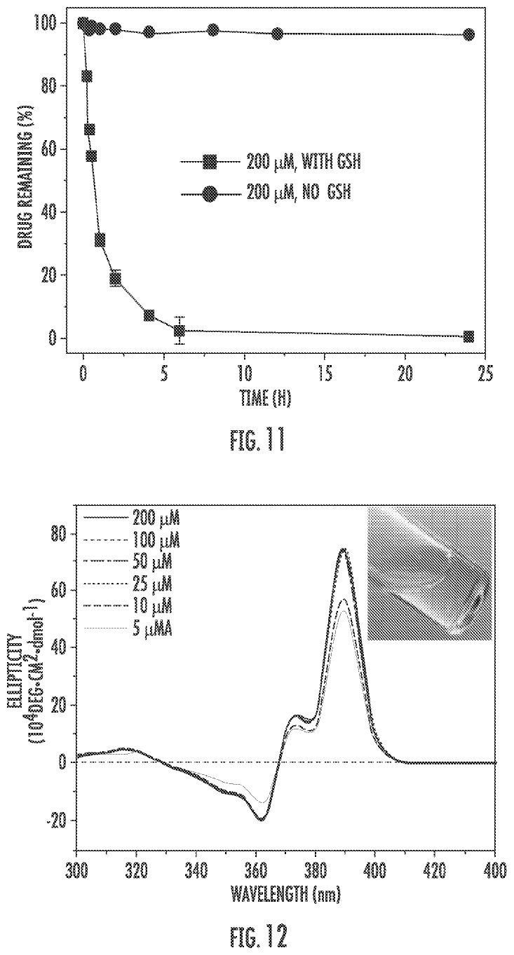

The release of free CPT from TT 1 nanotubes was evaluated in the presence or absence of GSH. Stock solutions of 400 μM of TT 1 in deionized water were prepared and diluted to 200 μM with 20 mM PBS buffer with or without GSH (20 mM). Three replicate experiments were prepared together for both conditions. The solutions were incubated at 37° C. and samples were collected at 0 min, 10 min, 20 min, 30 min, 1 h, 2 h, 4 h, 6 h, 8 h, 12 h and 24 h. For each collected sample, the reductive release was halted by acidification of the solution through the addition of 0.2 μL of 2M HCl. Samples were then frozen with liquid nitrogen and stored at −30° C. until analysis. The amount of released CPT was monitored by RP-HPLC using the following conditions: Varian Pursuit XRs C18 (5 μm, 150×4.6 mm); 362 nm detection wavelength; 1 ml/min flow rate; the gradient began at 90% of mobile phase A (0.1% aqueous TFA) and 10% of mobile phase B (acetonitrile containing 0.1% TFA) increasing to 90% mobile phase B by 15 min and held for another 1 min before decreasing to the initial solvent composition at 20 minutes. Selected time points were characterized and data were plotted as a percentage of the total expected CPT concentration. It was found that 80% of the conjugated CPT molecules were released within 2 h in the presence of GSH, reaching almost 100% by 6 h, while only a slight amount (less than 10%) of the conjugates had degraded in 24 h without GSH (FIG. 11). Detailed HPLC traces at different time points were also summarized, clearly demonstrating the release trend of free CPT (FIG. 3A).

Physical Stability of Non-Ionic TT 1 Nanotubes

The stability of non-ionic TT 1 nanotubes upon dilution in cell medium was evaluated by recording the CD spectrum for a series of prepared dilutions. Phenol red-free DMEM (Mediatech) containing 10% fetal bovine serum (FBS, Invitrogen) and 1% antibiotics (Invitrogen) was used as the cell medium solution, thereby avoiding any potential spectroscopic interferences that would otherwise be caused by the dye. A stock solution of TT 1 nanotube was prepared at a concentration of 1 mM in water and aged overnight. The aged solution was then diluted to 200 μM with cell medium. Further dilutions of the 200 μM TT 1 nanotube in cell medium were prepared at 100 μM, 50 μM, 25 μM, 10 μM, and 5 μM concentration. All diluted solutions were incubated for an additional two days before CD measurements were made. All the CD spectra were recorded from 300 to 440 nm using a 1 mm (for 200 μM, 100 μM, and 50 μM) or 10 mm (for 25 μM, 10 μM, and 5 μM) path length quartz cell. The spectra were collected and normalized from ellipticity (mdeg) to molar ellipticity (deg·cm²·dmol⁻¹). No significant changes were observed in the normalized curves, indicating no obvious disassociation of the self-assembled nanotubular structures at concentrations above 25 μM (FIG. 12). The CD spectra of TT 1 solutions at 5 and 10 μM showed slight decreases in intensity with no change to the overall profile, indicating that slight disassociation of nanotubes may have occurred. In addition, the solutions of TT 1 nanotubes in cell medium remained clear after one week, suggesting no obvious aggregation of nanotubes in cell medium (FIG. 12).

Cytotoxicity Studies of TT 1 and TT 2 Against U87 MG Brain Tumor Cell

The human brain cancer cell line U87 MG was a generous gift from Dr. Wirtz (ChemBE, JHU). DMEM (Invitrogen) containing 10% fetal bovine serum (FBS, Invitrogen) and 1% antibiotics (Invitrogen) was used for the culture of the U87 MG cells. Cancer cells were incubated at 37° C. in a humidified incubator (Oasis, Caron, Marietta, Ohio, USA) with an atmosphere of 5% CO2. The cytotoxicities of TT 1 and TT 2 were evaluated using the SRB method. U87 MG cells were seeded onto 96-well plates (5000 cells/well) and allowed to attach overnight. 1 mM aqueous stock solutions of TT 1 and TT 2 were prepared and aged overnight. The stock solutions were then diluted with fresh medium to achieve final CPT concentrations of 0.1, 1, 10, 100, 500, 1000, 5000 and 10000 nM. After dilution, the nanotube-containing media were used to incubate cells immediately. Medium containing the same concentration of free CPT ranging from 0.1 to 10000 nM was also used to incubate the cells, with non-treated cells (solvent only) as the control group. In addition, irinotecan at the concentration of 0.1, 1, 5, 10, 50, 100 and 500 μM was employed as a second control. After 48 h incubation, the cell viability was evaluated using the SRB method according to the manufacturer's protocols (TOX-6, Sigma, St. Louis, Mo.). The results suggested that both TT 1 and TT 2 show an enhanced efficacy against U87 MG cells compared with irinotecan and were even comparable to its parent drug CPT.

Gel Release of TT 2 Nanotube Hydrogel

TT 2 was dissolved in water and hydrogel formation triggered by the addition of 10×-DPBS to give a final TT 2 concentration of 10 mM in 1×-DPBS. 200 μL of the hydrogel was placed at the bottom of centrifuge tube and allowed to re-gel overnight. Three replicate experiments were prepared together and incubated at 37° C. Fresh 1×-DPBS (300 μL) was layered on top of the hydrogel and was refreshed at predetermined time points: 1, 2, 4, 7, 10, 13, 16, 19, 22, 25, 28, and 31 days. The samples were frozen with liquid nitrogen and stored at −30° C. until analysis. The amount of TT 2 in the top DPBS layer was determined by analytical RP-HPLC using the conditions described in the previous paragraph. The cumulative release of TT 2 from its hydrogels was calculated and plotted as a percentage of the total amount of hydrogel. The conjugate was seen to be released almost linearly from the hydrogel with ˜10% of the prodrugs being released over the 31-day period.

In Vivo Animal Studies

Biodistribution studies were performed at the Shanghai Institute of Materia Medica, Chinese Academy of Sciences. Female nude mice (18-22 g) were purchased from the Shanghai Experimental Animal Center (Shanghai). All animal procedures were performed under guidelines approved by the Institutional Animal Care and Use Committee of the Shanghai Institute of Materia Medica, Chinese Academy of Sciences. All other experiments conducted with mice were performed at the Johns Hopkins University (JHU) in accordance with protocols approved by the JHU Institutional Animal Care and Use Committee (IACUC). Female athymic nude mice were obtained from the Charles River and kept at the JHU Animal Care Facility. The animals were acclimatized to the laboratory environment for at least one week prior to the experiments.

Maximum Tolerated Dose (MTD) Determination

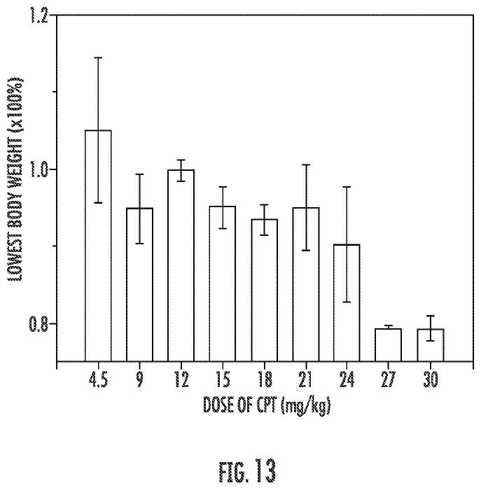

The MTD was determined using healthy female athymic nude mice (Charles River, 12-13 weeks old). A single dose of TT 1 was administered through intravenous injection on day 1 and the body weights of each mouse was recorded every day from day 1 to day 16 (n=3). The dosing volume was determined based upon a ratio of 200 μL for a 20 g mouse and was scaled appropriately according to the actual body weight of the mice. Doses were 54, 36, 30, 27, 24, 21, 18, 15, 12, 9 and 4.5 mg/kg (CPT equivalent) (FIG. 13). The maximum tolerated dose (MTD) was determined by the largest dose that did not result in more than a 20% mean body weight loss or death of an animal in that group. Doses of 54 and 36 mg/kg caused at least one death in each group.

Antitumor Efficacy Study for Systemic Delivery

A total of 2×10⁶ human glioblastoma U87 MG cells were subcutaneously injected into the right shoulder of athymic nude mice (8-9 weeks old). The mice were used for the efficacy study after three to four weeks when the tumor had reached about 190-250 mm³ in size. Mice were randomly divided into six groups (n=5) of non-treated, CPT (4.5 mg/kg), Irinotecan (60 mg/kg), 4.5 mg/kg, 9 mg/kg and 15 mg/kg TT 1. Water insoluble free CPT was dissolved/suspended in a mixture of DMSO/ethanol/PEG-400/water (1:1:2:1). CPT (4.5 mg/kg) and irinotecan (60 mg/kg) were administered by intraperitoneal injection, while freshly prepared TT 1 solutions of various doses were administered intravenously by tail vein injection every 4 days for a total of three doses (days 1, 5, and 9). The dosing volume of TTs and irinotecan was determined based upon a ratio of 200 μL for a 20 g mouse and was scaled appropriately according to the actual body weight of the mice. The dosing volume of CPT was determined based upon a ratio of 100 μL for a 20 g mouse and was scaled appropriately according to the actual body weight of the mice. Tumor volumes were measured and recorded every other day. The body weights were measured and recorded every day or every other day. The tumor volume was determined by measuring the tumor in two dimensions with calipers and using the formula “tumor volume=(length×width²)/2”. Each animal was euthanized once the tumor volume reached the predetermined end point size of 2000 mm³.

Biodistribution Study

Mice bearing subcutaneous tumors were established by injecting 100 μL of a U87 MG suspension in serum-free medium (2×10⁶ cells) into the right flank of the mice. When the tumor reached a size of −200 mm³, the mice were randomly grouped and received one of the following 3 treatments via intravenous injection (n=18 for each group): free CPT (4.5 mg/kg), TT 1 (4.5 mg/kg), and TT 1 (15 mg/kg). Three mice from each group were euthanized at pre-determined time points (1, 2, 4, 8, 12, and 24 h). Plasma, major organs, and tumors were collected and stored at −80° C. for further analysis. To determine the amount of free CPT and TT 1 in the samples, 100 mg tumors were mixed with 900 μL acetonitrile (containing 0.1% TFA) and then homogenized using Precellys Evolution Super Homogenizer (Bertin Technologies, France) for 3×40 s (5,600 shakes/min). The resulting suspensions were centrifuged and the supernatants were collected. For plasma, 50 μL of sample was mixed with 450 μL acetonitrile (containing 0.1% TFA) and sonicated for 1 min. The supernatants were collected using centrifugation. All samples were filtered through a 0.22 μm membrane before analysis by a UPLC system (Waters ACQuity™ Ultra Performance LC) equipped with a reverse-phase column (ACQuity UPLC@BEH, C18, 1.7 μm 2.1×150 mm) and a fluorescence detector (ACQuity FLR, Ex/Em=362/430 nm). The column was flushed with a mixture of water (0.1% TFA) and acetonitrile (0.1% TFA) at 0.3 mL/min with the following gradient: 5% acetonitrile (0-1 min), 5-95% acetonitrile (1-5 min), 95% acetonitrile (5-8 min), 95-5% acetonitrile (8-9 min), and 5% acetonitrile (9-10 min). Peaks with retention times of 5.4 min (free CPT) and 6.9 min (TT 1) were monitored.

Antitumor Efficacy Study for Local Delivery

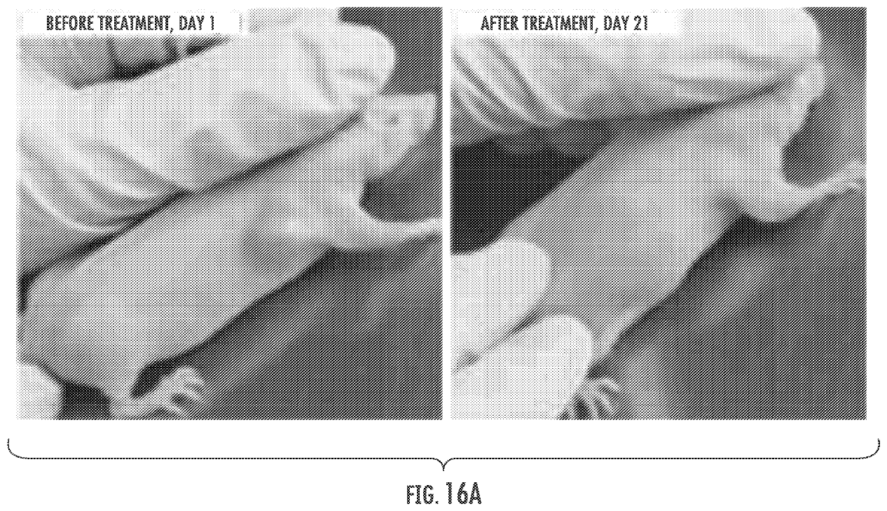

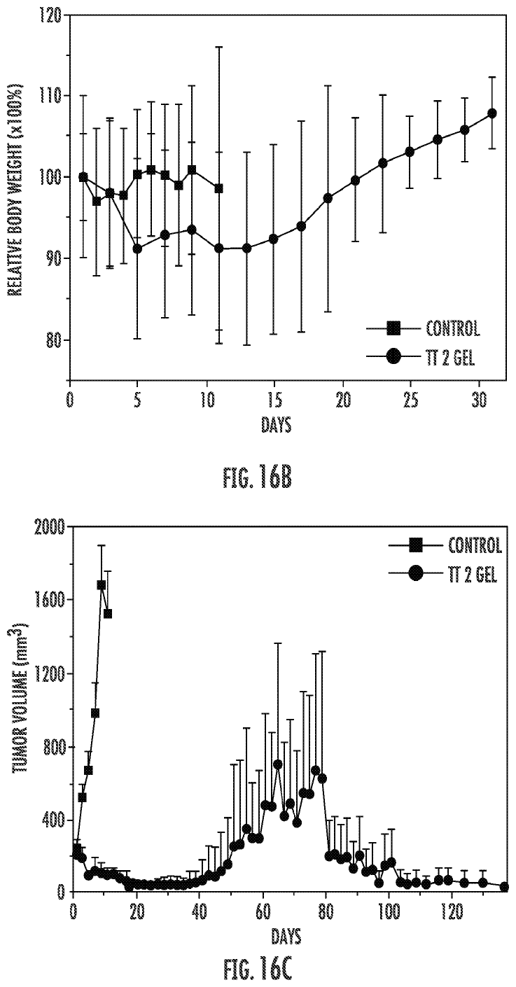

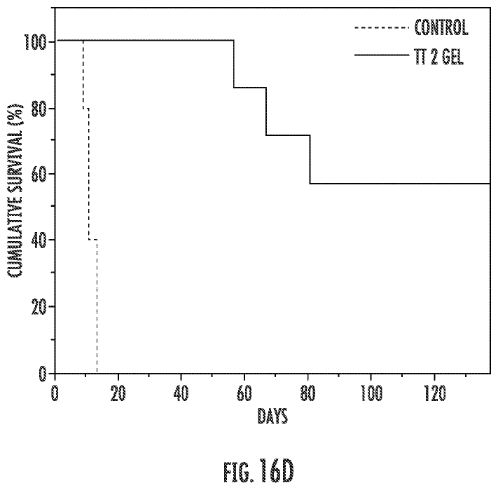

The hydrogel formation of TT 2 in vivo was investigated through subcutaneous injection of a TT 2 gel. The yellowish hydrogel formed immediately after injection and remained there for at least one week (FIG. 16). The tumor model used in local delivery is the same U87 MG human glioblastoma line used in the systemic delivery study described above. Mice were randomly selected (n=7) and treated with TT 2 hydrogel at a fixed dose (10 mM, 30 μL) through intratumoral injection. Tumor volumes and body weights were measured and recorded every other day. The tumor volume was determined by measuring the tumor in two dimensions with calipers and using the formula “tumor volume=(length×width²)/2”. Each animal was euthanized once the tumor weight reached the predetermined end point size of 2000 mm³.

Encapsulation of Functional Dye Molecules

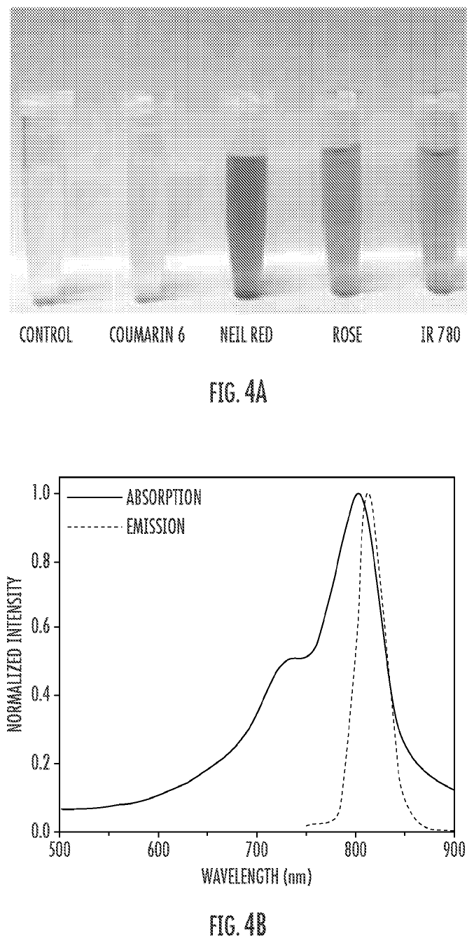

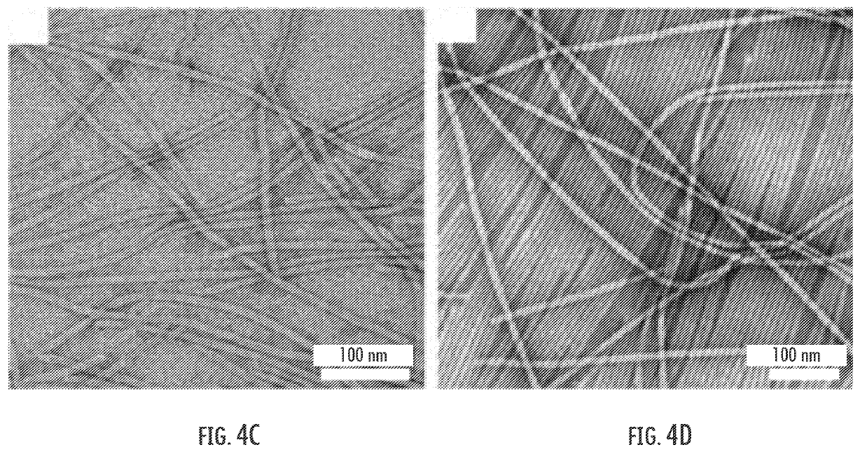

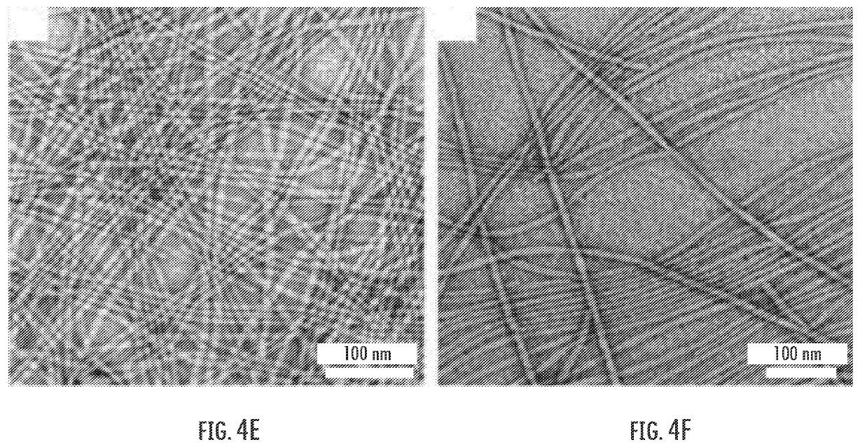

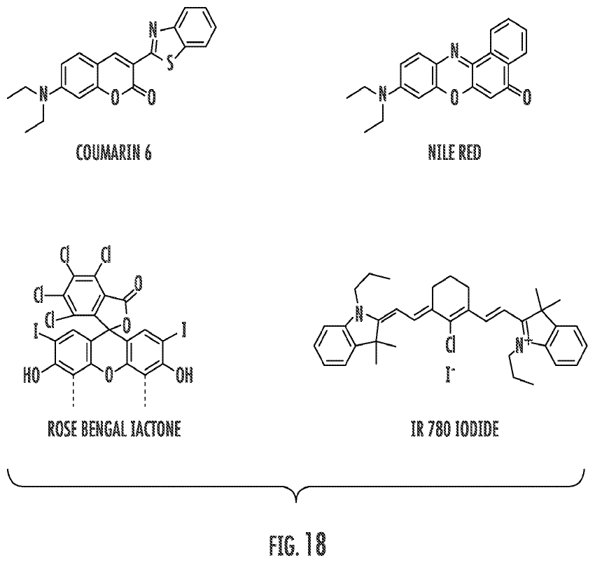

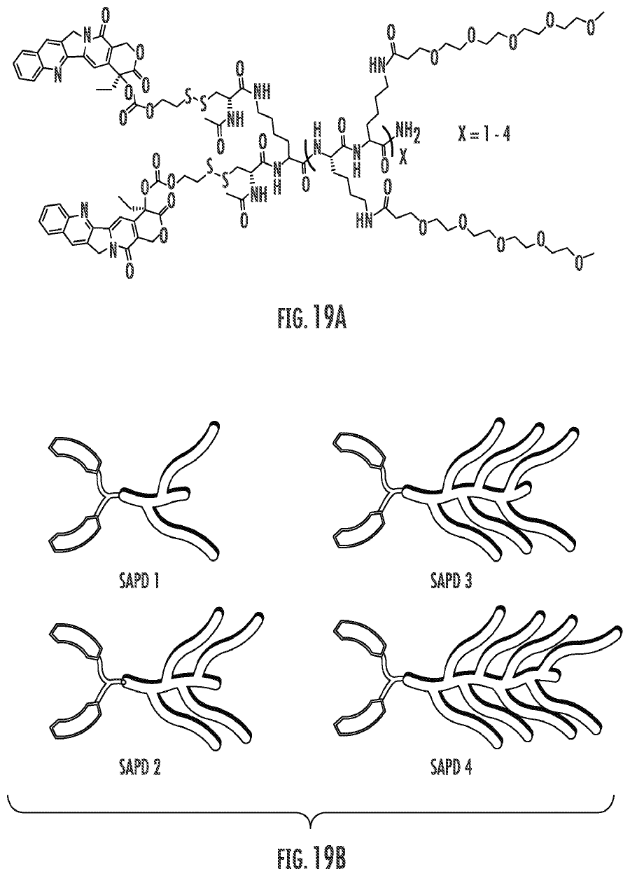

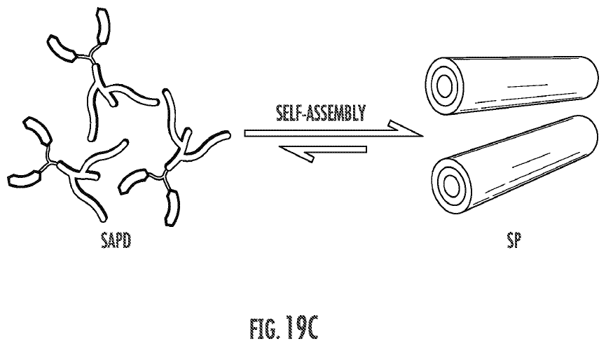

A 300 μM in water stock solution of TT 1 prodrug was prepared and aged overnight to form nanotubes. Hydrophobic dyes, such as Coumarin 6 (C6), Nile Red, Rose Bengal lactone (Rose), and IR 780 iodide (IR 780), were dissolved in acetonitrile at a concentration of 600 μM (FIG. 18). To perform the dye encapsulation experiments, 400 μL of the pre-formed nanotube solution and 200 μL of the corresponding dye solution in acetonitrile were mixed together to make a final solution of 600 μL (H₂O/MeCN=2:1, v/v) in which the concentration of both prodrug and dye was 200 μM (prodrug/dye=1:1, mol/mol). The resulting mixed nanotube and dye solution was aged overnight to allow the penetration of dye molecules into the nanotube structures and then directly lyophilized to fully remove all the solvents. Next, the lyophilized powder was reconstituted to a final prodrug concentration of 200 μM by the addition of 600 μL of water and vortexing for 60 s. The solutions were aged for at least 6 h before being centrifuged (6000 rpm, 5 min) to remove any precipitated free dye. The supernatant was collected for further study. The dye loading capacity was calculated from the percentage of dye in the supernatant to the sum of encapsulated dye and added prodrugs. To get a higher resolution of dye-doped TT 1 solution, the solutions imaged in FIG. 4A were concentrated to a concentration of 800 μM.

Encapsulation of Hydrophobic Drugs

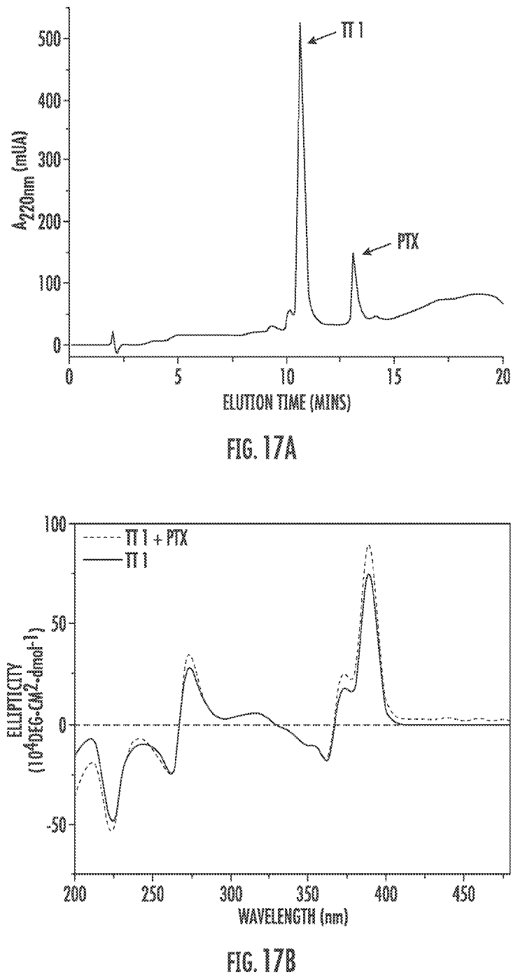

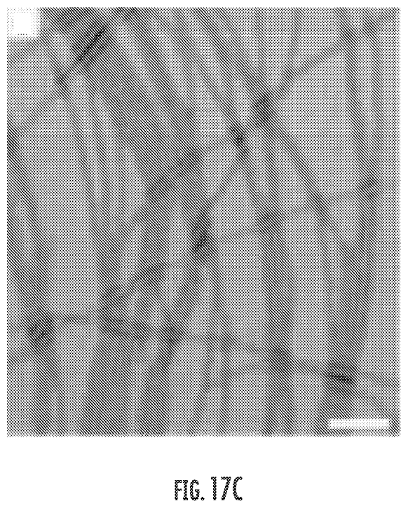

In addition to functional dye molecules, the hydrophobic drug paclitaxel (PTX) was also used as a model compound to dope the TT 1 nanotubes, following the procedure described in the paragraph describing CD measurements of TT solutions above. After encapsulation, analytical HPLC was used to analyze the components of drug-doped nanotubes, monitoring the absorption at 220 nm. As shown in FIG. 17A, a new peak around 13 minutes corresponding to that of PTX is present and indicates successful encapsulation. The concentrations of both nanotube and PTX were determined by comparing the area under the curve of each component with its standard calibration curve, yielding an encapsulation efficiency around 11%. The CD spectrum of PTX-doped nanotube was also recorded, showing a slight decrease in intensity compared with a pure nanotube solution at the same concentration (FIG. 17B). TEM imaging (FIG. 17C) revealed the expected tubular morphology of PTX-doped nanotubes with a diameter of 10.6±0.9 nm. All these results suggest the successful incorporation of PTX drug molecules into the TT nanostructures with no disruption to the tubular morphology.

CD measurement of SAPDs in physiological environments. Stock solutions of SAPDs were prepared at 2 mM and aged overnight. The solutions were diluted to 200 μM in 10% fetal bovine serum (FBS), 10% mice plasma and 10% rat plasma, aged overnight before CD measurement. To study the kinetic stability of the assembled SAPDs upon dilution, the stock solutions of SAPD 1 and 2 were diluted to 100 μM, 50 μM, 25 μM, 10 μM, and 5 μM in 10% rat plasma. The CD spectra were recorded at 5 min, 1 h, 4 h, and 12 h. All the CD spectra were recorded from 300 to 450 nm using a 1 mm path length quartz cell. The spectra were collected and normalized from ellipticity (mdeg) to molar ellipticity (deg·cm2·dmol⁻¹).

Drug release and chemical stability studies of SAPDs. Drug release studies of four SAPDs were performed at a concentration of 200 μM in PBS buffer with or without the reducing agent glutathione (GSH). Briefly, 400 μM stock solutions of SAPDs in water were prepared and aged overnight. Stock solutions containing 2×PBS (20 mM) with or without 20 mM GSH were prepared 1 h before the experiment, and the pH was tuned to 7.4 with NaOH. The prodrug solutions were further diluted to 200 μM with 20 mM (2×) PBS buffer with or without GSH (20 mM) to give final solutions of 200 μM prodrug, 10 mM PBS and with or without 10 mM GSH. Three replicates of each SAPD were prepared with or without GSH and were incubated at 37° C. Samples with GSH were collected at 0 min, 5 min, 10 min, 15 min, 30 min, 1 h and 2 h, while samples without GSH were collected at 0 h, 6 h, 12 h, 24 h, 48 h, 72 h, 96 h and 120 h. To prevent further reaction after sample collection, the collected samples (50 μL each point) were acidified by adding 0.2 μL of 2M HCl, frozen with liquid nitrogen and stored at −30° C. The release profile was determined by analytical RP-HPLC using the following conditions: Varian Pursuit XRs C18 (5 μm, 150×4.6 mm); 362 nm detection wavelength; 1 mL/min flow rate; the gradient began at 15% to 85% acetonitrile containing 0.1% TFA by 15 min and back to initial gradient at 18 min. The calculated data points were plotted as a percentage of the total CPT concentration against time. Representative HPLC traces over time were also integrated for comparison.

The drug release in rat plasma (10%, v/v) were performed using similar protocols as those in PBS. To determine the amount of SAPDs and free CPT in each sample, 50 μL of sample was mixed with 200 μL acetonitrile (containing 0.1% TFA) and sonicated for 1 min. The supernatants were collected using centrifugation. All samples were filtered through a 0.22 μm membrane before analysis by a UPLC system (Waters ACQuity™ Ultra Performance LC) equipped with a reverse-phase column (ACQuity UPLC@BEH, C18, 1.7 μm 2.1×150 mm) and a fluorescence detector (ACQuity FLR, Ex/Em=362/430 nm). The column was flushed with a mixture of water (0.1% TFA) and acetonitrile (0.1% TFA) at 0.3 mL/min with the following gradient: 5% acetonitrile (0-1 min), 5-95% acetonitrile (1-5 min), 95% acetonitrile (5-8 min), 95-5% acetonitrile (8-9 min), and 5% acetonitrile (9-10 min). Peaks of SAPDs and free CPT were monitored and recorded, and the concentrations were calculated by comparing with standard curves.

Antitumor efficacy study of SAPDs at the same dose on a HT-29 tumor model. HT-29 tumor model was established by subcutaneously (s.c.) injection of 5×10⁶ HT-29 cells into the right shoulder of athymic nude mice (8-9 weeks old). When the averaged tumor size reached 75-95 mm³, mice were randomly divided into six groups. Four different SAPDs were all intravenously (i.v.) dosed at 10 mg/kg (CPT equivalent) at days 1, 5, 9 and 13 (n=6 for each group) with PBS (n=5), free CPT (n=5, intraperitoneal (i.p.) injection, 9 mg/kg at days 1, 5, 9 and 13) and irinotecan (n=5, i.p. injection, 100 mg/kg at days 1, 8 and 15) as controls. Hydrophobic CPT was dissolved in a mixture of DMSO/ethanol/PEG-400/water at a volumetric ratio of 1:1:2:1, and administrated through i.p. injection with a total volume of 100 μL to minimize the toxicity of organic solvent. Based on our experience, i.v. injection of 100 μL organic solvent will result in immediate death of the studied mice as a result of solvent-realted toxicity, but i.p. injection of 100 μL solvent is tolerable. Here, the dose of irinotecan is its MTD as reported in literatures, and i.p. injection achieved similar efficacy to i.v. injection while reducing the side effects. The dosing volumes of SAPDs and irinotecan were estimated by a ratio of 200 μL for a 20 g mouse. The tumor volumes and body weights were monitored and recorded every two or three days. The tumor volume was estimated by measuring the length and width with calipers and using the equation “tumor volume=(length×width²)/2”. Mice were euthanized once the tumor volume reached 1000 mm³.

Circulation studies of SAPDs in rats. Female Sprague Dawley (SD) Rats (200-250 g) were randomly grouped into four groups with three rats in each group. SAPDs were all intravenously (i.v.) dosed at 10 mg/kg (CPT equivalent). The dosing volumes of SAPDs were estimated by a ratio of 1 mL for a 200 g rat. The blood samples were collected at 5 min, 15 min, 30 min, 1 h, 2 h, 4 h, 8 h, and 12 h, which were immediately centrifuged to take plasma for further analysis and stored at −80° C. The plasma proteins were precipitated using similar protocols as mentioned above in drug release section and the determination of SAPDs and free CPT in the plasma by UPLC also used the same conditions.

Estimation of SAPD concentration in plasma upon injection. To calculation the concentration after dilution, we simply set the following parameters: body weight of mice (20 g), dosing volume of mice (200 μL), blood volume of mice (1.8 mL), body weight of rat (200 g), dosing volume of mice (1 mL), blood volume of mice (18 mL) and the dosage for both mice and rats (10 mg/kg).

The equation is:

$$\frac{dosage \frac{mg}{kg} ( C P T equivalent ) \times body weight ( kg )}{\begin{matrix}Mw of C P T \times 2 ( two C P T on each prodrug ) \times \\ ( blood volume + injected volume ) mL\end{matrix}} = 144 µM$$

$$In the case of mice , the concentration upon dilution will \text{be:}$$

$$\frac{10 \frac{mg}{kg} \times 0.02 kg}{\begin{matrix}34 \; 8.352 \frac{g}{mol} ( Mw of C P T ) \times 2 \\ ( two C P T on each prodrug ) \times ( 1.8 + 0.2 ) mL\end{matrix}} = 144 µM$$

$$In the case of rats , the concentration upon dilution will \text{be:}$$

$$\frac{10 \frac{mg}{kg} \times 0.2 kg}{\begin{matrix}34 \; 8.352 \frac{g}{mol} ( Mw of C P T ) \times 2 \\ ( two C P T on each prodrug ) \times ( 18 + 1 ) mL\end{matrix}} = 151 µM$$

Maximum tolerated dose (MTD) studies. MTD of SAPD 1 has been previously determined in our lab (Table 1). MTDs of other SAPDs were determined by dose escalation studies in healthy female athymic nude mice (Charles River, 12-13 weeks old). A single intravenous (i.v.) injection of SAPD was dosed at day 1 and the body weights of each mouse (n=3) were recorded every day for six days and every the other day until two weeks. The dosing volume was determined based upon a ratio of 200 μL for a 20 g mouse. Doses of SAPDs used in the studied were 108, 72, 54, 45, 36 and 18 mg/kg (CPT equivalent). The maximum tolerated dose (MTD) was determined by the largest dose that did not result in more than a 20% body weight loss or death of an animal (Table 2).

Antitumor efficacy study of SAPDs at near MTD on a HT-29 tumor model. HT-29 tumor model was established as described above. When the averaged tumor size reached 70-110 mm³, mice were randomly divided into six groups (n=5 for each group). Four different SAPDs were all i.v. dosed at near or slightly lower than their estimated MTDs. According to our experience, the MTD of multiple injections (three doses and four days a dose) could be around ½ of the MTD of a single injection (see Tables 1 and 2). For example, MTD of SAPD 1 of multiple injections is 12 mg/kg, which is ½ of the MTD (24 mg/kg) of a single injection. The estimation is consistent with our previous findings. Thus, in this study, the dose of SAPD 1 is 12 mg/kg (½ MTD). The dose of SAPD 3 and 4 is 36 mg/kg (½ MTD). Although SAPD 2 has slightly lower MTD, we also used the same dose as SAPD 3 and 4 to make them consistent. The irinotecan (i.p. injection, 100 mg/kg at days 1, 8 and 15) and the PBS groups were used as controls. All other protocols were similar to those in the above-mentioned efficacy study.

### Example 1

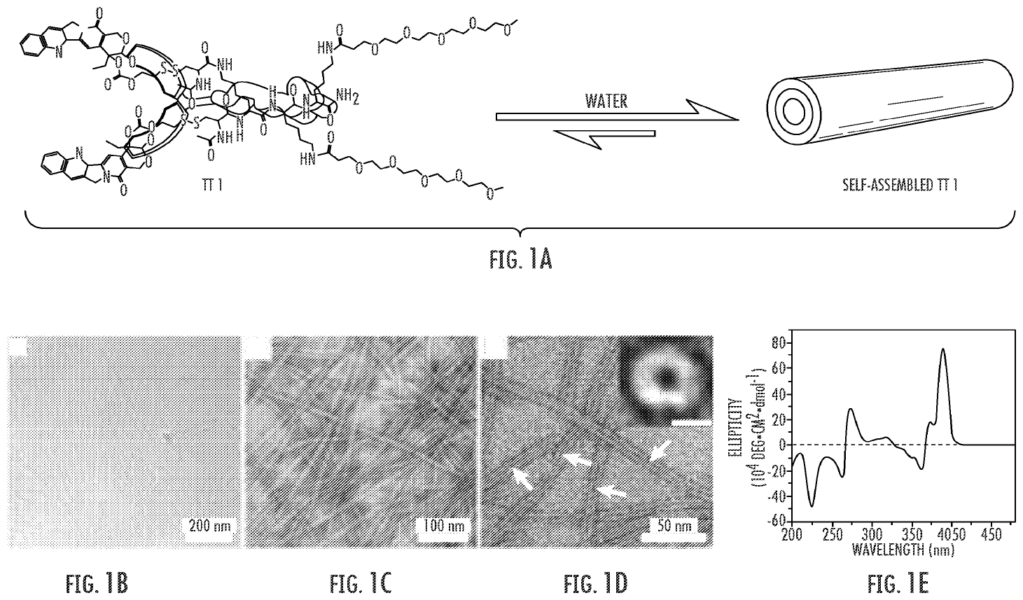

FIG. 1A displays the chemical structure of TT 1, comprising a short oligoethylene-glycol (OEG) segment and two CPT moieties (FIG. 5, and FIG. 6). A representative cryogenic transmission electron microscopy (cryo-TEM) image is shown in FIG. 1B, revealing filamentous assemblies of TT 1 in water. Conventional TEM imaging with negative staining corroborates the cryo-TEM observation (FIG. 1C), measuring ˜8.8 nm in width (Table 1) and several micrometers in length, and further discloses a dark centerline (marked with white arrows in FIG. 1D). This emblematic dark centerline is indicative of the hollowed interior of the observed filaments, resulting from preferential deposition of the negative staining agent on the collapsed tubular structures. The tubular nature of the TT 1 assemblies can be further confirmed by the occasional observations of toroidal structures (FIG. 1D inset and FIG. 7). The wall thickness measured from these toroids is 3.0±0.5 nm, with a hollow interior diameter of 2.5±0.6 nm, which suggests a monolayered rather than bilayered packing, reminiscent of tubular macrocycle assemblies (16, 17). Circular dichroism (CD) spectroscopy of TT 1 at 200 μM suggests that the aromatic CPT units are arranged in a highly ordered fashion (FIG. 1E). The two bisignate CD signals centered at 266 and 367 nm are a result of strong exciton coupling among neighboring CPT aromatic rings, with their positive nature implying a right-handed helical arrangement (18). The negative signal around 223 nm arises from intermolecular hydrogen bonding among the peptide segments, in a manner similar to the peptide arrangement observed in typical β-sheet assemblies.

Table 1. Diameters of self-assembled TT nanotubes measured from conventional-TEM (n>40). The lengths of these nanotubes are all on the micrometer scale and the diameters are 8.8 nm for TT 1 and in the range of 8.1-8.4 nm for TT 2-4, as measured from TEM images. The diameters of TT 2-4 are slightly smaller (˜0.5 nm) than TT 1 because OEGs on the lysine side chain extend the molecular length of TT 1. The measured diameters strongly support the monolayered packing model for these tubular aggregates.

| Self-assembled Tubustecans | Diameters measured by conventional TEM |
|---|---|
| TT 1 | 8.8 ± 0.8 nm |
| TT 2 | 8.1 ± 0.6 nm |
| TT 3 | 8.4 ± 0.9 nm |
| TT 4 | 8.3 ± 0.9 nm |
| TT 5 | 8.4 ± 0.9 nm |

### Example 2

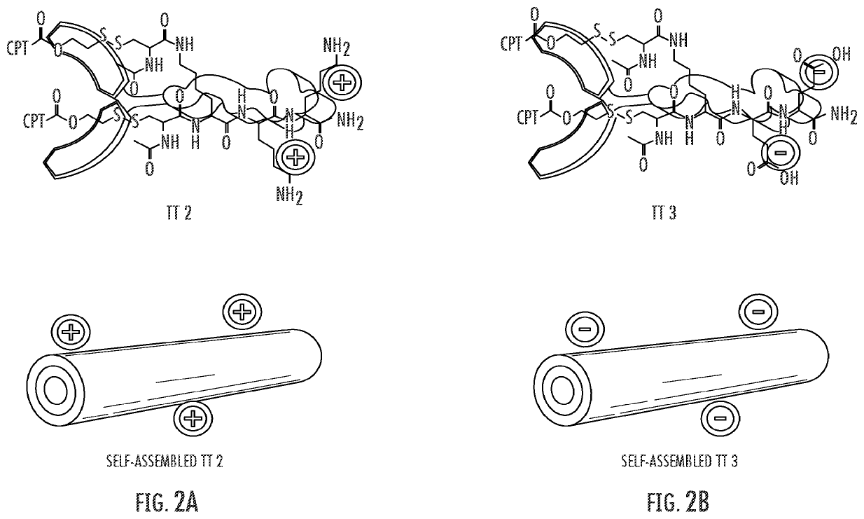

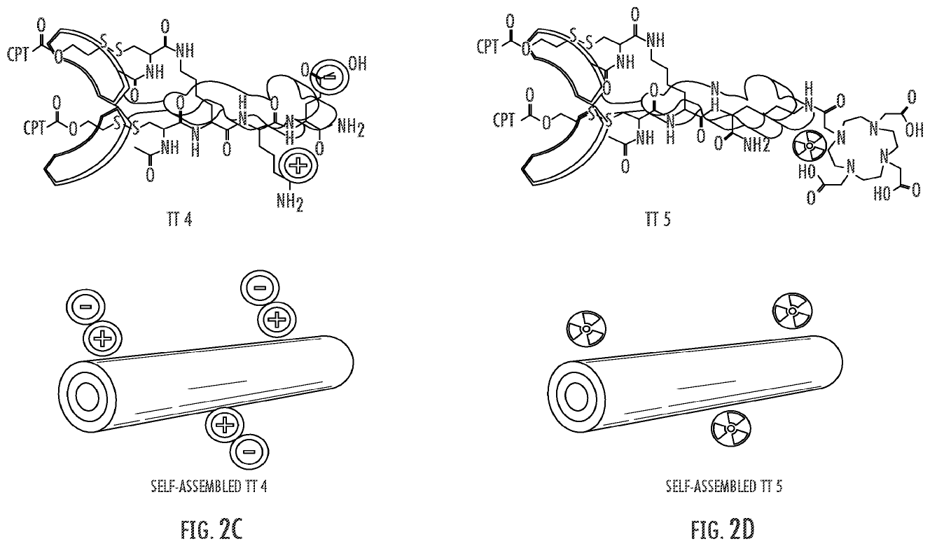

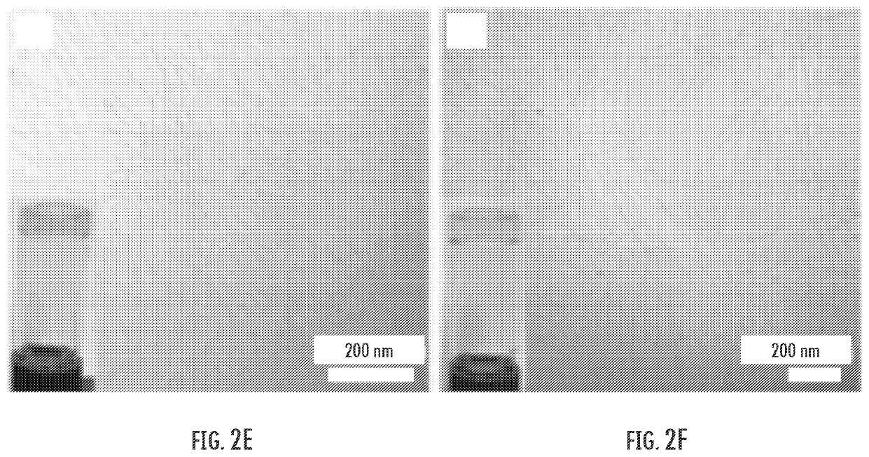

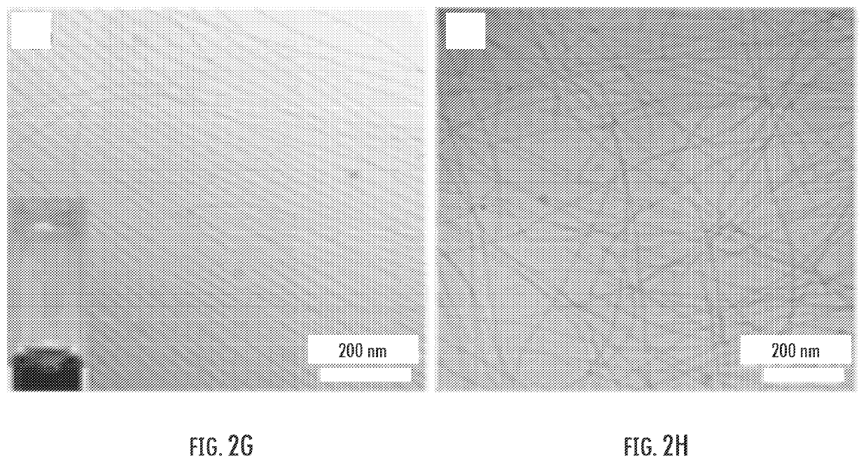

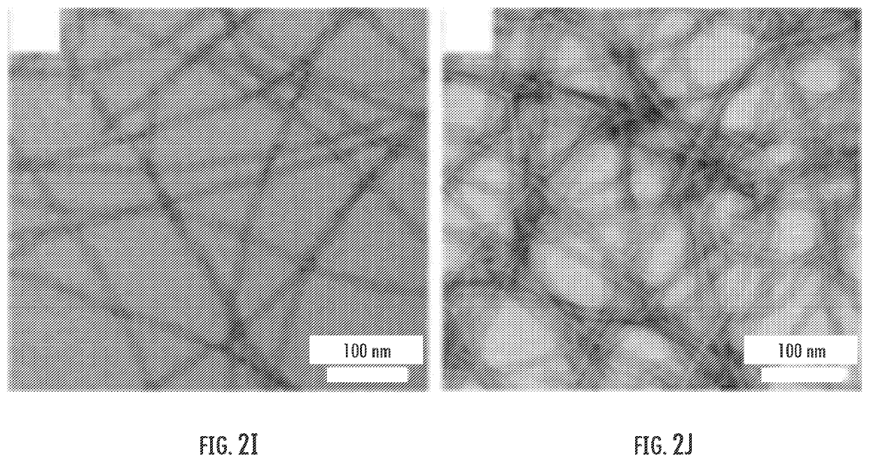

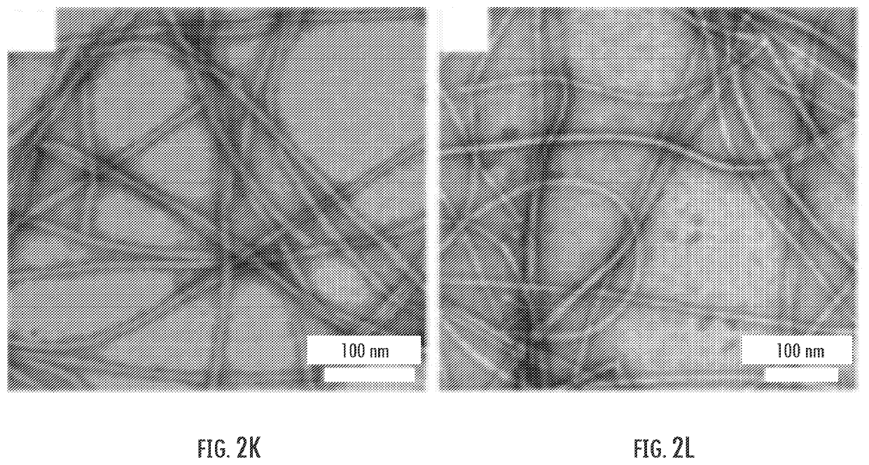

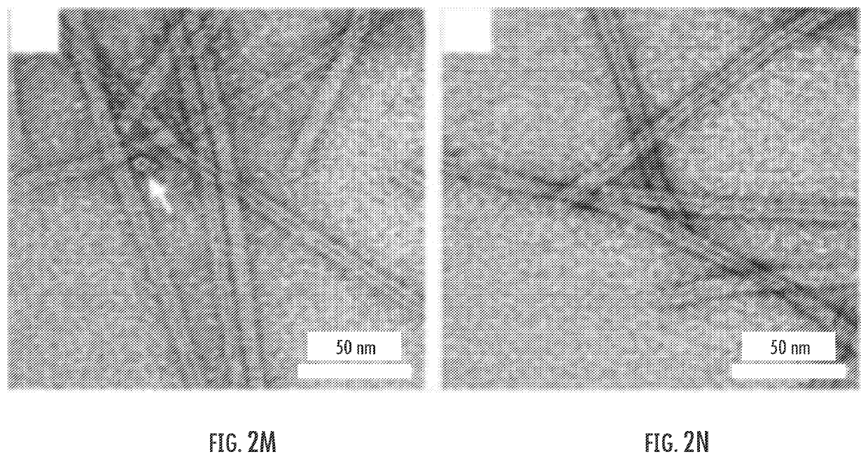

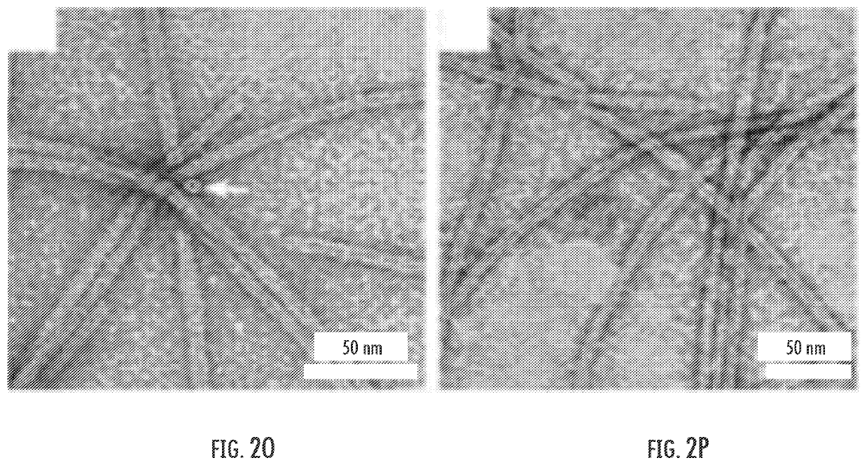

We found that the tubular assembly protocol is remarkably tolerant to the choice of hydrophilic segment. FIGS. 2A-D shows an additional four Tubustecan designs, including the cationic TT 2, the anionic TT 3, the zwitterionic TT 4, and the metal-chelating TT 5 with DOTA as the hydrophilic segment. Cryo-TEM (FIG. 2E-H) and conventional TEM imaging (FIG. 2I-P) confirms the tubular assembly for each TT design, all with a length on the micrometer scale and a diameter of 8.5 to 8.9 nm (Table 1). We recorded their respective CD spectra at 200 μM (FIG. 8A), clearly revealing that all TTs 2-5 exhibit the characteristic two bisignate CD signals at 266 and 367 nm, accompanied with a strong positive signal at 389 nm. After normalization, the CD spectra are nearly indistinguishable (FIG. 8B), suggesting a high level of similarity among the various assemblies and validating the robustness of the Tubustecan design protocol. The independence of tubular formation on the dramatically varied hydrophilic chemistry strongly suggests that it is the associative interactions among CPT units that play a predominant role in defining the tubular morphology. Indeed, the critical micellization concentrations (CMCs) for all TTs, as measured by the Nile Red method (19), fall within the range of 2-5 μM despite their distinction in hydrophilicity (FIG. 9). As a result of their charged and pH-responsive features (FIGS. 10A and 10C), TTs 2-4 nanotubes form self-supporting hydrogels in a PBS buffer at concentrations of 5 mM or higher (FIGS. 2E-H insets). The inclusion of DOTA in TT 5 expands the functionality of tubular SPs to radiopharmaceutical imaging or magnetic resonance imaging through chelation with contrast agents (20).

### Example 3

Given that the biological functionality of these supramolecular polymers is only associated with the free CPT form in the monomeric state, we assessed the in vitro release behavior of the non-ionic TT 1 nanotubes, a potential candidate for systemic delivery (21), and that of the TT 2 hydrogel which can potentially serve as a depot for local treatment. FIG. 3A clearly demonstrates that the tubular assemblies of TT 1 can be effectively converted to the bioactive form in the presence of the reducing agent glutathione (GSH), with 80% of free CPT molecules released within 2 h (FIG. 11). We also assessed the short-term stability of TT 1 SPs in phenol red-free cell medium with 10% FBS using CD spectroscopy (FIG. 12) and their long-term stability in PBS using Zeta Potential measurements (FIGS. 10B and 10D), both showing minimum dissociation or aggregation at concentrations greater than 25 μM. Importantly, the TT 2 hydrogel exhibited a long-term and near-linear release profile, with ˜10% of TT 2 liberated from the hydrogel over a one-month period (FIG. 3B). The linear and concentration-independent release can be attributed to the unique feature of supramolecular systems, which maintains a constant monomer concentration above the CMC value (22). As a result of their effective release and conversion, both TT 1 and TT 2 exhibited a high potency against U87 MG human brain cancer cells, with their respective IC₅₀ values of 149 nm and 123 nM (IC₅₀ is the half maximal inhibitory concentration that kills 50% population of cells tested). Since these IC₅₀ values are much lower than their CMCs, we speculate that it is the monomeric forms of TT 1 and TT 2, not their supramolecular assemblies that exerted the biological function against cancer cells. These results also reveal that the pharmaceutical activity of unassembled CPT analogues are comparable to that of free CPT (IC₅₀: 62 nM) but far superior to that of irinotecan (IC₅₀: 6505 nM), the CPT prodrug currently used in clinical treatments. In the case of irinotecan, the prodrug must be metabolized to fully restore its activity (23).

### Example 4

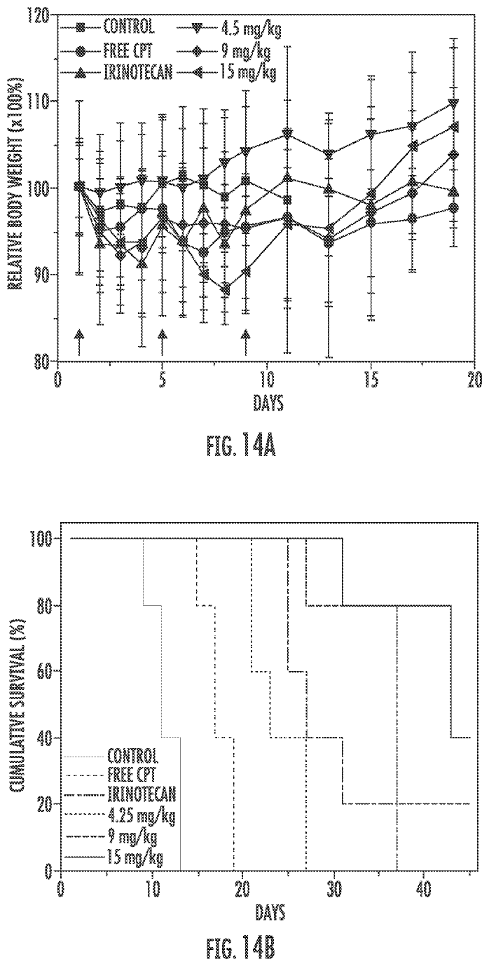

We found that the self-assembly of TT 1 into tubular SPs significantly improves both the maximum tolerated dose (MTD) for rodents and their systemic treatment outcomes. Due to its poor water solubility, free CPT is often given in a formulation containing a mixture of DMSO/ethanol/PEG-400/water at a volumetric ratio of 1:1:2:1 (24). At a dosage of 9 mg/kg, intravenous administration resulted in immediate death of the studied mice. We eventually identified an intraperitoneal injection of 4.5 mg/kg CPT to be a tolerable dosage for animal studies. In contrast, the MTD of TT 1 SPs in healthy athymic nude mice, identified in a dose escalation study through systemic administration, is within the range of 24-30 mg/kg (CPT equivalent) (FIG. 3D and FIG. 13). On the basis of these results, we assessed the in vivo antitumor effect of TT 1 through intravenous injection of three different doses (4.5 mg/kg, 9 mg/kg, and 15 mg/kg of CPT equivalent) on days 1, 5 and 9, with non-treatment, free CPT (4.5 mg/kg), and irinotecan as control groups (FIG. 3E and FIG. 14). Free CPT was shown the least effective in suppressing tumor growth, merely improving the median survival from 11 to 17 days (FIG. 3F). At a dose of 4.5 mg/kg, TT 1 can suppress the tumor volume (417 mm³) in the treated mice up to 15 days, comparable to those treated with 60 mg/kg irinotecan. In both cases, the tumor was observed to grow rapidly after the treatment was halted, giving rise to a median survival of 23 and 27 days, respectively. When the TT 1 dose was increased to 9 mg/kg and to 15 mg/kg, we observed that mice in these two groups showed a significant delay in tumor growth, and the mean tumor volumes at three weeks after treatment were 94 mm³ for 9 mg/kg, and 59 mm³ for 15 mg/kg. Four out of five mice survived for up to 37 d (9 mg/kg) and 43 d (15 mg/kg). Given the comparable potency of monomeric TT 1 to free CPT, these results clearly suggest it is the self-assembly into tubular SPs that enables the administration of a much larger dose, greater tumor regression, and prolonged survival.

### Example 5

We next sought to investigate how the formation of TT 1 tubular SPs could alter the circulation properties of the monomeric CPT and restore its pharmaceutical activity in vivo. In a tumor-bearing mouse model, we administered intravenously the same dose of TT 1 and free CPT (4.5 mg/kg) and compared their concentrations in blood and in tumor sites. To facilitate systemic administration, free CPT was formulated using a solvent mixture of DMSO/ethanol/PEG-400/water. As shown in FIGS. 3G and 3H, the SPs significantly increased the drug concentration in plasma by approximately 129-fold (4760 vs. 37 ng/g) and in tumor by 8-fold (731 vs. 92 ng/g) in comparison with free CPT at 1 hour after injection. In contrast to the rapid clearance of CPT from plasma, TT 1 is retained in the blood for up to 12 hours. At a higher dose (15 mg/kg), TT 1 SPs showed even higher circulation concentrations in blood and greater tumor accumulation. We also measured the concentration of free CPT versus the conjugated CPT for TT 1 in the tumor site and found that at 1 hour after injection, 47% and 58% of TT 1 were converted to free CPT in the tumor for injection doses of 4.5 mg/kg and 15 mg/kg groups (FIGS. 3H and 3I), respectively. At 8 hours, the conversion ratios increased to 82% for 4.5 mg/kg and 96% for 15 mg/kg. These studies led us to conclude that the longer circulation time of TT 1 SPs and their effective conversion to free CPT in tumor sites largely contribute to the observed treatment outcomes in our rodent models.

### Example 6

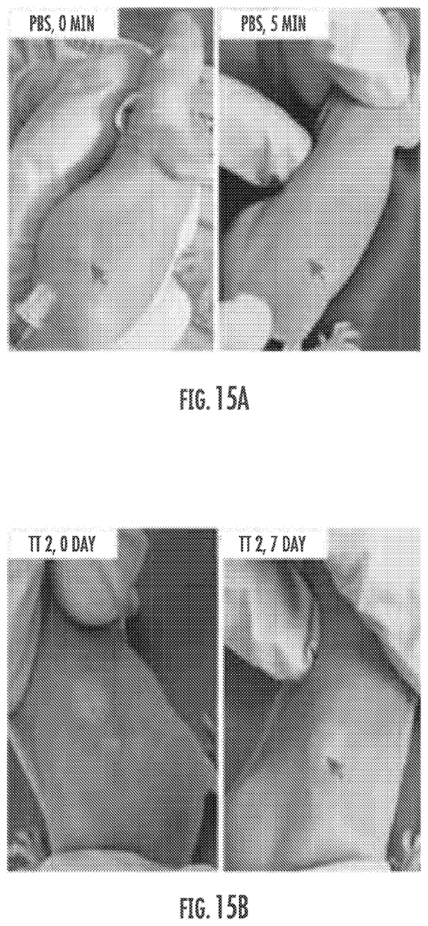

In an effort to evaluate the potential for local delivery, we subcutaneously injected TT 2 SP solutions into athymic nude mice and found that TT 2 formed a yellowish hydrogel immediately after injection and remained in place for at least seven days (FIG. 3J and FIG. 15). In the control experiment, the injected PBS bolus was observed to disappear completely within five minutes after injection (FIG. 15). The antitumor efficacy of TT 2 hydrogel was then evaluated in a subcutaneous U87 MG xenograft model via intratumoral injection of a 10 mM TT 2 SP hydrogel (30 μL). All seven mice studied experienced tumor regression with a minimum mean tumor volume of 36 mm³ at day 25 and survived for more than 45 days (FIG. 3K). Four out of seven mice exhibited complete tumor regression (FIG. 16), suggesting that the TT 2 hydrogel can be sustainably converted into free CPT in tumor sites for a long-lasting antitumor effect.

### Example 7

Having demonstrated the utility of the self-assembling TT platform for both systemic and local treatment, we posited that their inherent functionality could be further complemented by taking advantage of their unique hollow nature. Being bounded by CPT moieties, the tubular cavities that these TT 1 SPs possess is of largely hydrophobic character, implying a potential utility as carriers of other agents. We subsequently found that TT 1 can indeed serve as a universal dispersing agent for a variety of small molecule hydrophobes. After incubation with the TT 1 SPs in a 2:1 mixture of water/acetonitrile (ACN), Coumarin 6 (C6), Nile Red, Rose Bengal lactone (Rose), and IR-780 iodide (IR-780) can spontaneously partition into the tubular assemblies through passive diffusion (FIG. 18). After removal of any unencapsulated dyes and ACN, the resulting colored aqueous solutions (FIG. 4A) fully demonstrate that water-insoluble dye molecules can be effectively dispersed within the TT 1 assemblies, with dye loading contents of 4.5%, 4.4%, 14.8% and 10.3% for C6, Nile Red, Rose, and IR-780, respectively. Notably, the representative IR 780-containing TT 1 solution exhibited a red-shifted absorption maximum of 802 nm and a fluorescence emission centered at 812 nm, indicating its potential suitability for in vivo diagnostic applications (FIG. 4B). Paclitaxel, a hydrophobic drug, can also be successfully dispersed by TT 1 assemblies, yielding an encapsulation efficiency of −11% (FIG. 17). TEM imaging confirmed that the dye/drug encapsulated nanostructures retained their tubular morphology with only a small increase in diameter (around 1-2 nm), betraying any adjustments to accommodate the guest molecules (FIGS. 4C-F and FIG. 17).

### Example 8

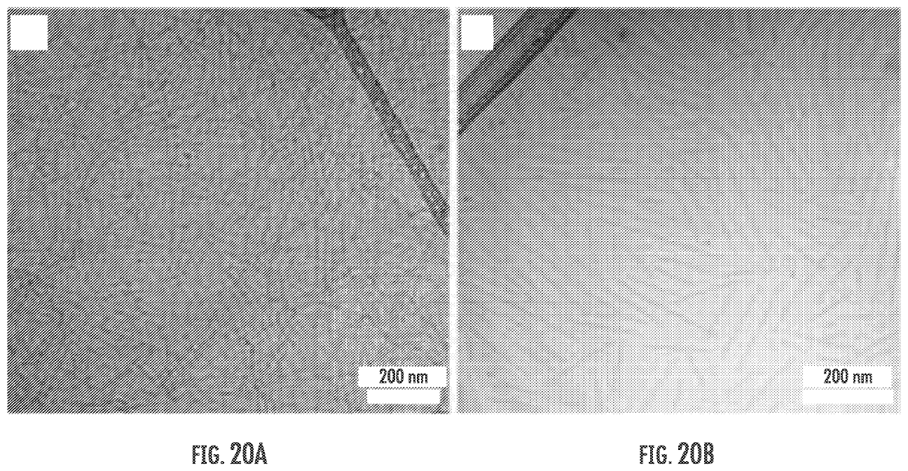

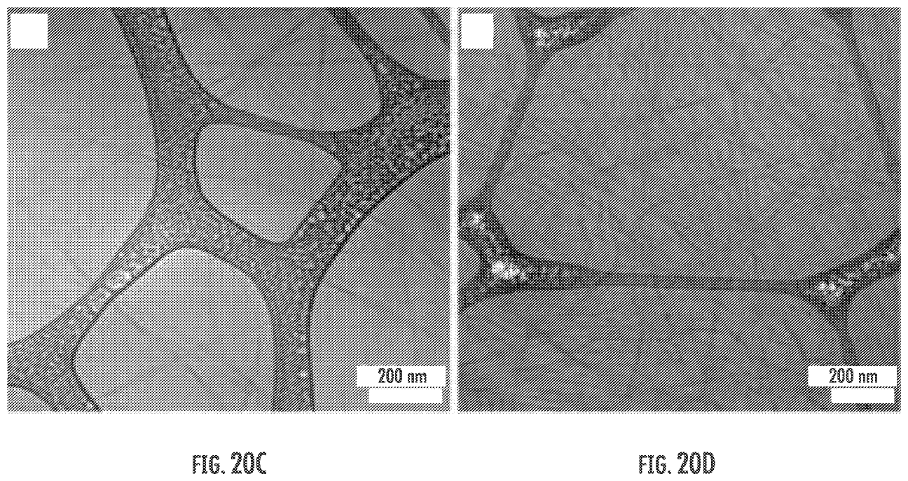

Self-Assembly of SAPDs from TT1. To study the self-assembly behavior of the SAPDs, we directly dissolved the lyophilized powders in deionized water at a concentration of 2 mM and neutral pH. After aging overnight, cryogenic transmission electron microscopy (cryo-TEM) imaging (FIGS. 20A-D) reveals that all the SAPDs can self-assemble into one-dimensional (1D) nanostructures. More specifically, SAPD 1 formed supramolecular filaments of around 9 nm in diameter and several micrometers in length (FIG. 20A), and SAPD 2 self-assembled into shorter filaments with a majority less than 400 nm in length (FIG. 20B). Conventional TEM images (data not shown) clearly reveal the hollowed filaments of assembled TT1 (2OEG) and 2, a result of highly ordered packing of CPT moieties within the hydrophobic core. SAPD 3 (FIG. 20C) and 4 (FIG. 20D) both aggregated into micrometer-long nanoribbons of various widths with slight twisting observed. The formation of those 1D assemblies is believed to be a result of strong π-π interactions among CPT units, acting in concert with the intermolecular hydrogen bonding among the OEGlayted peptides. The complex interplay of these two associative interactions promotes the directional growth of the observed SPs and defines the resulting morphology, despite no inclusion of any β-sheeting-forming peptide sequences in the molecular design. The slight differences in the length and morphologies could be plausibly attributed to the increase of overall HLB values and steric hindrance caused by OEG moieties that weaken the hydrophobic associations, and also the strengthened intermolecular hydrogen bonding capacity that could shift the directional associations from π-π dominant mode to hydrogen bonding controlled manner. Furthermore, the neutral surface chemistries of the SPs were confirmed by ζ-potential measurement of all four assembled SAPDs in the PBS buffer, showing slightly negative values of −6.5 mV, −7.4 mV, −6.5 mV, and −5.9 mV for SAPD 1-4, respectively (data not shown). These results indicate that all SAPDs can assemble into SPs, albeit with slight variations in length and morphology, that are solely made of prodrugs without any external materials or pharmaceutical excipients.

### Example 9

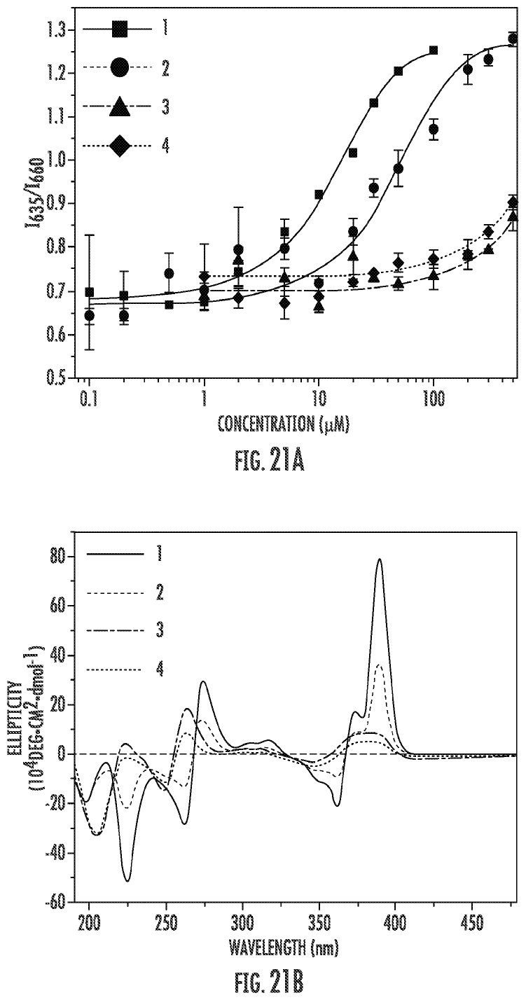

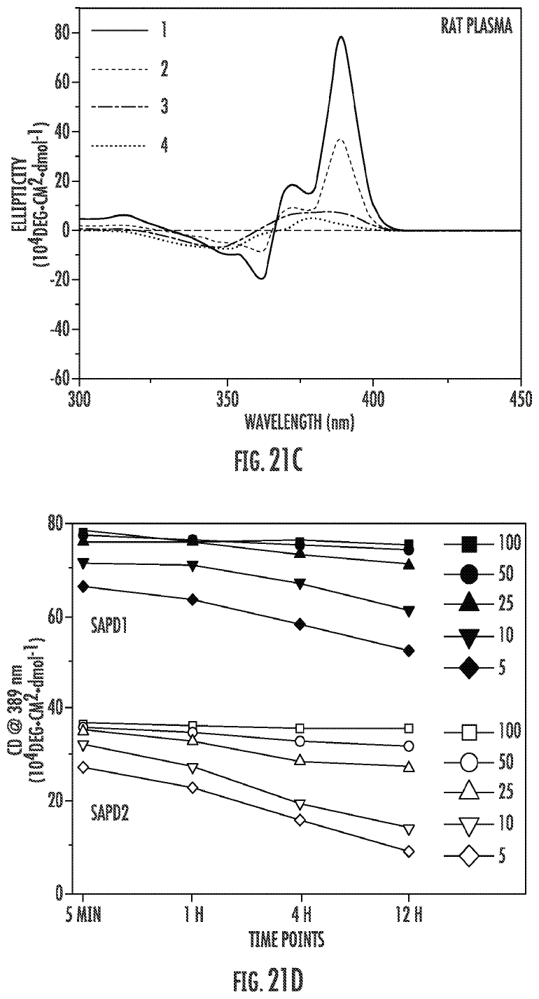

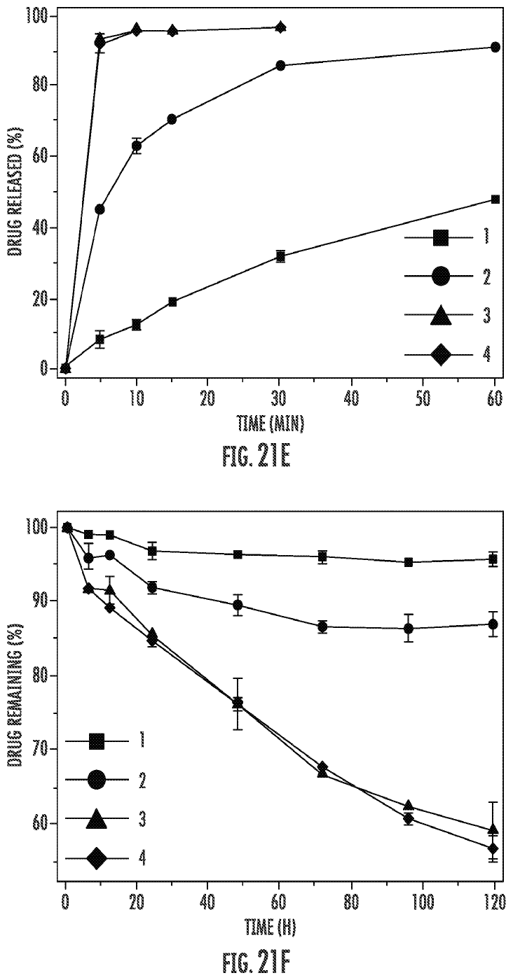

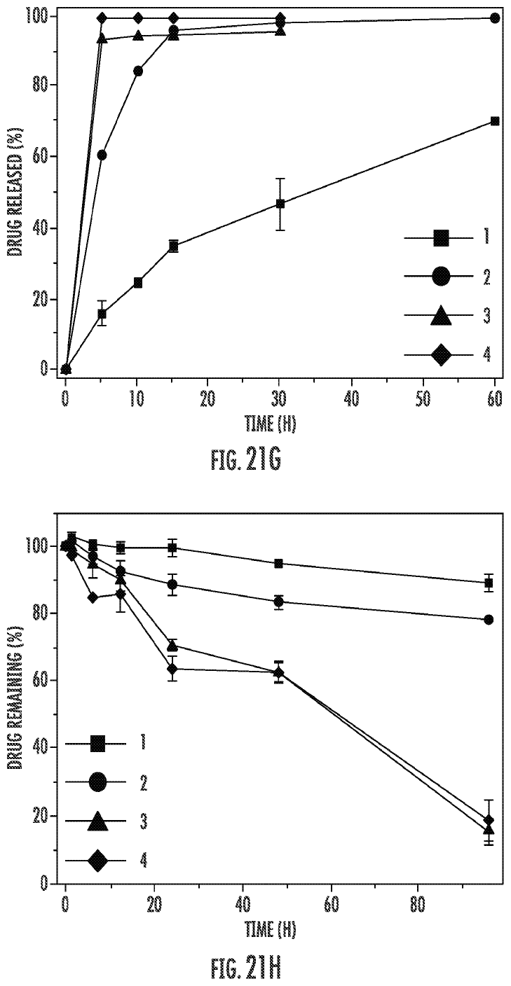

CMC Measurement of SAPDs of TT1. We next assessed the CMC values for each of the designed SAPDs in aqueous solution. It is worth mentioning that for peptide-based amphiphiles it is not uncommon that the CMC values differ from their critical assembly concentration (CAC). Velichko et al. revealed in a simulation study that peptide amphiphiles with a strong hydrogen bonding sequences could first assemble into β-sheets before micellization into well-defined supramolecular nanofibers with a hydrophobic compartment. Experimentally, the CAC is often measured using spectrometry techniques such as circular dichroism (CD) to reveal the presence of intermolecular associations and packing at a much lower concentration. In the case reported here, the CMC values were measured in the PBS buffer using Nile Red as a probe, which fluoresces intensely in hydrophobic environments and is strongly quenched and red-shifted in aqueous media. Measuring fluorescent spectra excited at 550 nm of SAPD solutions of varying concentrations, and then plotting the ratio of intensity at 635 nm (emission maximum of the Nile Red in hydrophobic environment) to that at 660 nm (emission maximum in aqueous conditions) against the concentration of SAPDs yielded the plots shown in FIG. 21A. According to the changes in fluorescence intensity, the CMC values are estimated to be 2.7 μM and 10.1 μM for SAPD 1 and 2, respectively. The CMCs of SAPD 3 and 4 exceed 200 μM and cannot be accurately extracted. The CMC experiments suggest that structural stability of the SAPD SPs would be SAPD1 (OEG2)>SAPD 2 (OEG4)>SAPD 3 (OEG6) and SAPD 4 (OEG8) and that SAPD 3 and SAPD 4 are unable to form stable assemblies at the concentration of sub-mM range.

### Example 10

Molecular Packing of SAPD Monomeric Units in the SPs. To further validate our hypothesis that the increase of OEG repeat numbers in peptides would decrease the supramolecular stability of the resulting SPs, we performed circular dichroism (CD) spectroscopy measurements to understand the molecular arrangement within the SPs at the concentration of 200 μM (FIG. 21B). CD spectrum of assembled SAPD 1 shows two bisignate signals centered at 266 and 367 nm, and a strong positive signal at 389 nm, suggesting the highly ordered internal packing of CPT molecules. The negative peak around 223 nm corresponds to hydrogen bonding interactions among the peptide segments. SAPD 2 solution presents similar CD pattern to that of SAPD1, but with significantly decreased intensity, revealing that these two building units have similar interior molecular packing (FIG. 213B). The lower intensity of SAPD 2 can be probably attributed to a looser CPT packing as a result of increased hydrophilic-lipophilic ratio and steric repulsive force posed within the peptide auxiliaries that weakens the π-π stacking among CPT units. These findings are also consistent with TEM results (FIGS. 20A, 20B) that both SAPD 1 and 2 form filamentous assemblies, but the length of assembled SAPD 1 is much longer than that of SAPD 2. The CD spectra of SAPD 3 and 4 both show a small hump between 350-400 nm, a blue-shifted bisignate signal at 256 nm and a negative peak around 204 nm, which are drastically different from those of SAPDs 1 and 2 (FIG. 21B). The lack of typical hydrogen bonding absorption indicates that SAPD 3 and 4 may not be able to form any stable SPs at this studied concentration (200 μM), and the chromophore absorptions could be possibly attributed to intramolecular CPT association within an individual prodrug, along with some loose intermolecular associations. These observations suggest that the number of OEG repeat units is critical for the formation of stable SPs, and the increased OEG chains would raise the concentration threshold for directional supramolecular growth of SAPD monomers.

### Example 11

Stability of SAPDs in Protein Environments. The interactions of nanoparticles with serum proteins are known to impact the disassociation of supramolecular assemblies in biologically relevant environment. To assess the stability of assembled SAPDs in more complex biological media, we performed CD experiments to investigate their long-term stability in various serum environments. SAPDs solutions (2 mM) were diluted using rat plasma (FIG. 21C), fetal bovine serum (FBS), and mice plasma to yield a final concentration of 200 μM, which were then aged overnight before CD measurement was taken. We found no noticeable changes in the absorptions of SAPD 1 and 2 in the protein environments (FIG. 21C) compared with those in aqueous solution (FIG. 21B), while some slight changes can be observed for SAPD 3 and 4. These results show that serum proteins had limited impact on the stability of SAPD 1 and 2 assemblies, but could be a factor destabilizing the assemblies of SAPDs 3 and 4. We also performed experiments to assess their kinetic stability upon plasma dilution, mimicking the dissociation process of SPs after intravenous injection. Solutions of SAPD 1 and 2 (2 mM) were diluted to 100 μM, 50 μM, 25 μM, 10 μM, and 5 μM in rat plasma, and time-dependent CD spectra were recorded at 5 min, 1 h, 4 h, and 12 h. By monitoring the absorption intensity at 389 nm (FIG. 21D), we found that SAPD 1 is highly stable upon dilution over time at concentrations above 25 μM. However, at 10 μM and 5 μM, the absorption at 5 min was observed to drop by 10% and 15%, respectively. At 12 h, the decrease reached 22% and 33%, respectively. SAPD 2 demonstrated a similar trend, but showing a higher propensity to dissociate as a result of its higher CMC value. These results suggest that the SAPD assemblies tend to disassociate upon dilution and the dissociation became apparent when the concentration of the diluted solutions drops near their CMCs.

### Example 12

In Vitro Drug Release Assessment of SAPDs in Physiological Environments. We next discovered that the four SPs possess very different in vitro drug release profiles in both PBS buffer and rat plasma. In these experiments, we first prepared a series of sample solutions to investigate the drug release of four SAPDs at the concentration of 200 μM in PBS buffer at 37° C. with or without 10 mM GSH, respectively (FIGS. 21E and 21F). FIG. 21E shows the summary of drug release of SAPDs over 60 minutes in the presence of GSH. Clearly, the drug release rate is SAPD1<SAPD 2<SAPDs 3 and 4, with 8% of SAPD1, 45% of SAPD2, 93% of SAPD3, and 92% of SAPD4 degraded within 5 min (FIG. 21E) evidently demonstrating that more stable SPs show increased resistance to GSH-relevant cleavage compared with the less stable ones. It is highly plausible that SAPDs in the assembled state could shield the hydrophobic CPTs and biodegradable linkers from the external environment and thus hinder the liberation of drugs. At this studied concentration, more SAPD1 exists in the SP form than SAPD2, while SAPD 3 and 4 mostly exist in the monomeric forms thus showing little resistance to GSH. FIG. 21F presents the chemical degradation profile of SAPDs over 120 h in the absence of GSH. In the absence of GSH, hydrolysis of the carbonate ester linker is considered mainly responsible for the prodrug degradation. Similar to the findings in the GSH environment, SAPD1 exhibits the most resistance toward hydrolytic cleavage with 96% of conjugates remaining intact, compared to 87% of SAPD2 and less than 60% of SAPD 3 and 4 after 120 h incubation (FIG. 21F). We also repeated this drug release experiment in rat plasma containing a myriad of proteins and enzymes (FIGS. 21G and 21H). The same drug release trend of SAPD1<SAPD 2<SAPDs 3 and 4 was observed, albert with slightly faster drug release rates relative to those in PBS, likely caused by protein-promoted dissociation and enzyme-induced degradation. The percentages of SAPDs 1-4 of remaining drugs in plasma at 96 h without GSH are 89%, 78%, 15%, and 18% (FIG. 21H), respectively, in comparison to 95%, 86%, 63% and 61% in PBS (FIG. 21F). Altogether, these results clearly suggest that SPs with a lower CMC value are more resistant to disassemble and liberate the free drugs in physiological environments.

### Example 13

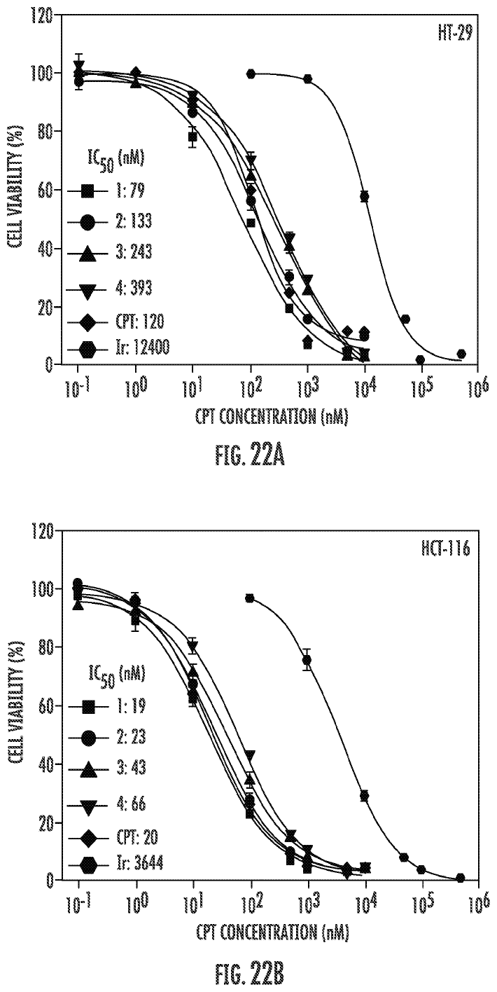

Dose-Response Inhibition against Colon Cancer Cells. Because of the effective release of potent CPT in a GSH rich environment, all SAPDs were found to demonstrate great efficacy against cancer cells. The in vitro cytotoxicities of SAPDs were evaluated against HT-29 and HCT-116 human colorectal cancer cells through a dose-response relationship assay based on CPT concentration using the SRB method. SAPDs of various concentrations were incubated with cancer cells for 72 h with both free CPT and irinotecan, a clinically used prodrug of CPT for treatment of colorectal cancer, as controls (FIG. 22). All the prodrugs exhibited a higher potency against cancer cells compared with irinotecan, which must be hydrolyzed prior to exerting its therapeutic efficacy. Although showing slight variations, the IC₅₀ values of SAPDs are within similar ranges, which are two orders of magnitude lower than that of irinotecan in both cell lines. These results led us to conclude that the design of GSH-responsive etcSS linker enables SAPDs to undergo a rapid GSH-induced restoration of potent free CPT that is much faster than hydrolysis of irinotecan, and the differences in CMC values and the related free drug release rate are not reflected on their in vitro efficacy against cancer cells.

### Example 14

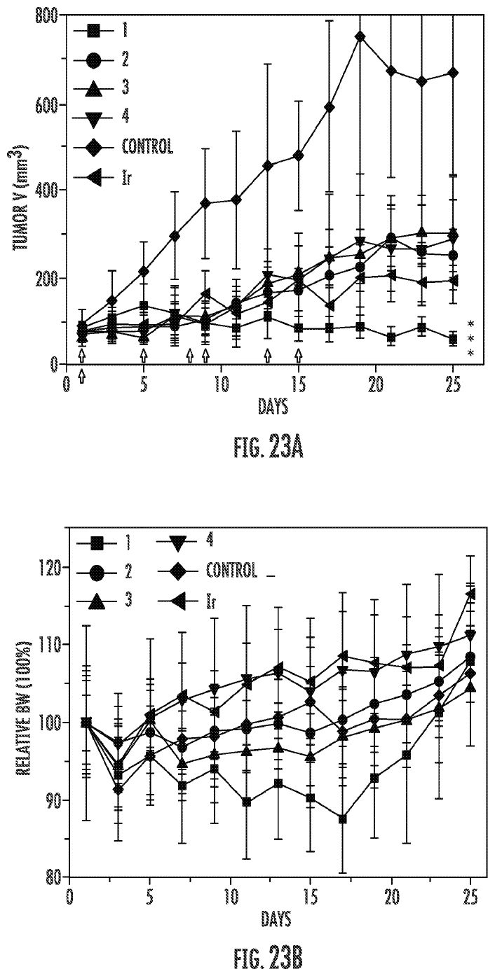

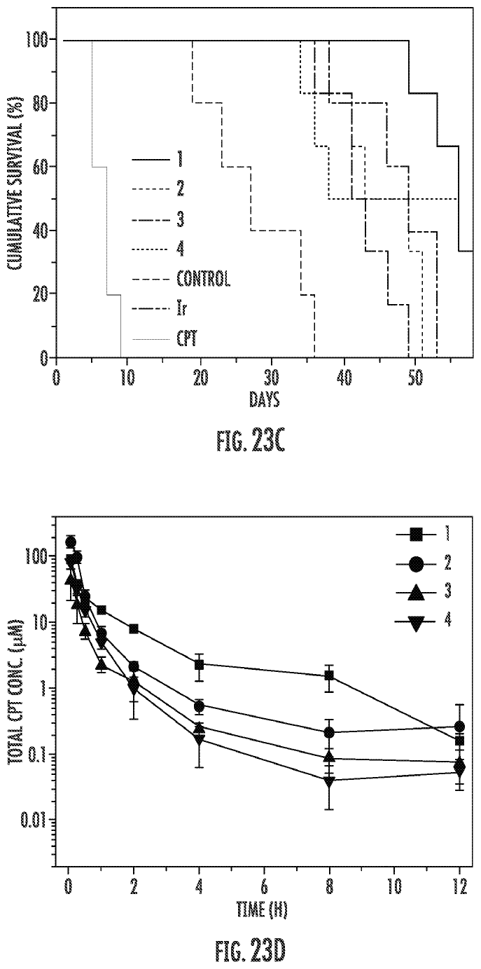

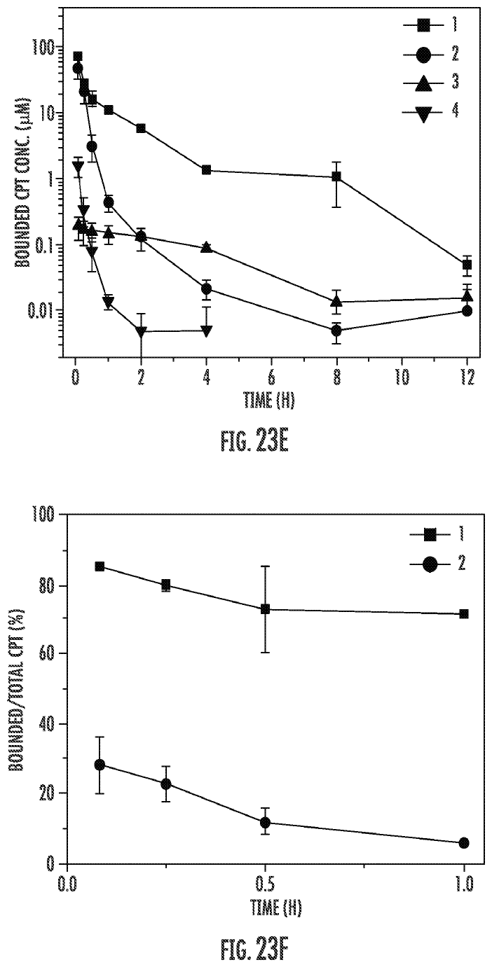

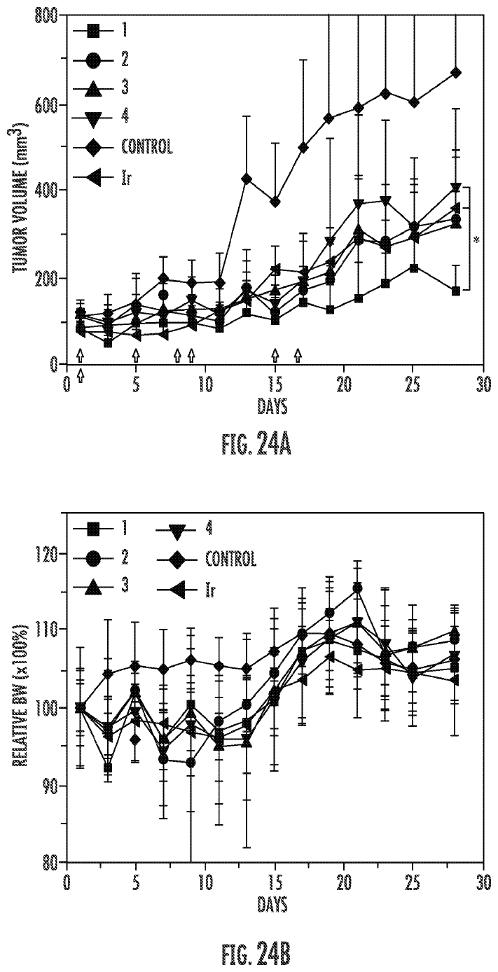

Antitumor Performance of SAPDs of Various CMCs at the Same Dose. To better understand the role of CMC in its in vivo performance, we next assessed antitumor effect of SAPDs of various CMCs in a HT-29 mouse xenograft model through intravenous (i.v.) administration of four prodrugs at a dose of 10 mg/kg (CPT equivalent) on days 1, 5, 9 and 13 (n=6 for each group) with PBS (n=5) and free CPT (9 mg/kg, n=5) as controls. Hydrophobic free CPT was dissolved in a mixture of DMSO/ethanol/PEG-400/water at a volumetric ratio of 1:1:2:1, and administrated through intraperitoneal (i.p.) injection to minimize the toxicity of organic solvents (60). Although i.p. injection of organic solvent is tolerable, administration of free CPT at 9 mg/kg still resulted in the death of all five mice after the second dose due to the associated severe toxicity of the free CPT drug (FIG. 23C). In addition, irinotecan (n=5) at its reported MTD of 100 mg/kg was also intraperitoneally injected on days 1, 8 and 15 as another control. Significant tumor regression (FIG. 23A) with much improved survival (FIG. 23C) was observed in mice treated with all SAPDs compared with the control group. SAPD 1 showed the best tumor suppression activity with a mean tumor volume of 60 mm³ on day 25 compared with 255 mm³, 304 mm³, 286 mm³ and 194 mm³ for SAPD 2-4 and irinotecan, respectively (FIG. 5A). In addition, the administration of SAPD 1 increased the median survival from 27 days (the control group) to 56 days, while the median survivals of other groups were 46 days (SAPD 2), 42 days (SAPD 3), 47 days (SAPD 4) and 51 days (irinotecan), respectively. These results suggest that the most stable SPs assembled from SAPD 1 exhibited the best efficacy compared with other SAPDs when administrated at the same dose. One possible explanation could be that less stable SPs may dissociate into more monomeric forms of the prodrugs upon plasma dilution, which is subject to a rapid renal clearance. Importantly, one could notice that SAPD 1 generated the most toxicity according to the body weight change, although it is within acceptable toxicity range (FIG. 23B).

### Example 15

In Vivo Circulation Study of SAPDs. To provide more insight into the circulation fate of SAPD assemblies in vivo, we collected the pharmacokinetic profiles of the four SAPD Sps in Sprague Dawley (SD) rats (n=3 for each prodrug) through i.v. injection at the dose of 10 mg/kg (CPT equivalent). The initial concentration of SAPDs upon plasma dilution is roughly estimated to be around 150 μM (data not shown), a value above the CMCs of SAPD 1 and 2 but below those of SAPDs 3 and 4. We found that the SAPD 1 group showed the slowest clearance of drugs from the plasma, followed by SAPD 2, SAPD 3 and SAPD 4. As shown in FIG. 23D, the total CPT concentrations for SAPDS 1-4 are 15.3 μM, 7.0 μM, 2.8 μM, and 4.7 μM at 1 h, and 8.1 μM, 2.2 μM, 1.3 μM, 1.0 μM at 2 h, respectively. More importantly, SAPD 1 maintained the lowest degradation in plasma (FIG. 23E), with 86% of CPT remained in the bounded form at 5 min in comparison to only 28% for SAPD 2 (FIG. 5F). Even at 1 h, more than 72% of total CPT retained the conjugate form (FIGS. 23E and 23F). In sharp contrast, both SAPD 3 and SAPD 4 rapidly broke down into free CPT upon injection, with more than 98% CPT released within 5 min (FIGS. 23D and 23F), suggesting that after i.v. injection their assemblies quickly dissociate into monomeric form upon plasma dilution and that their unassembled forms are vulnerable to in vivo enzymatic/hydrolytic degradation. These results are consistent with our in vitro studies, further supporting the notion that CMCs represents an important character to determine the morphological and structural integrity of supramolecular assemblies during circulation.

### Example 16

Systemic Toxicity and MTD Determination of SAPDs. Given that the therapeutic index of a drug is determined by both systemic toxicity and therapeutic efficacy, it is important to investigate the role of CMC of a SP in its systemic toxicity. We then studied the MTD of SAPDs by a dose escalation study in healthy female athymic nude mice (Table 1 and 2), which is defined by the largest dose given to a rodent that did not result in more than a 20% body weight loss or death. We previously found that SAPD 1-4 have MTDs of 24, 54, 72 and 72 mg/kg, respectively. The MTD trend of SAPD1<SAPD 2<SAPD 3 and SAPD 4 (Table 1 and 2), along with body weight fluctuation in the above-mentioned efficacy study (FIG. 23B), led us to draw the conclusion that the lower the CMC of a SP, the lower its MTD and the higher the drug's systemic toxicity. The decrease of MTD upon using stable nanostructures as drug carriers was also reported in the design of liposomal irinotecan. Previous studies have shown that encapsulation of irinotecan into liposomes showed higher toxicity than the free irinotecan in tumor-free SCID/Rag-2M mice and administration of irinotecan-encapsulated liposome at the MTD of free irinotecan resulted in significant body weight loss of studied mice. In addition, the MTD of ONIVYDE® monotherapy at 3-week interval was reported as 120 mg/m² in clinic compared with that of 320 mg/m² for irinotecan. The corroboration of our findings with these report suggest that it is likely that small molecule drugs can undergo a rapid clearance from the body that largely reduces their bioavailability. The liposomal formulation protects the drugs within stable nanostructures, which could improve the circulation, but at the same time increase the drugs' accumulation in the healthy organs. Therefore, we speculate that SAPD assemblies in more stable forms enable a longer retention time by reducing its body clearance, which could result in higher uptake by the major organs and thus the higher toxicity at the same dose level.

TABLE 1
Summary of maximum tolerated dose (MTD) study of SAPD 1

| Prodrug | Dose (mg/kg) | Maximum % BW loss (day) | Survival/Total |
|---|---|---|---|
| SAPD1 | 54 | 20 (3) | 0/3 |
| | 36 | 21.2 (3) | 0/3 |
| | 30 | 20.6 (4) | 1/3 |
| | 24 | 9.8 (3) | 3/3 |
| | 18 | 6.5 (1) | 3/3 |
| | 15 | 5.0 (2) | 3/3 |
| | 9 | 5.1 (1) | 3/3 |
| | 4.5 | 0 | 3/3 |

TABLE 2
Maximum tolerated dose (MTD) of SAPD 2 - 4 by dose
escalation studies in healthy athymic nude mice.

| Prodrug | Dose (mg/kg) | Maximum % BW loss (day) | Survival/Total |
|---|---|---|---|
| SAPD 2 | 108 | n.d. | 0/3 |
| | 72 | 11.4 (2) | 2/3 |
| | 54 | 3.5 (2) | 3/3 |
| | 45 | 4.0 (2) | 3/3 |
| | 36 | 2.1 (6) | 3/3 |
| | 18 | 6.2 (5) | 3/3 |
| SAPD 3 | 108 | n.d. | 2/3 |
| | 72 | 0 | 3/3 |
| | 54 | 1.6 (1) | 3/3 |
| | 45 | 2.5 (4) | 3/3 |
| | 36 | 1.4 (4) | 3/3 |
| | 18 | 5.5 (4) | 3/3 |
| SAPD 4 | 108 | n.d. | 1/3 |
| | 72 | 1.2 (1) | 2/3 |
| | 54 | 3.9 (3) | 3/3 |
| | 45 | 0 | 3/3 |
| | 36 | 7.2 (2) | 3/3 |
| | 18 | 3.1 (5) | 3/3 |

A single i.v. injection of SAPDs was administrated, and body weights of mice were recorded for two weeks (n=3 for each group). All the doses are CPT equivalent. SAPD 1-4 have MTDs of 24 mg/kg, 54 mg/kg, 72 mg/kg, and 72 mg/kg, respectively. If the body weight of a mouse decreases more than 20%, the mouse will be euthanized and counted as a death.

### Example 17

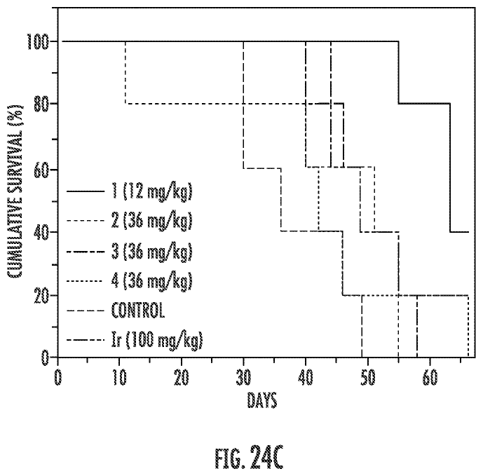

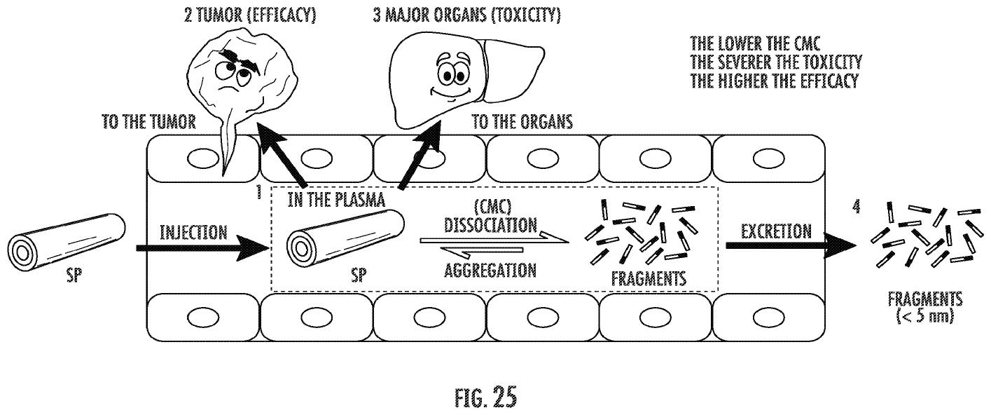

Antitumor Performance of SAPDs at Their Respective MTDs. Given that SAPDs 1-4 showed excellent tolerability in the above-mentioned efficacy study (FIG. 23) and that intensification of dosage to improve treatment outcome is often favored within a drug's tolerability, we decided to elevate the dose to assess if a better tumor inhibition efficacy can be achieved. Based on our previous experiences, the MTD of multiple injections (three doses and four days a dose) could be around ½ of the MTD of a single injection (Tables 1 and 2). For example, the MTD of SAPD 1 of multiple injections is 12 mg/kg that is ½ of the MTD (24 mg/kg) of a single injection. Thus, in the following efficacy study, we used the dose of ½ MTD for each prodrug that is 12 mg/kg (½ MTD) for SAPD 1, and 36 mg/kg for both SAPD 3 and 4, respectively. Regardless of a slightly lower MTD (54 mg/kg) of SAPD 2 compared with that of SAPD 3 and 4 (72 mg/kg), we decided to use 36 mg/kg for consistency; the irinotecan group and PBS group were also used as controls (FIG. 24). Again, all the SAPDs significantly suppressed the tumor growth at the dose of their estimated MTDs relative to the control group (FIG. 24A). The treated mice showed a mean tumor volume of 172 mm³, 330 mm³, 322 mm³, 408 mm³ and 358 mm³ for SAPD 1-4 and irinotecan, respectively, on day 28 (FIG. 24A). SAPD 1 was a bit more effective than other SAPDs, however it is not statistically significant as analyzed by one-way ANOVA (p>0.05), except relative to SAPD 4 (p=0.02). Furthermore, SAPD 2 showed systemic toxicity with one treatement related death (FIG. 24C), which also indicates that the predetermined dose could be higher than the MTD of SAPD 2. A similar trend was observed in survival that SAPD 1 slightly improved the survival of mice compared with the other groups. Although we cannot rule out that further increase of the dose of SAPD 2-4 would lead to an even better efficacy (it may also lead to more severe toxicity), our current results suggest that the efficacy of SAPD 2-4 does not exceed SAPD 1 even at their respective estimated MTDs.

These in vivo experimental results collectively demonstrate the significant role of CMC values in determining the circulation, therapeutic efficacies and systemic toxicities of supramolecular polymers. FIG. 25 illustrates a scheme of the possible four destinations that therapeutic supramolecular polymers could reach after systemic administration, which are closely related to their circulation, efficacy, and toxicity. Following intravenous injection, supramolecular polymers could be contained within plasma (circulation), accumulate in tumorous tissues (efficacy) or healthy organs (toxicity), or get cleared out through the excretion systems. As a result of their supramolecular nature, all SAPD SPs are expected to undergo spontaneous dissociation after plasma dilution into fragmented pieces and monomeric units. From the perspective of pharmacokinetics, smaller fragments (<6 nm) and monomeric prodrugs can be rapidly excreted through the renal system (FIG. 25). This explains why SAPDs 2-4 had a higher respective MTD than SAPD 1 because more of their monomers were likely excreted out of the studied mice. Since less SAPDs 2-4 are left within the body, it consequently lowered the accumulation in both tumor and healthy tissues thus leading to reduced treatment efficacy and increased MTD. In contrast, a larger percentage of SAPD 1 is expected to assume the supramolecular form during the circulation that are too big for renal clearance, so as to improve accumulation in tumors for better treatment efficacy. Although SAPD 2 has a CMC value (10.1 μM) close to that of SAPD 1 (2.7 μM) and behaved similarly in their in vitro stability under the quiescent conditions (FIGS. 3C, and. 3D) and prodrug release in the absence of GSH (FIG. 3F and FIG. 3H), SAPD 2 filaments were observed to rapidly dissociate into monomeric units after intravenous administration (FIG. 5E). As a result, SAPD 2 demonstrated a much higher MTD (54 vs. 24 mg/kg) and a much reduced efficacy in tumor suppression. These observations suggest that the CMCs, in vitro stability and drug release data measured under quiescent conditions only provide qualitative information to predict the drug's in vivo performance. It is the drug's in vivo stability and pharmacokinetic profile that afford more reliable prediction of its in vivo efficacy.

On basis of our in vivo study results, SAPD 1 appears to be the best candidate for further development as it revealed the best efficacy in suppressing tumor growth at the same dosage (10 mg/kg), and also a comparable efficacy even when SAPDs 2-4 were administered at their respective MTD. However, it should be noted that although SAPD 1 demonstrated the best in vivo efficacy, it also revealed the greatest toxicity by having the lowest MTD. Thus, an optimal CMC value should exist to balance the healthy organ toxicity with the tumor treatment efficacy. This statement also eludes that permanent locking of supramolecular nanostructures through shell or internal crosslinking may not represent the best strategy as it would also boost toxicity to healthy organs, and also highlights the important role that supramolecular assemblies could play in the development of more effective drug carriers. In drug development, therapeutic index is an important measure of therapeutic efficacy relative to the toxicity it may cause. A higher therapeutic index is often preferred as it suggests a favorable safety and efficacy profile. In the present case, although we cannot directly assess the therapeutic index for each SAPD design, we can envision an improved therapeutic index for all the studied SAPDs over the parent drug CPT. The present studies also reveal the tunability of therapeutic index through molecular engineering of self-assembling prodrugs given their difference in MTD and treatment efficacy.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. T. Aida, E. W. Meijer, S. I. Stupp, Functional supramolecular polymers. Science 335, 813-817 (2012).
2. L. Brunsveld, B. J. B. Folmer, E. W. Meijer, R. P. Sijbesma, Supramolecular polymers. Chem. Rev. 101, 4071-4097 (2001).
3. W. Zhang, W. S. Jin, T. Fukushima, A. Saeki, T. Aida, Supramolecular linear heterojunction composed of graphite-like semiconducting nanotubular segments. Science 334, 340-343 (2011).
4. A. Schenning, E. W. Meijer, Supramolecular electronics; nanowires from self-assembled pi-conjugated systems. Chem. Commun. 26, 3245-3258 (2005).
5. Y. N. Hong, J. W. Y. Lam, B. Z. Tang, Aggregation-induced emission. Chem. Soc. Rev. 40, 5361-5388 (2011).
6. Y. Gao, J. F. Shi, D. Yuan, B. Xu, Imaging enzyme-triggered self-assembly of small molecules inside live cells. Nat. Commun. 3, 1033 (2012).
7. J. D. Hartgerink, E. Beniash, S. I. Stupp, Self-assembly and mineralization of peptide-amphiphile nanofibers. Science 294, 1684-1688 (2001).
8. Y. Kuang, B. Xu, Disruption of the dynamics of microtubules and selective inhibition of glioblastoma cells by nanofibers of small hydrophobic molecules. Angew. Chem. Int. Ed 52, 6944-6948 (2013).
9. G. A. Silva, C. Czeisler, K. L. Niece, E. Beniash, D. A. Harrington, J. A. Kessler, S. I. Stupp, Selective differentiation of neural progenitor cells by high-epitope density nanofibers. Science 303, 1352-1355 (2004).
10. J. P. Hill, W. S. Jin, A. Kosaka, T. Fukushima, H. Ichihara, T. Shimomura, K. Ito, T. Hashizume, N. Ishii, T. Aida, Self-assembled hexa-peri-hexabenzocoronene graphitic nanotube. Science 304, 1481-1483 (2004).
11. A. G. Cheetham, P. Zhang, Y. A. Lin, L. L. Lock, H. Cui, Supramolecular nanostructures formed by anticancer drug assembly. I Am. Chem. Soc. 135, 2907-2910 (2013).
12. M. J. Webber, E. A. Appel, E. W. Meijer, R. Langer, Supramolecular biomaterials. Nat. Mater. 15, 13-26 (2016).
13. J. A. MacKay, M. N. Chen, J. R. McDaniel, W. G. Liu, A. J. Simnick, A. Chilkoti, Self-assembling chimeric polypeptide-doxorubicin conjugate nanoparticles that abolish tumours after a single injection. Nat. Mater. 8, 993-999 (2009).
14. Y. Bae, N. Nishiyama, S. Fukushima, H. Koyama, M. Yasuhiro, K. Kataoka, Preparation and biological characterization of polymeric micelle drug carriers with intracellular pH-triggered drug release property: Tumor permeability, controlled subcellular drug distribution, and enhanced in vivo antitumor efficacy. Bioconjugate Chem. 16, 122-130 (2005).
15. Y. Pommier, Topoisomerase I inhibitors: camptothecins and beyond. Nat. Rev. Cancer 6, 789 (2006).
16. H. Fenniri, M. Packiarajan, K. L. Vidale, D. M. Sherman, K. Hallenga, K. V. Wood, J. G. Stowell, Helical rosette nanotubes: Design, self-assembly, and characterization. J. Am. Chem. Soc. 123, 3854-3855 (2001).
17. Z. Huang, S. K. Kang, M. Banno, T. Yamaguchi, D. Lee, C. Seok, E. Yashima, M. Lee, Pulsating tubules from noncovalent macrocycles. Science 337, 1521-1526 (2012).
18. B. M. W. Langeveld-Voss, D. Beljonne, Z. Shuai, R. A. J. Janssen, S. C. J. Meskers, E. W. Meijer, J. L. Bredas, Investigation of exciton coupling in oligothiophenes by circular dichroism spectroscopy. Adv. Mater. 10, 1343-1348 (1998).
19. P. Greenspan, E. P. Mayer, S. D. Fowler, Nile Red—a selective fluorescent stain for intracellular lipid droplets. J. Cell. Biol. 100, 965-973 (1985).
20. S. Liu, P. Zhang, S. R. Banerjee, J. D. Xu, M. G. Pomper, H. Cui, Design and assembly of supramolecular dual-modality nanoprobes. Nanoscale 7, 9462-9466 (2015).
21. Y. Geng, P. Dalhaimer, S. S. Cai, R. Tsai, M. Tewari, T. Minko, D. E. Discher, Shape effects of filaments versus spherical particles in flow and drug delivery. Nat. Nanotechnol. 2, 249-255 (2007).
22. J. Israelachvili, Intermolecular and surface forces. (Academic Press, ed. 3rd, 2011).
23. R. H. J. Mathijssen, R. J. van Alphen, J. Verweij, W. J. Loos, K. Nooter, G. Stoter, A. Sparreboom, Clinical pharmacokinetics and metabolism of irinotecan (CPT-11). Clin. Cancer Res. 7, 2182-2194 (2001).
24. T. Schluep, J. J. Cheng, K. T. Khin, M. E. Davis, Pharmacokinetics and biodistribution of the camptothecin-polymer conjugate IT-101 in rats and tumor-bearing mice. Cancer Chemother. Pharmacol. 57, 654-662 (2006).

1. A self assembling prodrug comprising one or more hydrophobic drug molecules covalently linked to at least one or more biodegradable carbonate linkers which are covalently linked to one or more hydrophilic peptides.

2. The prodrug composition of claim 1 comprising the following formula: 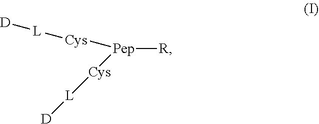
wherein D is a hydrophobic drug molecule, L is a hydrolysable linker, Cys is cysteine, Pep is a hydrophilic peptide of at least two amino acids with a free side chain, and R is H, or a hydrophilic molecule of choice.

3. The prodrug composition of claim 1, wherein the hydrophobic drug molecules comprise camptothecin, and variants thereof.

4. The prodrug composition of any of claims 1 to 3, wherein the one or more biodegradable carbonate linkers comprise disulfanylbutanoate (buSS) and disulfanylethanoate (etcSS).

5. The prodrug composition of any claims 1 to 4, wherein the one or more hydrophilic peptides can comprise hydrophilic polymers.

6. The prodrug composition of any claims 1 to 4, wherein the one or more hydrophilic peptides can be cationic, anionic, zwitterionic peptides.

7. The prodrug composition of any claims 1 to 4, wherein the one or more hydrophilic peptides can comprise a chelating moiety.

8. A prodrug tubustecan compound having the following formula:

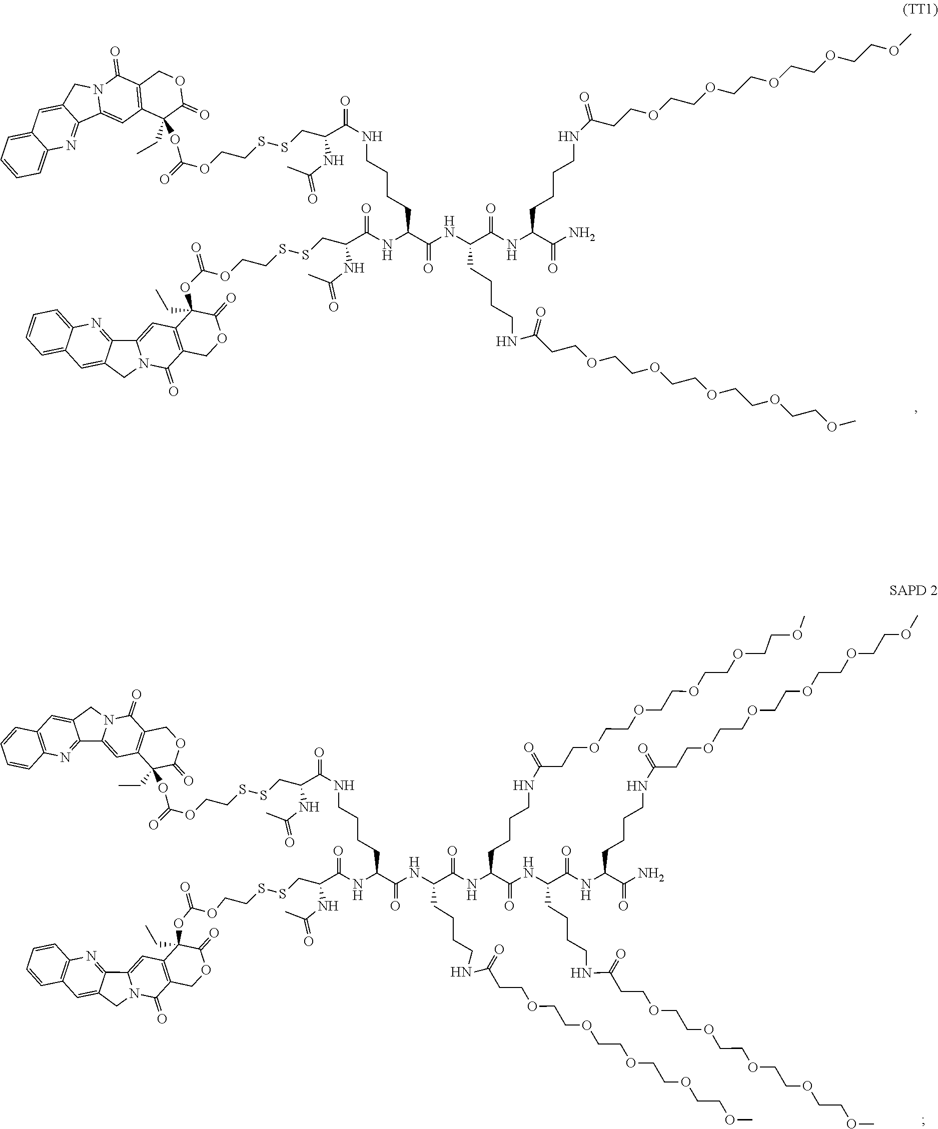

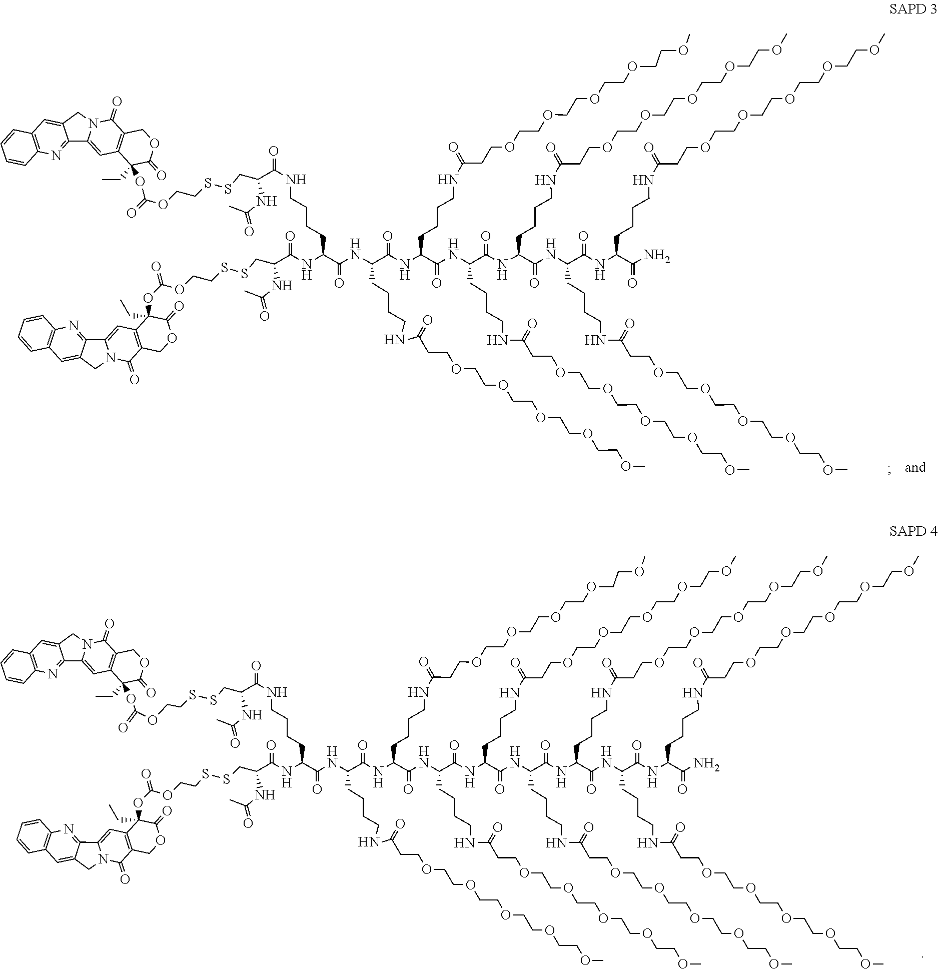

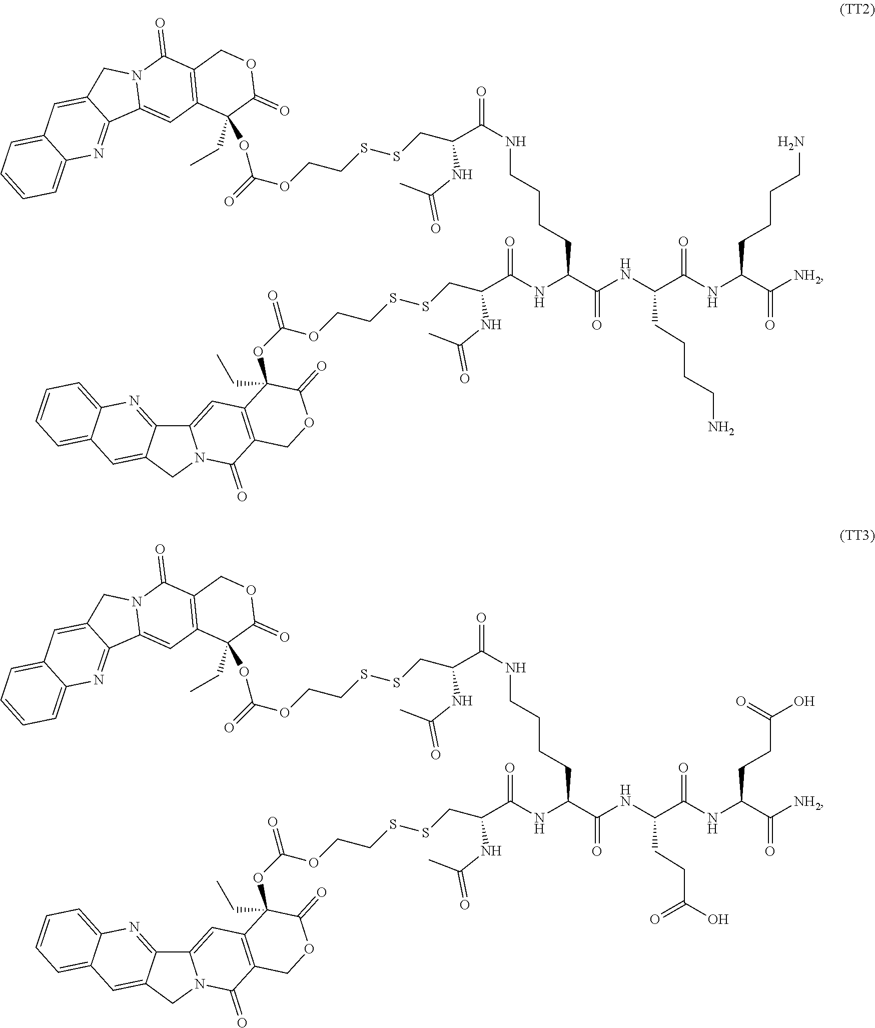 [SMILES: CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCCCN)C(=O)N[C@@H](CCCCN)C(N)=O)C(=O)OCc2c1cc1n(c2=O)Cc2cc3ccccc3nc2-1 ; CC[C@]1(OC(=O)OCCSSC[C@@H](NC(C)=O)C(=O)N[C@@H](CCCCNC(=O)[C@@H](CSSCCOC(=O)O[C@]2(CC)C(=O)OCc3c2cc2n(c3=O)Cc3cc4ccccc4nc3-2)NC(C)=O)C(=O)N[C@@H](CCC(=O)O)C(=O)N[C@@H](CCC(=O)O)C(N)=O)C(=O)OCc2c1cc1n(c2=O)Cc2cc3ccccc3nc2-1]

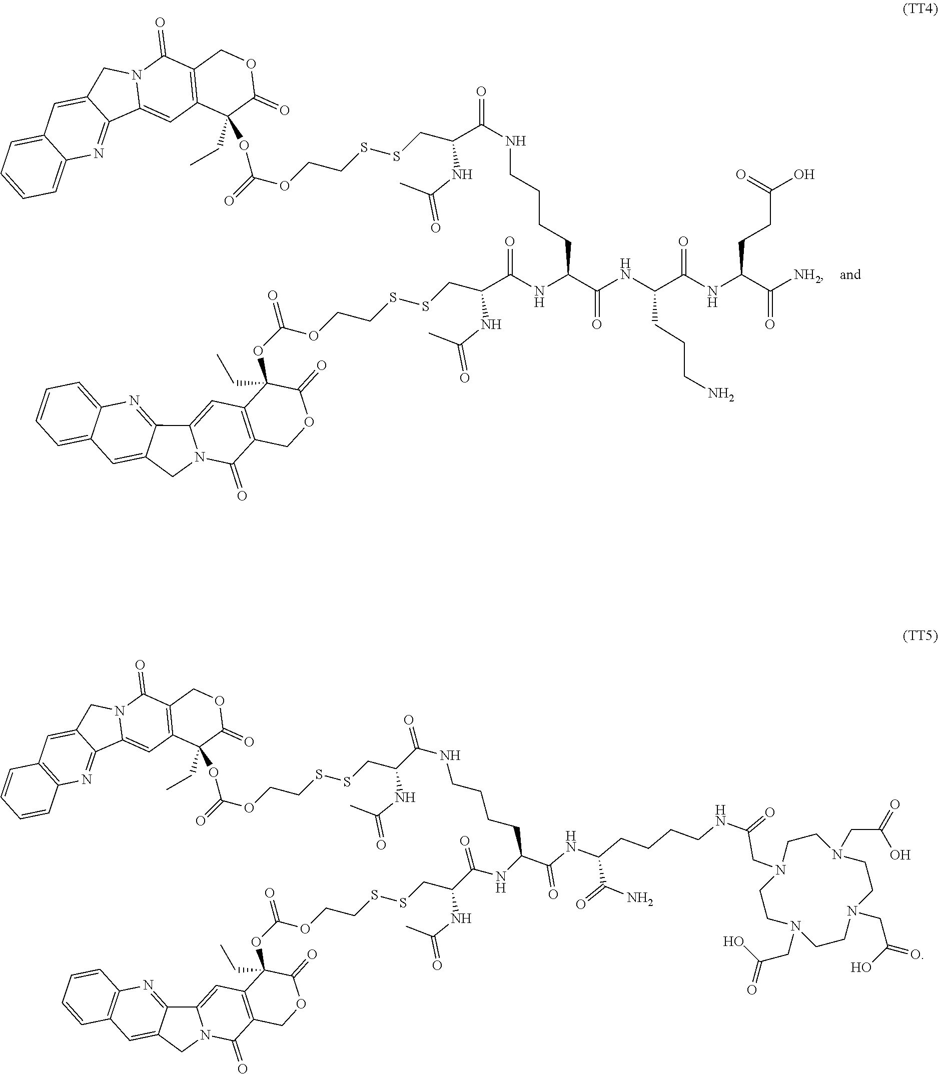

9. A prodrug composition comprising the compounds of any of claims 1 to 8, and a pharmaceutically acceptable carrier.

10. The prodrug composition of claim 9, further comprising at least one additional biologically active agent.

11. The prodrug composition of either of claim 9 or 10, further comprising at least one detectable moiety.

12. A method for treating cancer in a subject comprising administering to the subject an effective amount of at least one or more prodrug compounds of claim 8 or the compositions of any of claims 1 to 7 and 9 to 11.
